(19) United States
(12) Patent Application Publication
(10) Pub. No.: US 2026/0193165 A1
(43) Pub. Date: Jul. 9, 2026

(54) Polyol-Derived Compounds

(71) Applicant: ARXADA AG, Visp (CH)

(72) Inventors: Ulrich Mayerhoeffer, Oberdorf (DE); Sebastian Benz, Visp (CH); Arne Jan Stepen, Visp (CH); Martin Lagger, Visp (CH); Selina Beutter, Visp (CH)

(21) Appl. No.: 19/121,639

(22) PCT Filed: Oct. 18, 2023
(86) PCT No.: PCT/EP2023/078941

§ 371 Date: Apr. 16, 2025

(30) Foreign Application Priority Data
Oct. 18, 2022 (EP) 22202302.0

(51) Int. Cl. C07C 69/675

(52) U.S. Cl. CPC C07C 69/675

(57) ABSTRACT

The present invention relates to polyol-derived compounds and processes preparing the same.

### TECHNICAL FIELD

The present invention relates to polyol-derived compounds and processes preparing the same.

### TECHNOLOGICAL BACKGROUND

Acetoacetylated polyalcohols and β-hydroxy butyric acid esters of polyalcohols prepared therefrom are valuable compounds with a versatile utilization for example as parenteral nutrients or for the treatment of certain diseases.

US 2019/117612 A1 pertains to the field of migraine headaches and the management of the symptomology thereof using 3-hydroxybutyrate glycerides.

US 2018/193300 A1 pertains to a method of treatment of mild to moderate non-penetrating closed traumatic brain injury and mild to moderate traumatic brain injury due to surgical intervention using 3-hydroxybutyate glycerides.

Hence, ketone bodies like acetoacetate (AA) and β-hydroxybutyrate (BHB) are believed to have a positive impact on brain health and may alleviate brain related disease and the associated symptoms. Therefore it can be beneficial to supplement either AA or BHB to improve health of a subject such as brain health.

In the human body, AA and BHB are in a natural equilibrium wherein the interconversion of AA to BHB and BHB to AA is catalysed by the enzyme β-hydroxybutyrate dehydrogenase involving nicotinamide adenine dinucleotide (NAD) (cf. for example H. Kolb et al. “Ketone bodies: from enemy to friend and guardian angel”, BMC Med., 2021, 19 (1), 313). The common physiological AA:BHB ratio in the human body ranges from 1:3 to 1:2 (AA:BHB).

In view of the natural equilibrium between AA and BHB in the human body, it would be desirable to supplement AA and BHB at the same time, preferably in the same ratio as present in the human body in order to maintain and respect the natural physiological equilibrium between BHB and AA when supplementing ketone bodies.

So far, molecules that contain either BHB or AA were used to increase ketone body levels in the body. However, even if mixtures of compounds containing either AA or BHB are used for ketone body supplementation, such mixtures of compounds suffer from the drawback in that these compounds have different pharmacokinetics and result in uneven delivery. Therefore, in order to enhance simultaneous uptake of BHB and AA, they are advantageously incorporated into the same molecule.

Compounds to be used for supplementing AA and BHB should preferably comprise both entities in a predefined ratio that resembles the physiologic ratio in the human body.

Hence, there is a need for providing polyalcohol esters comprising both BHB and AA units in one molecule, preferably in an adjustable ratio.

There is further a need for excellent processes for the synthesis of such polyalcohol esters which comprise both BHB and AA in one molecule.

### SUMMARY OF THE INVENTION

The above needs are met by the compounds and processes of the present invention.

Accordingly, the present invention provides a compound of formula 1

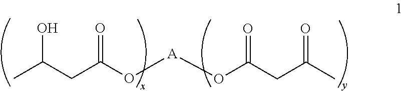

wherein
A is derived from an organic polyol with at least 3 hydroxyl groups,
x is at least 1,
y is at least 1, and
x+y is from 3 to the number of hydroxyl groups of the initial organic polyol A, and wherein the ratio of x to y is not 1:1 (x≠y).

In another aspect, the present invention provides a process for the preparation of a compound of formula 1

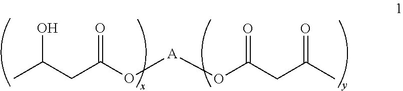

wherein
A is derived from an organic polyol with at least 3 hydroxyl groups,
x is at least 1,
y is at least 1, and
x+y is from 3 to the number of hydroxyl groups of the initial organic polyol A, and wherein the ratio of x to y is not 1:1 (x≠y);
wherein the process comprises:
   (i) reacting an organic polyol of formula 2 with diketene 3 resulting in the formation of a compound according to formula 4;

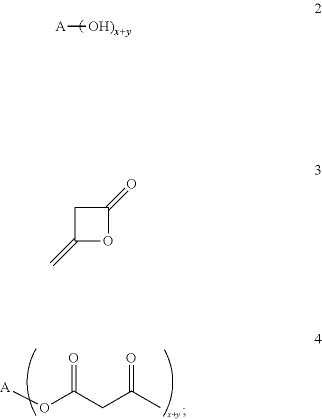

and
(ii) partially hydrogenating the compound of formula 4 by reacting the compound of formula 4 with hydrogen in the presence of a catalyst resulting in the formation of the compound according to formula 1.

In another aspect, the present invention provides a process for the preparation of a compound of formula 1

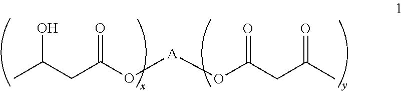

wherein
A is derived from an organic polyol with at least 3 hydroxyl groups,
x is at least 1,
y is at least 1, and
x+y is from 3 to the number of hydroxyl groups of the initial organic polyol A, and wherein the ratio of x to y is not 1:1 (x≠y);
wherein the process comprises:
   (i) partially reacting an organic polyol of formula 2 with diketene 3 resulting in the formation of a compound according to formula 5;

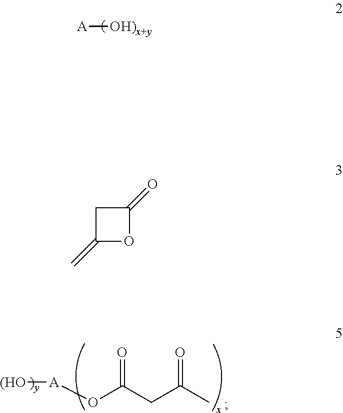

(ii) reacting the compound of formula 5 with hydrogen in the presence of a catalyst resulting in the formation of the compound according to formula 6

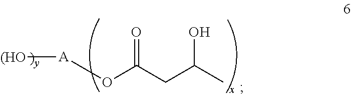

and
(iii) partially reacting the compound of formula 6 with diketene 3 resulting in the formation of the compound according to formula 1.

In another aspect, the present invention provides a compound of formula 7

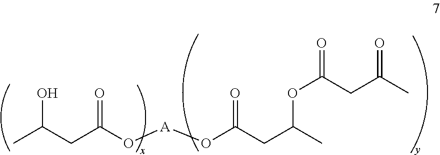

wherein
A is derived from an organic polyol with at least 3 hydroxyl groups,
x is at least 1,
y is at least 1, and
x+y is from 3 to the number of hydroxyl groups of the initial organic polyol A.

In another aspect, the present invention provides a process for the preparation of a compound of formula 7

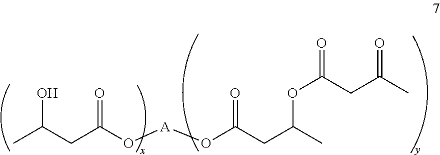

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) reacting an organic polyol of formula 2 with diketene 3 resulting in the formation of a compound according to formula 4;

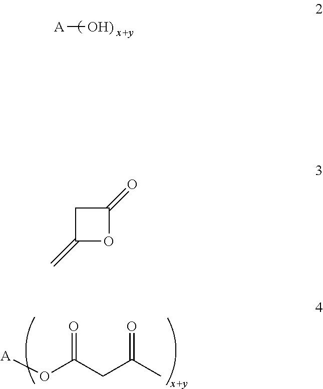

(ii) reacting the compound of formula 4 with hydrogen in the presence of a catalyst resulting in the formation of a compound according to formula 8;

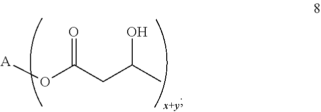

and
(iii) partially reacting the compound according to formula 8 with diketene 3 resulting in the formation of a compound according to formula 7.

In another aspect, the present invention provides a compound of formula 9

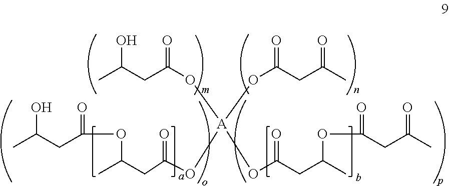

wherein
A is derived from an organic polyol with at least 3 hydroxyl groups,
m is 0, 1, 2, 3, 4, or 5,
n is 0, 1, 2, 3, 4, or 5,
o is 0, 1, 2, 3, 4, or 5,
p is 0, 1, 2, 3, 4, or 5,
at least two of m, n, o, and p are not 0,
m+n+o+p is from 2 to the number of hydroxyl groups of the initial organic polyol A,
a is 1-10, and
b is 1-10, and wherein

$$n + p \neq a + b + m + o .$$

In another aspect, the present invention provides a compound of formula 10

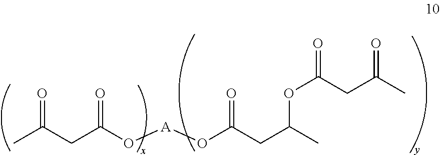

wherein
A is derived from an organic polyol with at least 3 hydroxyl groups,
x is at least 1,
y is at least 1, and
x+y is from 3 to the number of hydroxyl groups of the initial organic polyol A.

In another aspect, the present invention provides a process for the preparation of a compound of formula 10

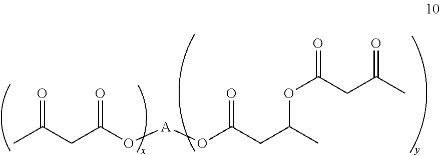

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) partially reacting an organic polyol of formula 2 with diketene 3 resulting in the formation of a compound according to formula 11;

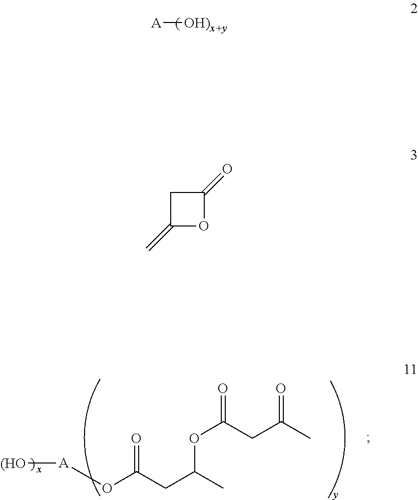

(ii) reacting the compound of formula 11 with hydrogen in the presence of a catalyst resulting in the formation of the compound according to formula 12

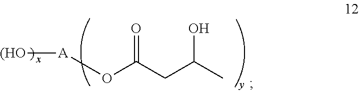

and
(iii) reacting the compound of formula 12 with diketene 3 resulting in the formation of the compound according to formula 10.

### DETAILED DESCRIPTION OF THE INVENTION

In the following, the invention will be explained in more detail.

### Definitions

In order for the present invention to be readily understood, several definitions of terms used in the course of the invention are set forth below.

According to the present invention, the term “linear or branched C₂₋₁₂ alkyl” refers to a straight-chained or branched saturated hydrocarbon group having 2 to 12 carbon atoms, such as 2, 3, 4, 5, 6, 7, 8, 9, 10, 11 or 12 carbon atoms including methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethyl propyl, 1,2,2-trimethylpropyl, 1-ethyl-1-methylpropyl and 1-ethyl-2-methylpropyl.

According to the present invention, the term “C₃₋₈ cycloalkyl” refers to a monocyclic saturated hydrocarbon group having 3 to 8 carbon ring members, such as 2, 3, 4, 5, 6, 7, or 8 carbon ring members, including cyclopropyl, cyclobutyl, cyclopentyl, cyclohexyl, cycloheptyl, and cyclooctyl.

It is to be understood that the linear or branched C₂₋₁₂ alkyl and C₃₋₈ cycloalkyl may optionally be further substituted. Exemplary substituents include hydroxy, linear or branched C₁₋₁₂ alkyl, C₃₋₈ cycloalkyl, a carboxy group, halogen, and phenyl.

According to the present invention, the term “organic polyol” refers to a linear, branched, or cyclic organic compound with 2 to 18 carbon atoms having at least three hydroxyl groups. As such, the organic polyol may have 2, 3, 4, 5, 6, 7, 8, 9, 10, 11, 12, 13, 14, 15, 16, 17, or 18 carbon atoms. In one embodiment, no more than one hydroxyl group is connected to one carbon atom. In one embodiment, the organic polyol contains only carbon, hydrogen, and oxygen atoms.

According to the present invention, the term “at least three hydroxyl groups” means that the respective compound has three or more hydroxyl groups. In one embodiment, “at least three hydroxyl groups” includes 3 to 18 hydroxyl groups such as 3, 4, 5, 6, 7, 8, 9, 10, 11, 12, 13, 14, 15, 16, 17, or 18 hydroxyl groups. In one embodiment, “at least three hydroxyl groups” includes 3 to 12 hydroxyl groups such as 3, 4, 5, 6, 7, 8, 9, 10, 11, or 12 hydroxyl groups. In one embodiment, “at least three hydroxyl groups” includes 3 to 9 hydroxyl groups such as 3, 4, 5, 6, 7, 8, or 9 hydroxyl groups. In one embodiment, “at least three hydroxyl groups” includes 3 to 6 hydroxyl groups such as 3, 4, 5, or 6 hydroxyl groups. In a preferred embodiment, “at least three hydroxyl groups” includes 3 to 5 hydroxyl groups such as 3, 4, or 5 hydroxyl groups.

It is to be understood that if not explicitly stated otherwise, all stereoisomers, conformations and configurations are encompassed by compounds and functional groups which can be present as different stereoisomers or in different conformations and configurations. For example, the term “inositol” is to be understood as to include all stereoisomers and conformations such as myo-, scyllo-, muco-, D-chiro-, neo-inositol, L-chiro-, allo-, epi-, and cis-inositol. For example, the term “hexanetriol” is to be understood as to include all hexane isomers including three hydroxyl groups such as 1,1,1-hexanetriol, 1,1,2-hexanetriol, 1,2,2-hexanetriol, 1,2,3-hexanetriol, 1,2,4-hexanetriol, 1,2,5-hexanetriol, 1,2,6-hexanetriol, 1,3,5-hexanetriol, 1,3,6-hexanetriol, 2,3,4-hexanetriol, 2,3,5-hexanetriol etc.

As used herein, the term “comprising” is to be construed as encompassing both “including” and “consisting of”, both meanings being specifically intended, and hence individually disclosed, embodiments according to the present invention.

As used herein, the articles “a” and “an” preceding an element or component are intended to be nonrestrictive regarding the number of instances (i.e. occurrences) of the element or component. Therefore, “a” or “an” is to be read to include one or at least one, and the singular word form of the element or component also includes the plural unless the number is obviously meant to be singular.

As used herein, the term “about” modifying the quantity of a substance, ingredient, component, or parameter employed refers to variation in the numerical quantity that can occur, for example, through typical measuring and handling procedures, e.g., liquid handling procedures used for making concentrates or solutions. Furthermore, variation can occur from inadvertent error in measuring procedures, differences in the manufacture, source, or purity of the ingredients employed to carry out the methods, and the like. In one embodiment, the term “about” means within 10% of the reported numerical value. In a more specific embodiment, the term “about” means within 5% of the reported numerical value.

As outlined above, subject of the present invention provides a compound of formula 1

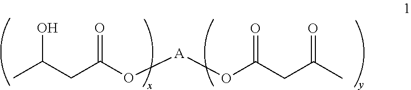

wherein
A is derived from an organic polyol with at least 3 hydroxyl groups,
x is at least 1,
y is at least 1, and
x+y is from 3 to the number of hydroxyl groups of the initial organic polyol A.

In one embodiment, the organic polyol has from 3 to 10 hydroxyl groups. Preferably, the organic polyol has from 3 to 8 hydroxyl groups, such as from 3 to 7 hydroxyl groups, from 3 to 6 hydroxyl groups, more preferably from 3 to 5 hydroxyl groups, or from 3 to 4 hydroxyl groups.

In one embodiment, x is at least 2, at least 3, or at least 4. In one embodiment, x is 1, 2, 3, 4, 5, or 6. Preferably, x is 1, 2, 3, or 4.

In one embodiment, y is at least 2, at least 3, or at least 4. In one embodiment, y is 1, 2, 3, 4, 5, or 6. Preferably, y is 1, 2, 3, or 4.

In one embodiment, x is 1 and y is 3. In one embodiment, x is 2 and y is 2. In one embodiment, x is 3 and y is 1.

In a preferred embodiment, the number of AA residues differs from the number of BHB residues (x≠y), i.e., the ratio of x to y is not 1:1. In one embodiment, the ratio of x to y is between 7:1 and 1:7, with the caveat that the ratio is not 1:1. In a further embodiment, the ratio of x to y is between 5:1 and 1:5, with the caveat that the ratio is not 1:1.

In one embodiment, x is larger than y, i.e., x>y. In a preferred embodiment, the ratio of x to y is between 7:1 and 2:1. In another preferred embodiment, the ratio of x to y is between 5:1 and 2:1. In a more preferred embodiment, the ratio is selected from the group consisting of 2:1, 3:1, 4:1, 5:1, 6:1 and 7:1, even more preferably 2:1 or 3:1.

In one embodiment, y is larger than x, i.e., x<y. In a preferred embodiment, the ratio of x to y is between 1:7 and 1:2. In another preferred embodiment, the ratio of x to y is between 1:5 and 1:2. In a more preferred embodiment, the ratio is selected from the group consisting of 1:2, 1:3, 1:4, 1:5, 1:6 or 1:7, even more preferably 1:2 or 1:3.

In one embodiment, x+y is from 3 to 10, such as from 3 to 8, from 3 to 7, from 3 to 6, from 3 to 5 or from 3 to 4. Accordingly, x+y may be 3, 4, 5, 6, 7, 8, 9, or 10, preferably 3, 4, 5, or 6, more preferably 3, 4, or 5.

In one embodiment, x+y is equal to the number of hydroxyl groups of the initial polyol A. In one embodiment, x+y is less than the number of hydroxyl groups of the initial polyol A.

In one embodiment, the organic polyol is a linear, branched, or cyclic organic compound with 2 to 18 carbon atoms having at least three hydroxyl groups. In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

In another preferred embodiment, the linear or branched C₂₋₁₂ alkyl substituted with at least 3 hydroxyl groups is selected from the group consisting of trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

Preferably, the C₃₋₈ cycloalkyl substituted with at least 3 hydroxyl groups is selected from the group consisting of cyclopentanetriol, cyclohexanetriol, cyclopentanetetrol, cyclohexanetetrol.

In one embodiment, the organic polyol is selected from the group consisting of monosaccharides, sugar alcohols, and sugar acids.

Monosaccharides generally have the chemical formula CₙH₂ₙOₙ. Monosaccharides can be classified by the number x of carbon atoms they contain (CH₂O)ₓ:trioses (x=3), tetroses (x=4), pentoses (x=5), hexoses (x=6) and heptoses (x=7).

In one embodiment, the monosaccharide is selected from tetroses, pentoses, hexoses, heptoses. Preferably, the monosaccharide is selected from aldotetroses, ketotetroses, aldopentoses, ketopentoses, aldohexosen, ketohexoses, aldoheptoses, ketoheptoses.

In one embodiment, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.

Sugar alcohols (also called polyhydric alcohols, polyalcohols, alditols or glycitols) are organic compounds, typically derived from sugars, containing one hydroxyl group (—OH) attached to each carbon atom.

In one embodiment, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.

A sugar acid is generally a monosaccharide with a carboxyl group at one end or both ends of the carbon chain. Main classes of sugar acids include aldonic acids, ulosonic acids, uronic acids, and aldaric acids. In aldonic acids, the aldehyde group (—CHO) located at the initial end (position 1) of an aldose is oxidized. In ulosonic acids, the —CH₂ (OH) group at the initial end of a 2-ketose is oxidized yielding an α-ketoacid. In uronic acids, the —CH₂ (OH) group at the terminal end of an aldose or ketose is oxidized. In aldaric acids, both ends (—CHO and —CH₂(OH)) of an aldose are oxidized.

In one embodiment, the sugar acid is selected from aldonic acids, ulosonic acids, uronic acids, aldaric acids. Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.

In one embodiment, the organic polyol is selected from the group consisting of glycerol, sorbitol, xylitol, mannitol, erythritol, maltitol, glucose, glucitol, ribulose, pentaerythritol, trimethylolpropane.

In one embodiment, the compound of formula 1 is selected from the group consisting of

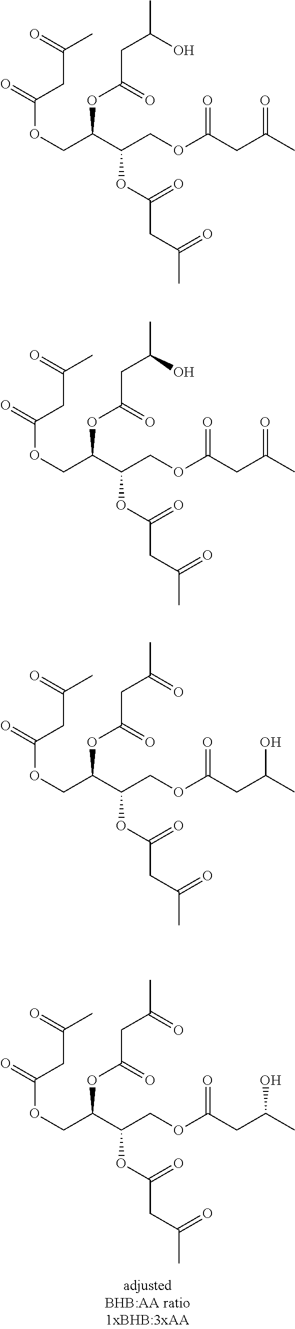 [SMILES: CC(=O)CC(=O)OC[C@H](OC(=O)CC(C)=O)[C@@H](COC(=O)CC(C)=O)OC(=O)CC(C)O ; CC(=O)CC(=O)OC[C@H](OC(=O)CC(C)=O)[C@@H](COC(=O)CC(C)=O)OC(=O)C[C@@H](C)O ; CC(=O)CC(=O)OC[C@@H](OC(=O)CC(C)=O)[C@H](COC(=O)CC(C)O)OC(=O)CC(C)=O ; CC(=O)CC(=O)OC[C@@H](OC(=O)CC(C)=O)[C@H](COC(=O)C[C@@H](C)O)OC(=O)CC(C)=O]

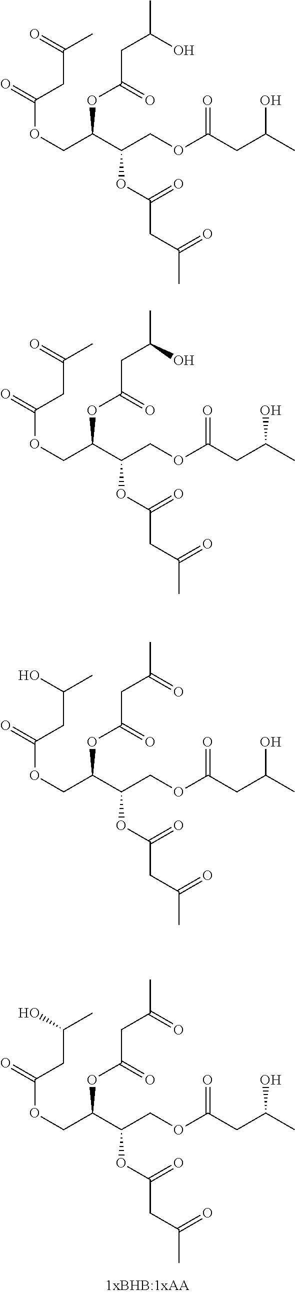

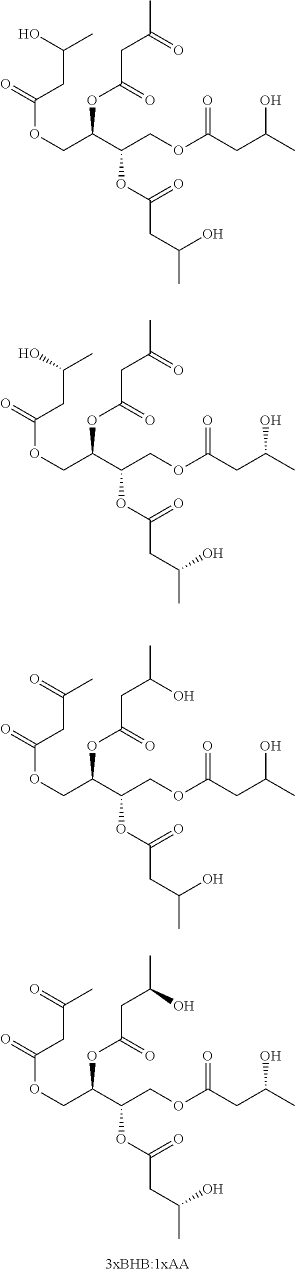

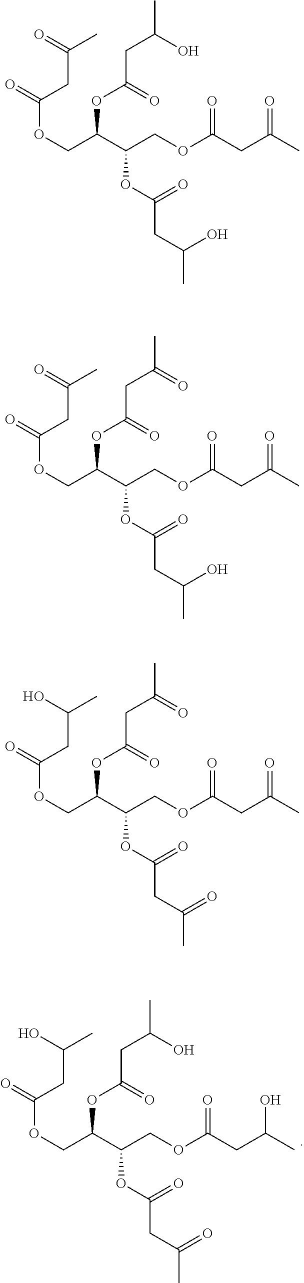 [SMILES: CC(=O)CC(=O)OC[C@H](OC(=O)CC(C)O)[C@@H](COC(=O)CC(C)=O)OC(=O)CC(C)O ; CC(=O)CC(=O)OC[C@H](OC(=O)CC(C)O)[C@@H](COC(=O)CC(C)=O)OC(=O)CC(C)=O ; CC(=O)CC(=O)OC[C@H](OC(=O)CC(C)=O)[C@@H](COC(=O)CC(C)O)OC(=O)CC(C)=O ; CC(=O)CC(=O)O[C@@H](COC(=O)CC(C)O)[C@@H](COC(=O)CC(C)O)OC(=O)CC(C)O]

In one embodiment, the compound of formula 1 is selected from the group consisting of

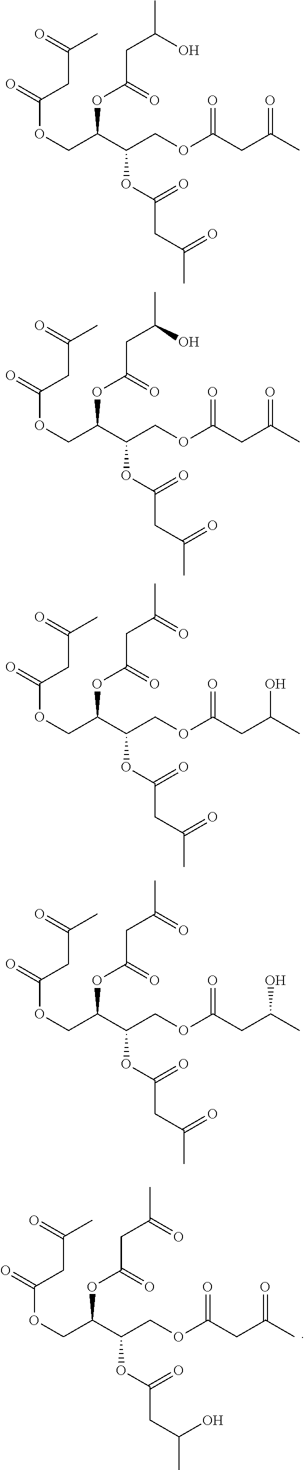

In one embodiment, the compound of formula 1 is selected from the group consisting of

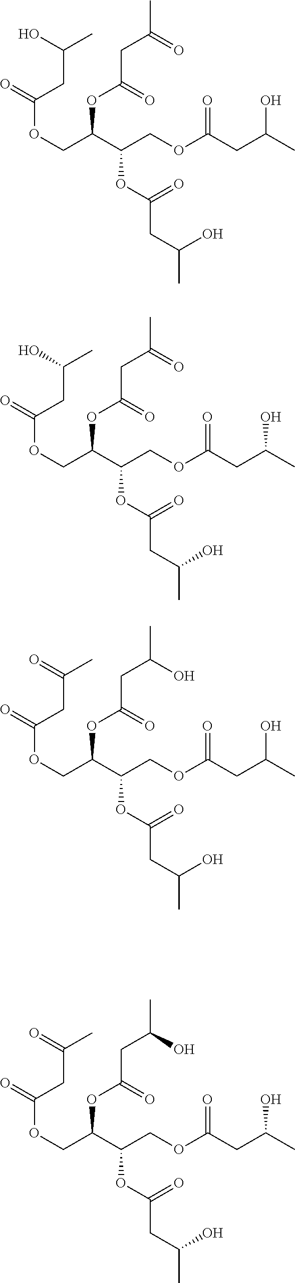

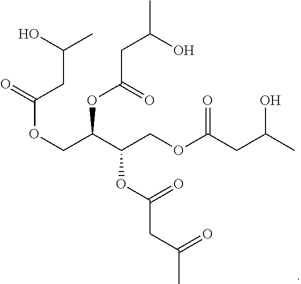 [SMILES: CC(=O)CC(=O)O[C@@H](COC(=O)CC(C)O)[C@@H](COC(=O)CC(C)O)OC(=O)CC(C)O]

In one embodiment, in the compound according to formula 1, all β-hydroxyl butyric acid ester units are either D-configured or L-configured. In another embodiment, all β-hydroxyl butyric acid ester units are present in the compound according to formula 1 as a non-racemic mixture of D- and L-configurations.

In one embodiment, the compound according to formula 1 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 1, all β-hydroxyl butyric acid ester units are either R-configured or S-configured. In another embodiment, all β-hydroxyl butyric acid ester units are present in the compound according to formula 1 as a non-racemic mixture of R- and S-configurations.

In one embodiment, the compound according to formula 1 contains more R-configured β-hydroxyl butyric acid ester units than S-configured β-hydroxyl butyric acid ester units. Preferably all β-hydroxyl butyric acid ester units are in R-configuration.

In another aspect, the present invention provides a process for the preparation of a compound of formula 1

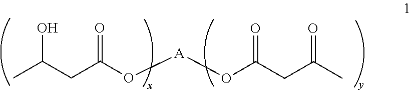

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) reacting an organic polyol of formula 2 with diketene 3 resulting in the formation of a compound according to formula 4;
   and

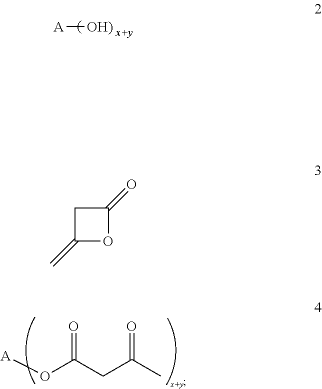

(ii) partially hydrogenating the compound of formula 4 by reacting the compound of formula 4 with hydrogen in the presence of a catalyst resulting in the formation of the compound according to formula 1.

In another aspect, the present invention provides a process for the preparation of a compound of formula 1

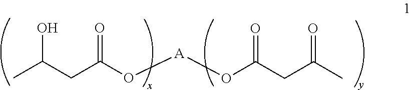

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) partially reacting an organic polyol of formula 2 with diketene 3 resulting in the formation of a compound according to formula 5;

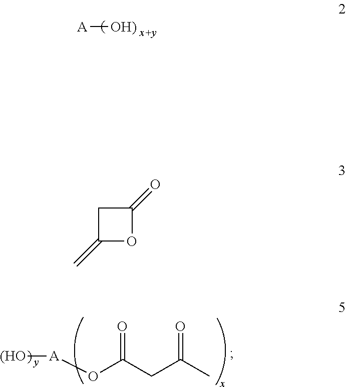

(ii) reacting the compound of formula 5 with hydrogen in the presence of a catalyst resulting in the formation of the compound according to formula 6

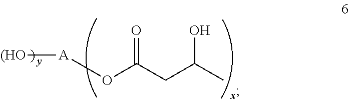

and
(iii) partially reacting the compound of formula 6 with diketene 3 resulting in the formation of the compound according to formula 1.

All embodiments and preferred embodiments disclosed above with respect to the compound of formula 1 likewise apply for the processes of preparing a compound of formula 1.

The inventors surprisingly found that the processes according to the present invention for the preparation of compounds according to formula 1 achieve significantly improved atom economy and cost efficiency per unit of acetoacetate if diketene 3 is employed directly in this reaction. Moreover, the inventive processes for the preparation of compounds according to formula 1 provide excellent processes for providing polyalcohol esters comprising both BHB and AA units in one molecule.

In one embodiment, reaction step (i) is performed in the presence of an organic amine catalyst. In one embodiment, reaction step (iii) is performed in the presence of an organic amine catalyst.

Suitable organic amine catalysts include tertiary amines. Preferably, the organic amine catalyst is 1,4-diazabicyclo[2.2.2]octane (DABCO).

In step (ii) a compound of formula 4 or a compound of formula 5 is reacted with hydrogen in the presence of a catalyst to reduce an AA unit to a BHB unit. In one embodiment, reaction step (ii) is performed in the presence of a metal-based catalyst. Preferably, the metal-based catalyst is a Ni-based catalyst, a Pd-based catalyst, a Pt-based catalyst, a Ru-based catalyst, a Co-based catalyst, an Ir-based catalyst, or an Rh-based catalyst.

In one embodiment, reaction step (ii) is performed in presence of a chiral ligand capable of forming complexes with the metal-based catalyst. Preferred chiral ligand are selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino) butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).

Preferably, reaction step (ii) is performed in the presence of a Ru-based catalyst. A preferred Ru-based catalyst is a Ruthenium oxide catalyst such as RuO₂. Further preferred Ru-based catalysts include Ru/C, RuAl₂O₃, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)-C3-TunePhos)(p-cymene)ruthenium(II)]chloride.

Depending on the type of the organic polyol, the process for the preparation of a compound of formula 1 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 1 is performed without a solvent. In another embodiment, the process for the preparation of a compound of formula 1 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, acetonitrile, toluene, chloroform, 1,4-dioxan,, or o/m/p-xylene. Preferably, the organic solvent is ethyl acetate.

In one embodiment, in the process for the preparation of a compound of formula 1, reaction step (i) is performed at temperature of 0-100° C. Preferably, reaction step (i) is performed at temperature of 15-70° C. Additionally, the reaction temperature of reaction step (i) may be maintained at 20-70° C. after complete addition of diketene 3.

In one embodiment, during reaction step (i) diketene 3 is slowly added over a period of 1-10 h, e.g. dropwise, to the reaction mixture, to avoid the formation of side products.

In one embodiment, in the process for the preparation of a compound of formula 1, reaction step (iii) is performed at temperature of 0-100° C. Preferably, reaction step (iii) is performed at temperature of 15-70° C. Additionally, the reaction temperature of reaction step (iii) may be maintained at 20-70° C. after complete addition of diketene 3.

In one embodiment, during reaction step (iii) diketene 3 is slowly added over a period of 1-10 h, e.g. dropwise, to the reaction mixture, to avoid the formation of side products.

In one embodiment reaction step (ii) is performed in a closed vessel under hydrogen pressure. Preferably, reaction step (ii) is performed at 5-30 bar hydrogen pressure and even more preferably at 10-20 bar hydrogen pressure.

In one embodiment, reaction step (ii) is performed at a temperature of 20-90° C. Preferably, reaction step (ii) is performed at a temperature of 50-70° C. and more preferably, reaction step (ii) is performed at a temperature of about 60° C.

In one embodiment, reaction step (ii) is stirred at 800-1200 rpm so as to ensure sufficient hydrogen diffusion into the reaction mixture.

An exemplary process for the preparation of compounds according to formula 1 is provided in the following reaction scheme:

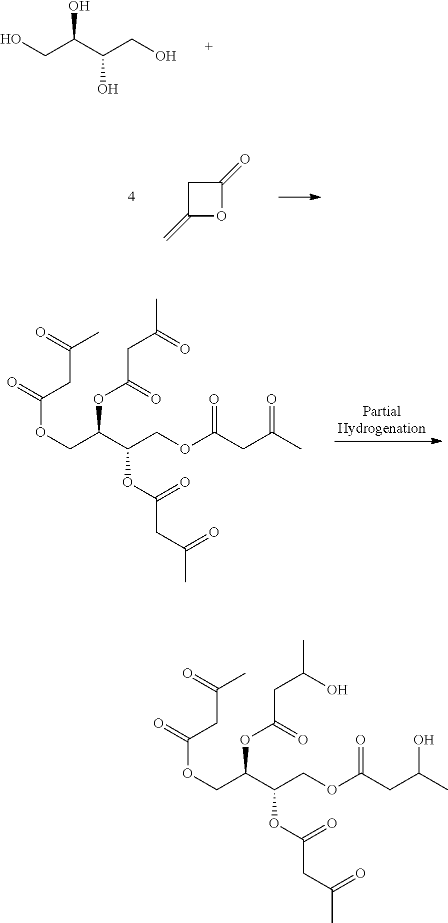

An exemplary process for the preparation of compounds according to formula 1 is provided in the following reaction scheme:

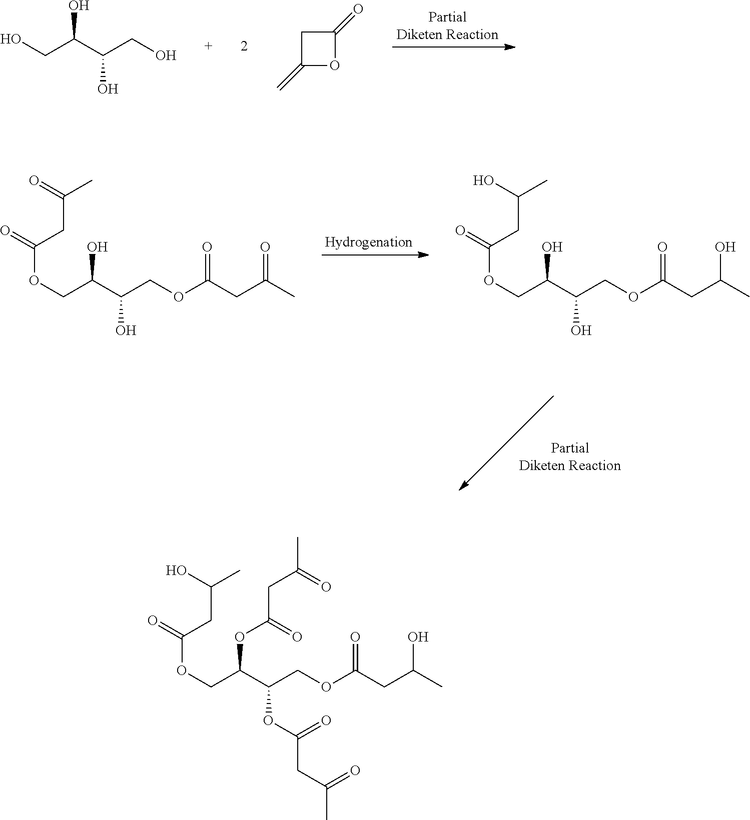

In another aspect, the present invention provides a compound of formula 6

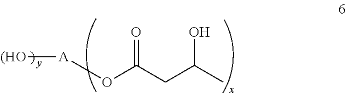

wherein
A is derived from an organic polyol with at least 3 hydroxyl groups,
x is at least 1,
y is at least 1, and
x+y is from 3 to the number of hydroxyl groups of the initial organic polyol A.

In one embodiment, the organic polyol has from 3 to 10 hydroxyl groups. Preferably, the organic polyol has from 3 to 8 hydroxyl groups, such as from 3 to 7 hydroxyl groups, from 3 to 6 hydroxyl groups, more preferably from 3 to 5 hydroxyl groups, or from 3 to 4 hydroxyl groups.

In one embodiment, x is at least 2, at least 3, or at least 4. In one embodiment, x is 1, 2, 3, 4, 5, or 6. Preferably, x is 1, 2, 3, or 4.

In one embodiment, y is at least 2, at least 3, or at least 4. In one embodiment, y is 1, 2, 3, 4, 5, or 6. Preferably, y is 1, 2, 3, or 4.

In one embodiment, x is 1 and y is 3. In one embodiment, x is 2 and y is 2. In one embodiment, x is 3 and y is 1.

In one embodiment, x+y is from 3 to 10, such as from 3 to 8, from 3 to 7, from 3 to 6, from 3 to 5 or from 3 to 4. Accordingly, x+y may be 3, 4, 5, 6, 7, 8, 9, or 10, preferably 3, 4, 5, or 6, more preferably, x+y may be 3, 4, or 5.

In one embodiment, the organic polyol is a linear, branched, or cyclic organic compound with 2 to 18 carbon atoms having at least three hydroxyl groups. In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

In another preferred embodiment, the linear or branched C₂₋₁₂ alkyl substituted with at least 3 hydroxyl groups is selected from the group consisting of trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

Preferably, the C₃₋₈ cycloalkyl substituted with at least 3 hydroxyl groups is selected from the group consisting of cyclopentanetriol, cyclohexanetriol, cyclopentanetetrol, cyclohexanetetrol.

In one embodiment, the organic polyol is selected from the group consisting of monosaccharides, sugar alcohols, and sugar acids.

In one embodiment, the monosaccharide is selected from tetroses, pentoses, hexoses, heptoses. Preferably, the monosaccharide is selected from aldotetroses, ketotetroses, aldopentoses, ketopentoses, aldohexosen, ketohexoses, aldoheptoses, ketoheptoses.

In one embodiment, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.

In one embodiment, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.

In one embodiment, the sugar acid is selected from aldonic acids, ulosonic acids, uronic acids, aldaric acids. Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.

In one embodiment, the organic polyol is selected from the group consisting of glycerol, sorbitol, xylitol, mannitol, erythritol, maltitol, glucose, glucitol, ribulose, pentaerythritol, trimethylolpropane.

In one embodiment, the compound of formula 6 is selected from the group consisting of

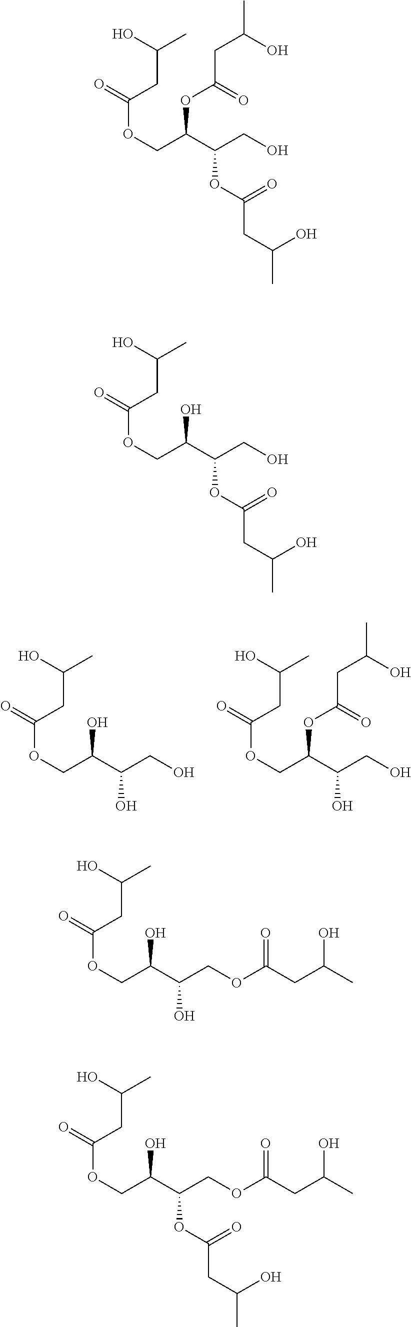 [SMILES: CC(O)CC(=O)OC[C@@H](OC(=O)CC(C)O)[C@H](CO)OC(=O)CC(C)O ; CC(O)CC(=O)OC[C@@H](O)[C@H](CO)OC(=O)CC(C)O ; CC(O)CC(=O)OC[C@@H](O)[C@@H](O)CO ; CC(O)CC(=O)OC[C@@H](OC(=O)CC(C)O)[C@@H](O)CO ; CC(O)CC(=O)OC[C@@H](O)[C@@H](O)COC(=O)CC(C)O ; CC(O)CC(=O)OC[C@@H](O)[C@H](COC(=O)CC(C)O)OC(=O)CC(C)O]

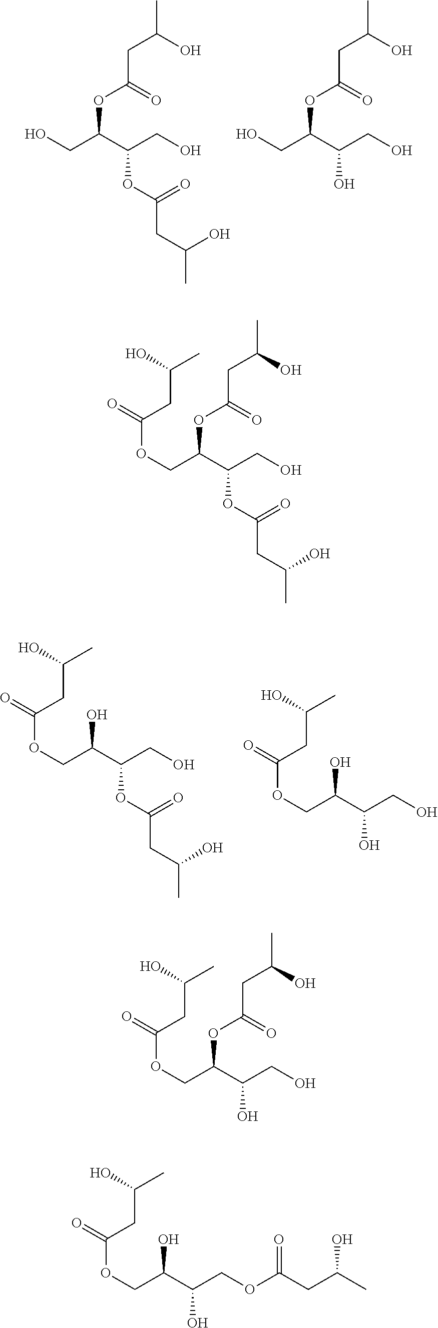

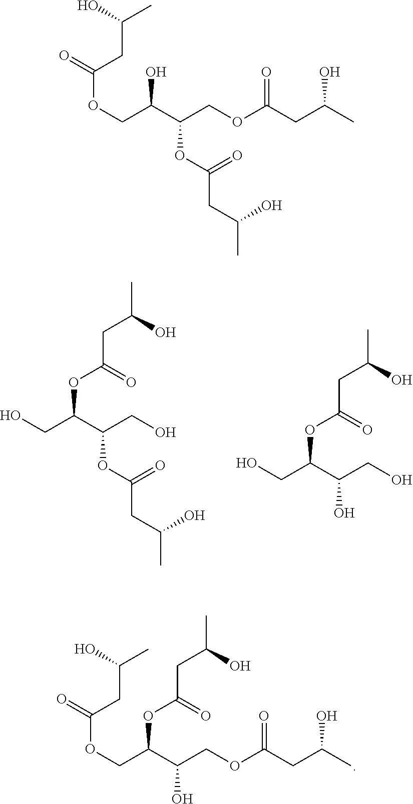 [SMILES: C[C@@H](O)CC(=O)OC[C@@H](O)[C@H](COC(=O)C[C@@H](C)O)OC(=O)C[C@@H](C)O ; C[C@@H](O)CC(=O)O[C@@H](CO)[C@@H](CO)OC(=O)C[C@@H](C)O ; C[C@@H](O)CC(=O)O[C@H](CO)[C@@H](O)CO ; C[C@@H](O)CC(=O)OC[C@H](O)[C@@H](COC(=O)C[C@@H](C)O)OC(=O)C[C@@H](C)O]

In one embodiment, in the compound according to formula 6, all β-hydroxyl butyric acid ester units are either D-configured or L-configured. In another embodiment, all β-hydroxyl butyric acid ester units are present in the compound according to formula 6 as a non-racemic mixture of D- and L-configurations.

In one embodiment, the compound according to formula 6 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 6, all β-hydroxyl butyric acid ester units are either R-configured or S-configured. In another embodiment, all β-hydroxyl butyric acid ester units are present in the compound according to formula 6 as a non-racemic mixture of R- and S-configurations.

In one embodiment, the compound according to formula 6 contains more R-configured β-hydroxyl butyric acid ester units than S-configured β-hydroxyl butyric acid ester units. Preferably all β-hydroxyl butyric acid ester units are in R-configuration.

In another aspect, the present invention provides a process for the preparation of a compound of formula 6

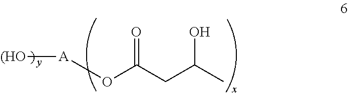

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) partially reacting an organic polyol of formula 2 with diketene 3 resulting in the formation of a compound according to formula 5;

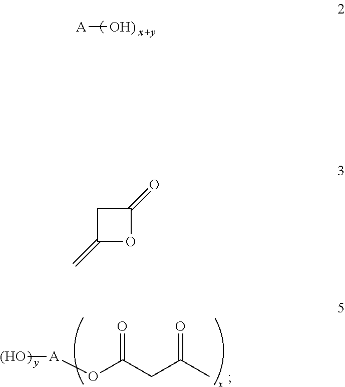

and
(ii) reacting the compound of formula 5 with hydrogen in the presence of a catalyst resulting in the formation of the compound according to formula 6

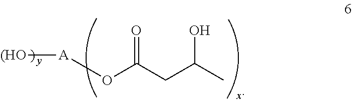

All embodiments and preferred embodiments disclosed above with respect to the compound of formula 6 likewise apply for the processes of preparing a compound of formula 6.

In one embodiment, reaction step (i) is performed in the presence of an organic amine catalyst.

Suitable organic amine catalysts include tertiary amines. Preferably, the organic amine catalyst is 1,4-diazabicyclo[2.2.2]octane (DABCO).

In step (ii) a compound of formula 5 is reacted with hydrogen in the presence of a catalyst to reduce an AA unit to a BHB unit. In one embodiment, reaction step (ii) is performed in the presence of a metal-based catalyst. Preferably, the metal-based catalyst is a Ni-based catalyst, a Pd-based catalyst, a Pt-based catalyst, a Ru-based catalyst, a Co-based catalyst, an Ir-based catalyst, or an Rh-based catalyst.

In one embodiment, reaction step (ii) is performed in presence of a chiral ligand capable of forming complexes with the metal-based catalyst. Preferred chiral ligand are selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino) butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).

Preferably, reaction step (ii) is performed in the presence of a Ru-based catalyst. A preferred Ru-based catalyst is a Ruthenium oxide catalyst such as RuO₂. Further preferred Ru-based catalysts include Ru/C, RuAl₂O₃, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)-C3-TunePhos)(p-cymene)ruthenium(II)]chloride.

Depending on the type of the organic polyol, the process for the preparation of a compound of formula 6 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 6 is performed without a solvent. In another embodiment, the process for the preparation of a compound of formula 6 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, acetonitrile, toluene, chloroform, 1,4-dioxan,, or o/m/p-xylene. Preferably, the organic solvent is ethyl acetate.

In one embodiment, in the process for the preparation of a compound of formula 6, reaction step (i) is performed at temperature of 0-100° C. Preferably, reaction step (i) is performed at temperature of 15-70° C. Additionally, the reaction temperature of reaction step (i) may be maintained at 20-70° C. after complete addition of diketene 3.

In one embodiment, during reaction step (i) diketene 3 is slowly added over a period of 1-10, e.g. dropwise, to the reaction mixture, to avoid the formation of side products.

In one embodiment reaction step (ii) is performed in a closed vessel under hydrogen pressure. Preferably, reaction step (ii) is performed at 5-30 bar hydrogen pressure and even more preferably at 10-20 bar hydrogen pressure.

In one embodiment, reaction step (ii) is performed at a temperature of 20-90° C. Preferably, reaction step (ii) is performed at a temperature of 50-70° C. and more preferably, reaction step (ii) is performed at a temperature of about 60° C.

In one embodiment, reaction step (ii) is stirred at 800-1200 rpm so as to ensure sufficient hydrogen diffusion into the reaction mixture.

In another aspect, the present invention provides a compound of formula 7

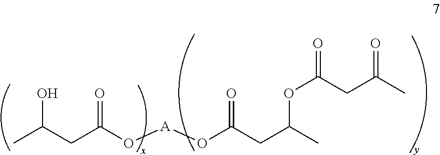

wherein
A is derived from an organic polyol with at least 3 hydroxyl groups,
x is at least 1,
y is at least 1, and
x+y is from 3 to the number of hydroxyl groups of the initial organic polyol A.

In one embodiment, the organic polyol has from 3 to 10 hydroxyl groups. Preferably, the organic polyol has from 3 to 8 hydroxyl groups, such as from 3 to 7 hydroxyl groups, from 3 to 6 hydroxyl groups, more preferably from 3 to 5 hydroxyl groups, or from 3 to 4 hydroxyl groups.

In one embodiment, x is at least 2, at least 3, or at least 4. In one embodiment, x is 1, 2, 3, 4, 5, or 6. Preferably, x is 1, 2, 3, or 4.

In one embodiment, y is at least 2, at least 3, or at least 4. In one embodiment, y is 1, 2, 3, 4, 5, or 6. Preferably, y is 1, 2, 3, or 4.

In one embodiment, x is 1 and y is 3. In one embodiment, x is 2 and y is 2. In one embodiment, x is 3 and y is 1.

In one embodiment, x+y is from 3 to 10, such as from 3 to 8, from 3 to 7, from 3 to 6, from 3 to 5 or from 3 to 4. Accordingly, x+y may be 3, 4, 5, 6, 7, 8, 9, or 10, preferably 3, 4, 5, or 6, more preferably, x+y may be 3, 4, or 5.

In one embodiment, x+y is equal to the number of hydroxyl groups of the initial polyol A. In one embodiment, x+y is less than the number of hydroxyl groups of the initial polyol A.

In one embodiment, the organic polyol is a linear, branched, or cyclic organic compound with 2 to 18 carbon atoms having at least three hydroxyl groups.

In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

In another preferred embodiment, the linear or branched C₂₋₁₂ alkyl substituted with at least 3 hydroxyl groups is selected from the group consisting of trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

Preferably, the C₃₋₈ cycloalkyl substituted with at least 3 hydroxyl groups is selected from the group consisting of cyclopentanetriol, cyclohexanetriol, cyclopentanetetrol, cyclohexanetetrol.

In one embodiment, the organic polyol is selected from the group consisting of monosaccharides, sugar alcohols, and sugar acids.

In one embodiment, the monosaccharide is selected from tetroses, pentoses, hexoses, heptoses. Preferably, the monosaccharide is selected from aldotetroses, ketotetroses, aldopentoses, ketopentoses, aldohexosen, ketohexoses, aldoheptoses, ketoheptoses.

In one embodiment, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.

In one embodiment, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.

In one embodiment, the sugar acid is selected from aldonic acids, ulosonic acids, uronic acids, aldaric acids. Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.

In one embodiment, the organic polyol is selected from the group consisting of glycerol, sorbitol, xylitol, mannitol, erythritol, maltitol, glucose, glucitol, ribulose, pentaerythritol, trimethylolpropane.

In one embodiment, the compound of formula 7 is selected from the group consisting of

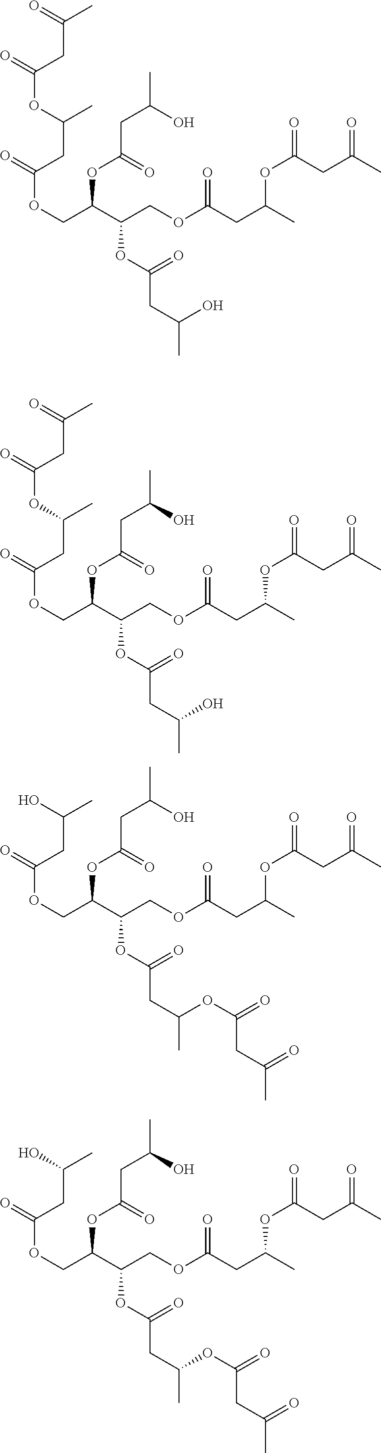

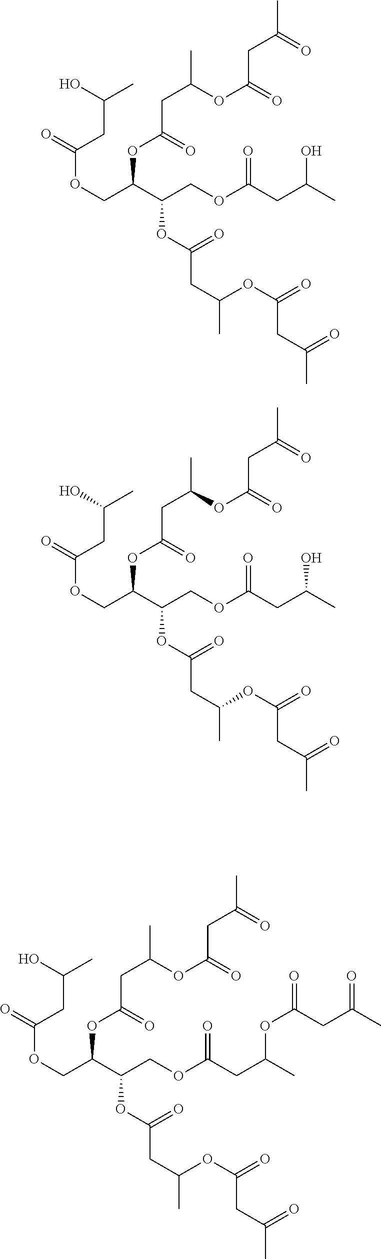

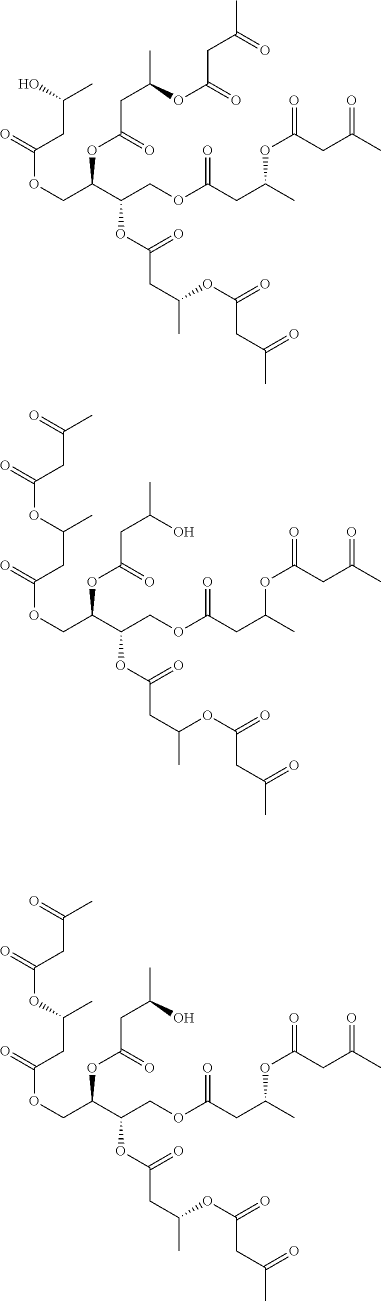 [SMILES: CC(=O)CC(=O)O[C@H](C)CC(=O)OC[C@H](OC(=O)C[C@@H](C)OC(=O)CC(C)=O)[C@@H](COC(=O)C[C@@H](C)O)OC(=O)C[C@@H](C)OC(=O)CC(C)=O ; CC(=O)CC(=O)OC(C)CC(=O)OC[C@H](OC(=O)CC(C)OC(=O)CC(C)=O)[C@@H](COC(=O)CC(C)OC(=O)CC(C)=O)OC(=O)CC(C)O ; CC(=O)CC(=O)O[C@H](C)CC(=O)OC[C@H](OC(=O)C[C@@H](C)OC(=O)CC(C)=O)[C@@H](COC(=O)C[C@@H](C)OC(=O)CC(C)=O)OC(=O)C[C@@H](C)O]

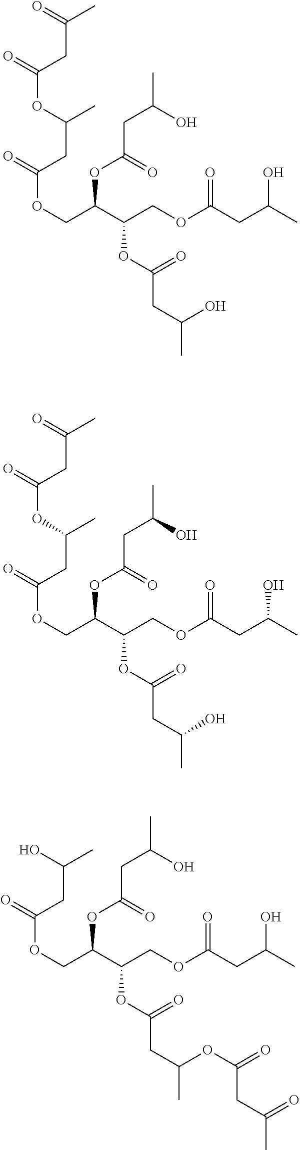 [SMILES: CC(=O)CC(=O)OC(C)CC(=O)OC[C@@H](OC(=O)CC(C)O)[C@H](COC(=O)CC(C)O)OC(=O)CC(C)O ; CC(=O)CC(=O)O[C@H](C)CC(=O)OC[C@@H](OC(=O)C[C@@H](C)O)[C@H](COC(=O)C[C@@H](C)O)OC(=O)C[C@@H](C)O ; CC(=O)CC(=O)OC(C)CC(=O)O[C@@H](COC(=O)CC(C)O)[C@@H](COC(=O)CC(C)O)OC(=O)CC(C)O]

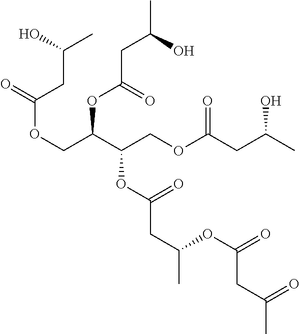 [SMILES: CC(=O)CC(=O)O[C@H](C)CC(=O)O[C@@H](COC(=O)C[C@@H](C)O)[C@@H](COC(=O)C[C@@H](C)O)OC(=O)C[C@@H](C)O]

These exemplary compounds comprise AA residues and BHB residues in ratios of 1:2, 1:4 or 3:4.

In one embodiment, in the compound according to formula 7, all β-hydroxyl butyric acid ester units are either D-configured or L-configured. In another embodiment, all β-hydroxyl butyric acid ester units are present in the compound according to formula 7 as a non-racemic mixture of D- and L-configurations.

In one embodiment, the compound according to formula 7 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 7, all β-hydroxyl butyric acid ester units are either R-configured or S-configured. In another embodiment, all β-hydroxyl butyric acid ester units are present in the compound according to formula 7 as a non-racemic mixture of R- and S-configurations.

In one embodiment, the compound according to formula 7 contains more R-configured β-hydroxyl butyric acid ester units than S-configured β-hydroxyl butyric acid ester units. Preferably all β-hydroxyl butyric acid ester units are in R-configuration.

In another aspect, the present invention provides a process for the preparation of a compound of formula 7

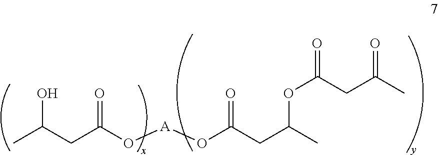

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) reacting an organic polyol of formula 2 with diketene 3 resulting in the formation of a compound according to formula 4;

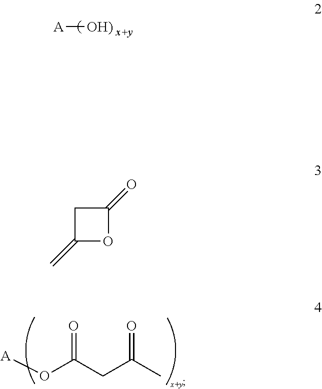

(ii) reacting the compound of formula 4 with hydrogen in the presence of a catalyst resulting in the formation of a compound according to formula 8;
   and

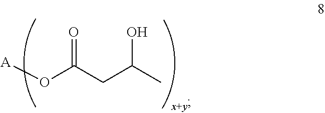

(iii) partially reacting the compound according to formula 8 with diketene 3 resulting in the formation of a compound according to formula 7.

All embodiments and preferred embodiments disclosed above with respect to the compound of formula 7 likewise apply for the process of preparing a compound of formula 7.

The inventors surprisingly found that the process according to the present invention for the preparation of compounds according to formula 7 achieve significantly improved atom economy and cost efficiency per unit of acetoacetate if diketene 3 is employed directly in this reaction. Moreover, the inventive process for the preparation of compounds according to formula 7 provides an excellent process for providing polyalcohol esters comprising both BHB and AA units in one molecule.

In one embodiment, reaction step (i) is performed in the presence of an organic amine catalyst. In one embodiment, reaction step (iii) is performed in the presence of an organic amine catalyst.

Suitable organic amine catalysts include tertiary amines. Preferably, the organic amine catalyst is 1,4-diazabicyclo[2.2.2]octane (DABCO).

In step (ii) a compound of formula 4 is reacted with hydrogen in the presence of a catalyst to reduce an AA unit to a BHB unit. In one embodiment, reaction step (ii) is performed in the presence of a metal-based catalyst. Preferably, the metal-based catalyst is a Ni-based catalyst, a Pd-based catalyst, a Pt-based catalyst, a Ru-based catalyst, a Co-based catalyst, an Ir-based catalyst, or an Rh-based catalyst.

In one embodiment, reaction step (ii) is performed in presence of a chiral ligand capable of forming complexes with the metal-based catalyst. Preferred chiral ligand are selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino) butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).

Preferably, reaction step (ii) is performed in the presence of a Ru-based catalyst. A preferred Ru-based catalyst is a Ruthenium oxide catalyst such as RuO₂. Further preferred Ru-based catalysts include Ru/C, RuAl₂O₃, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)-C3-TunePhos)(p-cymene)ruthenium(II)]chloride.

Depending on the type of the organic polyol, the process for the preparation of a compound of formula 1 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 7 is performed without a solvent. In another embodiment, the process for the preparation of a compound of formula 7 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, acetonitrile, toluene, chloroform, 1,4-dioxan, or o/m/p-xylene. Preferably, the organic solvent is ethyl acetate.

In one embodiment, in the process for the preparation of a compound of formula 7, reaction step (i) is performed at temperature of 0-100° C. Preferably, reaction step (i) is performed at temperature of 15-70° C. Additionally, the reaction temperature of reaction step (i) may be maintained at 20-70° C. after complete addition of diketene 3.

In one embodiment, during reaction step (i) diketene 3 is slowly added over a period of 1-10 h, e.g. dropwise, to the reaction mixture, to avoid the formation of side products.

In one embodiment, in the process for the preparation of a compound of formula 7, reaction step (iii) is performed at temperature of 0-100° C. Preferably, reaction step (iii) is performed at temperature of 15-70° C. Additionally, the reaction temperature of reaction step (iii) may be maintained at 20-70° C. after complete addition of diketene 3.

In one embodiment, during reaction step (iii) diketene 3 is slowly added over a period of 1-10 h, e.g. dropwise, to the reaction mixture, to avoid the formation of side products.

In one embodiment reaction step (ii) is performed in a closed vessel under hydrogen pressure. Preferably, reaction step (ii) is performed at 5-30 bar hydrogen pressure and even more preferably at 10-20 bar hydrogen pressure.

In one embodiment, reaction step (ii) is performed at a temperature of 20-90° C. Preferably, reaction step (ii) is performed at a temperature of 50-70° C. and more preferably, reaction step (ii) is performed at a temperature of about 60° C.

In one embodiment, reaction step (ii) is stirred at 800-1200 rpm so as to ensure sufficient hydrogen diffusion into the reaction mixture.

An exemplary process for the preparation of compounds according to formula 7 is provided in the following reaction scheme:

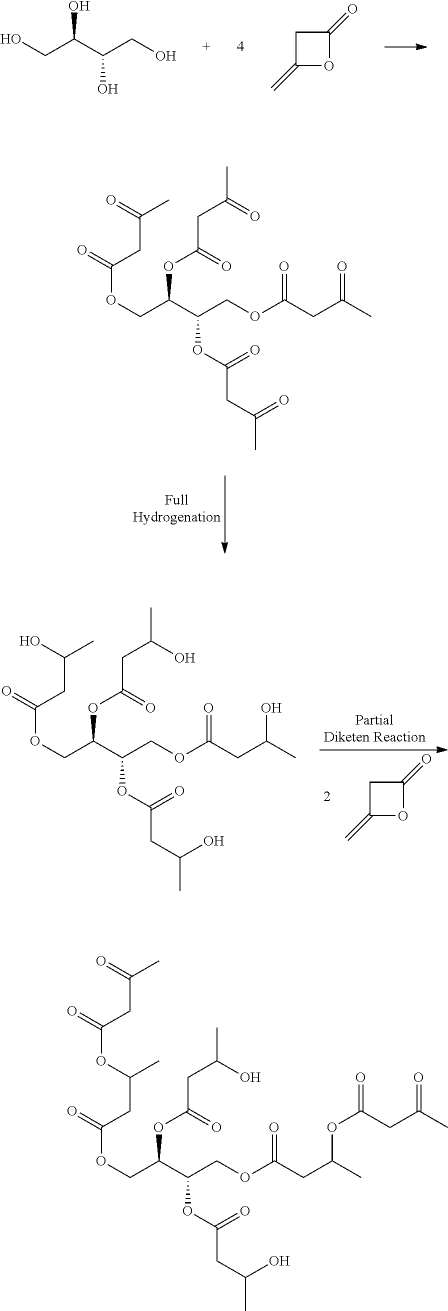

In another aspect, the present invention provides a compound of formula 9

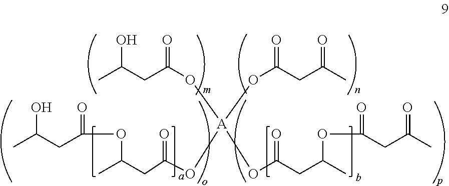

wherein
A is derived from an organic polyol with at least 3 hydroxyl groups,
m is 0, 1, 2, 3, 4, or 5,
n is 0, 1, 2, 3, 4, or 5,
o is 0, 1, 2, 3, 4, or 5,
p is 0, 1, 2, 3, 4, or 5,
at least two of m, n, o, and p are not 0,
m+n+o+p is from 2 to the number of hydroxyl groups of the initial organic polyol A,
a is 1-10, and
b is 1-10.

In one embodiment, the organic polyol has from 3 to 10 hydroxyl groups. Preferably, the organic polyol has from 3 to 8 hydroxyl groups, such as from 3 to 7 hydroxyl groups, from 3 to 6 hydroxyl groups, from 3 to 5 hydroxyl groups, or from 3 to 4 hydroxyl groups.

In one embodiment, m is 0, 1, 2, or 3.

In one embodiment, n is 0, 1, 2, or 3.

In one embodiment, o is 0, 1, 2, or 3.

In one embodiment, p is 0, 1, 2, or 3.

In one embodiment, m is 0, 1, 2, or 3; n is 0, 1, 2, or 3; o is 0, 1, 2, or 3; and p is 0, 1, 2, or 3.

In one embodiment, two of m, n, o, and p are not 0. In one embodiment, n and p are 2 and m and o are 0.

In one embodiment, at least three of m, n, o, and p are not 0. In one embodiment, three of m, n, o, and p are not 0. In one embodiment, m, n, and p are not 0. In one embodiment, m, n, and p are 1 and o is 0.

In one embodiment, m+n+o+p is from 3 to the number of hydroxyl groups of the initial organic polyol A

In one embodiment, m+n+o+p is equal to the number of hydroxyl groups of the initial polyol A.

In one embodiment, m+n+o+p is less than the number of hydroxyl groups of the initial polyol A.

In one embodiment, m+n+o+p is from 2 to 10, such as from 3 to 10, from 3 to 8, from 3 to 7, from 3 to 6, from 3 to 5 or from 3 to 4. Accordingly, m+n+o+p may be 3, 4, 5, 6, 7, 8, 9, or 10, preferably 3, 4, 5, or 6, more preferably 3, 4, or 5.

In one embodiment, a is 1-9, such as 1-8, such as 1-7, such as 1-6, such as 1-5, such as 1-4, such as 1-3, or such as 1-2, preferably wherein a is 1 or 2, more preferably wherein a is 1.

In one embodiment, b is 1-9, such as 1-8, such as 1-7, such as 1-6, such as 1-5, such as 1-4, such as 1-3, or such as 1-2, preferably wherein b is 1 or 2, more preferably wherein b is 1.

In one embodiment, the number of AA residues differs from the number of BHB residues, i.e., n+p+a+b+m+o. In other words, the ratio of (n+p):(a+b+m+o) is not 1:1.

In one embodiment, the organic polyol is a linear, branched, or cyclic organic compound with 2 to 18 carbon atoms having at least three hydroxyl groups.

In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

In another preferred embodiment, the linear or branched C₂₋₁₂ alkyl substituted with at least 3 hydroxyl groups is selected from the group consisting of trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

Preferably, the C₃₋₈ cycloalkyl substituted with at least 3 hydroxyl groups is selected from the group consisting of cyclopentanetriol, cyclohexanetriol, cyclopentanetetrol, cyclohexanetetrol.

In one embodiment, the organic polyol is selected from the group consisting of monosaccharides, sugar alcohols, and sugar acids.

Monosaccharides generally have the chemical formula CₙH₂ₙOₙ. Monosaccharides can be classified by the number x of carbon atoms they contain (CH₂O)ₓ: trioses (x=3), tetroses (x=4), pentoses (x=5), hexoses (x=6) and heptoses (x=7).

In one embodiment, the monosaccharide is selected from tetroses, pentoses, hexoses, heptoses. Preferably, the monosaccharide is selected from aldotetroses, ketotetroses, aldopentoses, ketopentoses, aldohexosen, ketohexoses, aldoheptoses, ketoheptoses.

In one embodiment, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.

Sugar alcohols (also called polyhydric alcohols, polyalcohols, alditols or glycitols) are organic compounds, typically derived from sugars, containing one hydroxyl group (—OH) attached to each carbon atom.

In one embodiment, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.

A sugar acid is generally a monosaccharide with a carboxyl group at one end or both ends of the carbon chain. Main classes of sugar acids include aldonic acids, ulosonic acids, uronic acids, and aldaric acids. In aldonic acids, the aldehyde group (—CHO) located at the initial end (position 1) of an aldose is oxidized. In ulosonic acids, the —CH₂(OH) group at the initial end of a 2-ketose is oxidized yielding an α-ketoacid. In uronic acids, the —CH₂(OH) group at the terminal end of an aldose or ketose is oxidized. In aldaric acids, both ends (—CHO and —CH₂(OH)) of an aldose are oxidized.

In one embodiment, the sugar acid is selected from aldonic acids, ulosonic acids, uronic acids, aldaric acids. Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.

In one embodiment, the organic polyol is selected from the group consisting of glycerol, sorbitol, xylitol, mannitol, erythritol, maltitol, glucose, glucitol, ribulose, pentaerythritol, trimethylolpropane.

In one embodiment, the compound of formula 9 is selected from the group consisting of

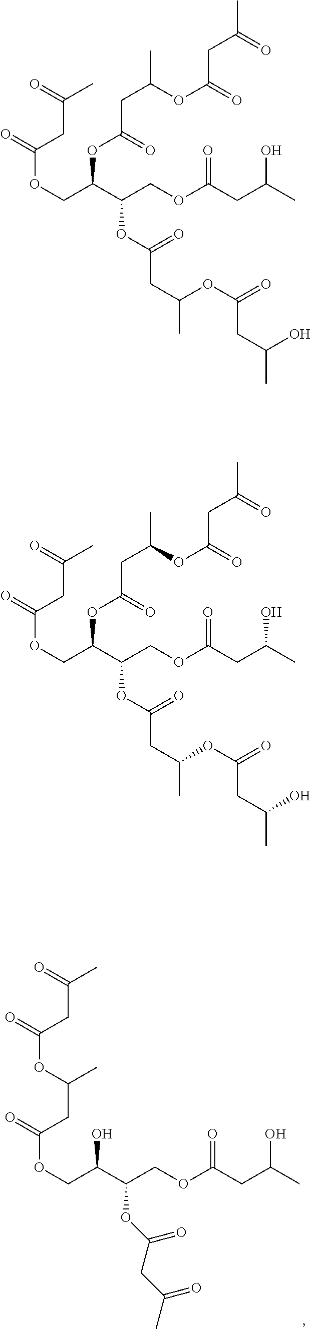 [SMILES: CC(=O)CC(=O)OC[C@@H](OC(=O)CC(C)OC(=O)CC(C)=O)[C@H](COC(=O)CC(C)O)OC(=O)CC(C)OC(=O)CC(C)O ; CC(=O)CC(=O)OC[C@@H](OC(=O)C[C@@H](C)OC(=O)CC(C)=O)[C@H](COC(=O)C[C@@H](C)O)OC(=O)C[C@@H](C)OC(=O)C[C@@H](C)O ; CC(=O)CC(=O)OC(C)CC(=O)OC[C@@H](O)[C@H](COC(=O)CC(C)O)OC(=O)CC(C)=O]

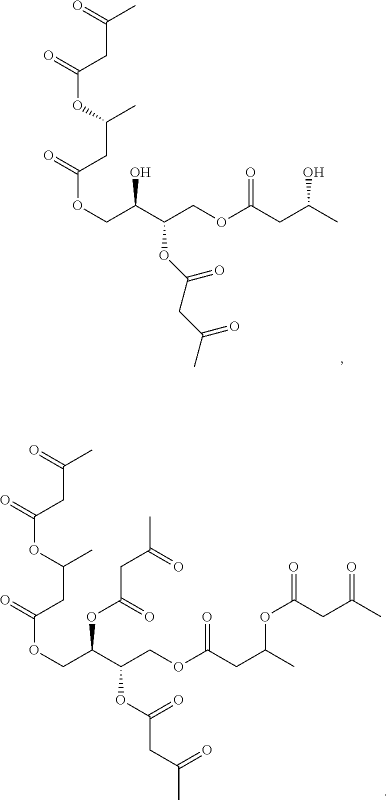 [SMILES: CC(=O)CC(=O)O[C@H](C)CC(=O)OC[C@@H](O)[C@H](COC(=O)C[C@@H](C)O)OC(=O)CC(C)=O ; CC(=O)CC(=O)OC(C)CC(=O)OC[C@H](OC(=O)CC(C)=O)[C@@H](COC(=O)CC(C)OC(=O)CC(C)=O)OC(=O)CC(C)=O]

In one embodiment, the compound of formula 9 is selected from the group consisting of

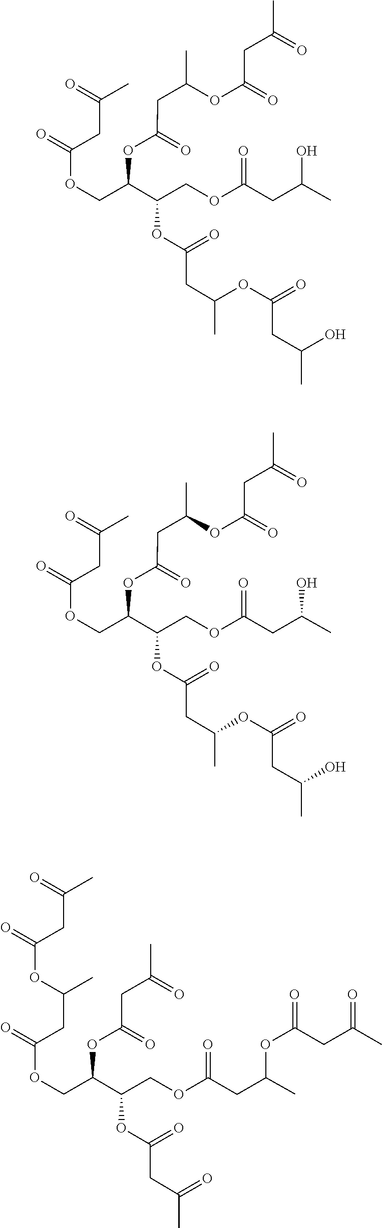 [SMILES: CC(=O)CC(=O)OC[C@@H](OC(=O)CC(C)OC(=O)CC(C)=O)[C@H](COC(=O)CC(C)O)OC(=O)CC(C)OC(=O)CC(C)O ; CC(=O)CC(=O)OC[C@@H](OC(=O)C[C@@H](C)OC(=O)CC(C)=O)[C@H](COC(=O)C[C@@H](C)O)OC(=O)C[C@@H](C)OC(=O)C[C@@H](C)O ; CC(=O)CC(=O)OC(C)CC(=O)OC[C@H](OC(=O)CC(C)=O)[C@@H](COC(=O)CC(C)OC(=O)CC(C)=O)OC(=O)CC(C)=O]

In one embodiment, in the compound according to formula 9, all β-hydroxyl butyric acid ester units are either D-configured or L-configured. In another embodiment, all β-hydroxyl butyric acid ester units are present in the compound according to formula 9 as a non-racemic mixture of D- and L-configurations.

In one embodiment, the compound according to formula 9 contains more D-configured β-hydroxyl butyric acid ester units than L-configured β-hydroxyl butyric acid ester units. Preferably all-hydroxyl butyric acid ester units are in D-configuration.

In one embodiment, in the compound according to formula 9, all β-hydroxyl butyric acid ester units are either R-configured or S-configured. In another embodiment, all-hydroxyl butyric acid ester units are present in the compound according to formula 9 as a non-racemic mixture of R- and S-configurations.

In one embodiment, the compound according to formula 9 contains more R-configured β-hydroxyl butyric acid ester units than S-configured β-hydroxyl butyric acid ester units. Preferably all β-hydroxyl butyric acid ester units are in R-configuration.

An exemplary process for the preparation of compounds according to formula 9 is provided in the following reaction scheme:

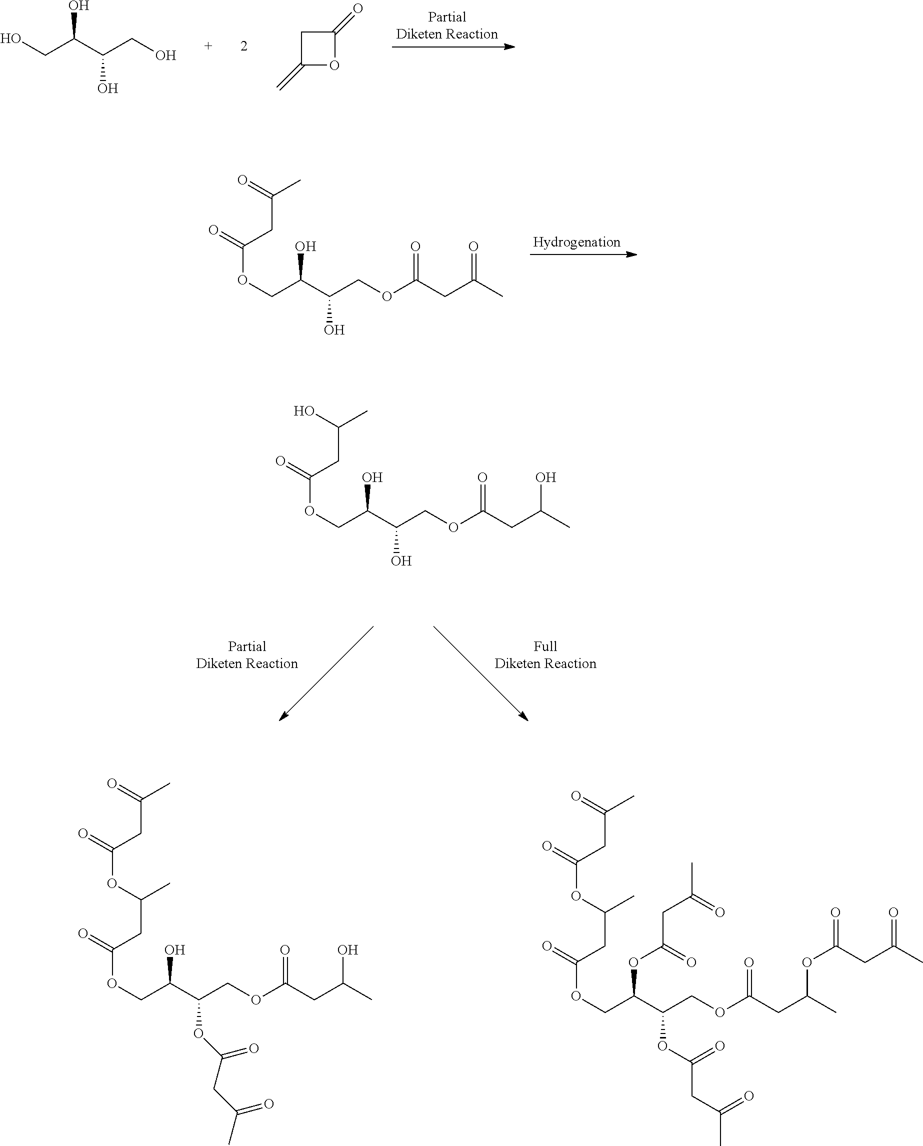

In another aspect, the present invention provides a compound of formula 10

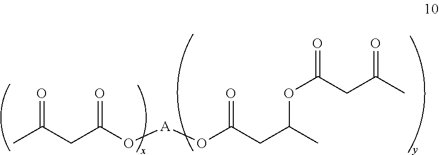

wherein
A is derived from an organic polyol with at least 3 hydroxyl groups,
x is at least 1,
y is at least 1, and
x+y is from 3 to the number of hydroxyl groups of the initial organic polyol A.

In one embodiment, the organic polyol has from 3 to 10 hydroxyl groups. Preferably, the organic polyol has from 3 to 8 hydroxyl groups, such as from 3 to 7 hydroxyl groups, from 3 to 6 hydroxyl groups, more preferably from 3 to 5 hydroxyl groups, or from 3 to 4 hydroxyl groups.

In one embodiment, x is at least 2, at least 3, or at least 4. In one embodiment, x is 1, 2, 3, 4, 5, or 6. Preferably, x is 1, 2, 3, or 4.

In one embodiment, y is at least 2, at least 3, or at least 4. In one embodiment, y is 1, 2, 3, 4, 5, or 6. Preferably, y is 1, 2, 3, or 4.

In one embodiment, x is 1 and y is 3. In one embodiment, x is 2 and y is 2. In one embodiment, x is 3 and y is 1.

In one embodiment, x+y is from 3 to 10, such as from 3 to 8, from 3 to 7, from 3 to 6, from 3 to 5 or from 3 to 4. Accordingly, x+y may be 3, 4, 5, 6, 7, 8, 9, or 10, preferably 3, 4, 5, or 6, more preferably 3, 4, or 5.

In one embodiment, x+y is equal to the number of hydroxyl groups of the initial polyol A. In one embodiment, x+y is less than the number of hydroxyl groups of the initial polyol A.

In one embodiment, the organic polyol is a linear, branched, or cyclic organic compound with 2 to 18 carbon atoms having at least three hydroxyl groups.

In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

In another preferred embodiment, the linear or branched C₂₋₁₂ alkyl substituted with at least 3 hydroxyl groups is selected from the group consisting of trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

Preferably, the C₃₋₈ cycloalkyl substituted with at least 3 hydroxyl groups is selected from the group consisting of cyclopentanetriol, cyclohexanetriol, cyclopentanetetrol, cyclohexanetetrol.

In one embodiment, the organic polyol is selected from the group consisting of monosaccharides, sugar alcohols, and sugar acids.

In one embodiment, the monosaccharide is selected from tetroses, pentoses, hexoses, heptoses. Preferably, the monosaccharide is selected from aldotetroses, ketotetroses, aldopentoses, ketopentoses, aldohexosen, ketohexoses, aldoheptoses, ketoheptoses.

In one embodiment, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.

In one embodiment, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.

In one embodiment, the sugar acid is selected from aldonic acids, ulosonic acids, uronic acids, aldaric acids. Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.

In one embodiment, the organic polyol is selected from the group consisting of glycerol, sorbitol, xylitol, mannitol, erythritol, maltitol, glucose, glucitol, ribulose, pentaerythritol, trimethylolpropane.

In one embodiment, the compound of formula 10 is selected from the group consisting of

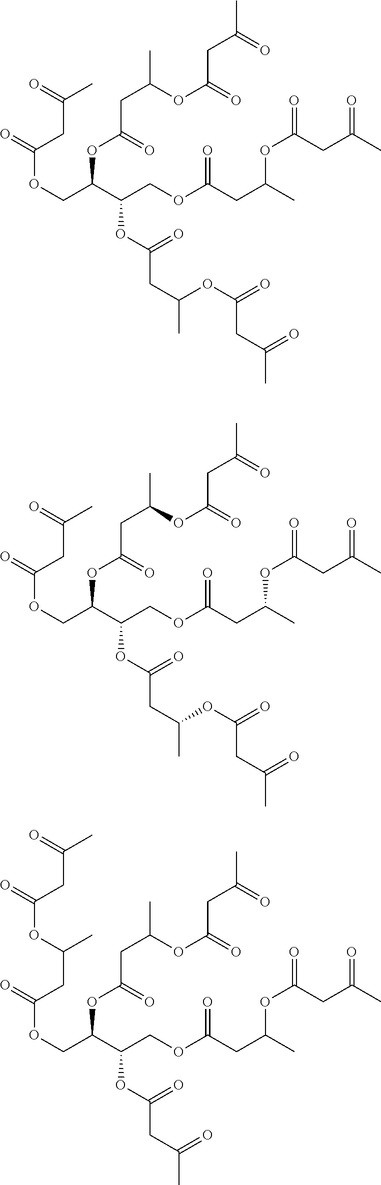 [SMILES: CC(=O)CC(=O)OC[C@@H](OC(=O)CC(C)OC(=O)CC(C)=O)[C@H](COC(=O)CC(C)OC(=O)CC(C)=O)OC(=O)CC(C)OC(=O)CC(C)=O ; CC(=O)CC(=O)OC[C@@H](OC(=O)C[C@@H](C)OC(=O)CC(C)=O)[C@H](COC(=O)C[C@@H](C)OC(=O)CC(C)=O)OC(=O)C[C@@H](C)OC(=O)CC(C)=O ; CC(=O)CC(=O)OC(C)CC(=O)OC[C@H](OC(=O)CC(C)=O)[C@@H](COC(=O)CC(C)OC(=O)CC(C)=O)OC(=O)CC(C)OC(=O)CC(C)=O]

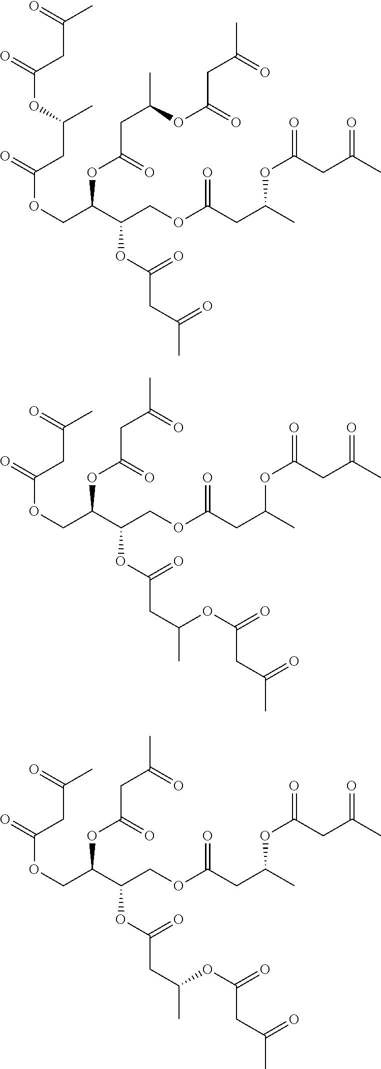

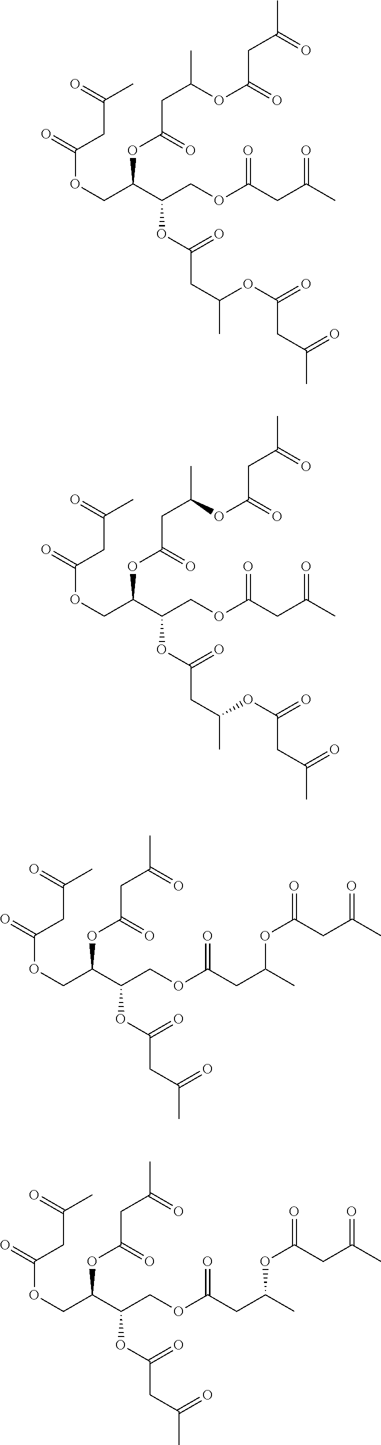

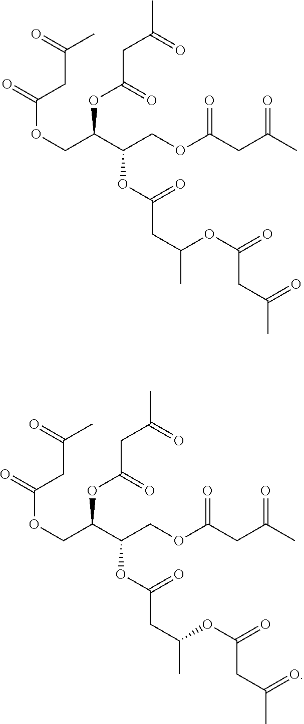 [SMILES: CC(=O)CC(=O)OC[C@H](OC(=O)CC(C)OC(=O)CC(C)=O)[C@@H](COC(=O)CC(C)=O)OC(=O)CC(C)=O ; CC(=O)CC(=O)OC[C@H](OC(=O)C[C@@H](C)OC(=O)CC(C)=O)[C@@H](COC(=O)CC(C)=O)OC(=O)CC(C)=O]

These exemplary compounds comprise AA residues and BHB residues in ratios of 2:1, 4:1 or 4:3.

In one embodiment, in the compound according to formula 10, all β-hydroxyl butyric acid ester units are either D-configured or L-configured. In another embodiment, all β-hydroxyl butyric acid ester units are present in the compound according to formula 10 as a non-racemic mixture of D- and L-configurations.

In one embodiment, the compound according to formula 10 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 10, all β-hydroxyl butyric acid ester units are either R-configured or S-configured. In another embodiment, all β-hydroxyl butyric acid ester units are present in the compound according to formula 10 as a non-racemic mixture of R- and S-configurations.

In one embodiment, the compound according to formula 10 contains more R-configured β-hydroxyl butyric acid ester units than S-configured β-hydroxyl butyric acid ester units. Preferably all β-hydroxyl butyric acid ester units are in R-configuration.

In another aspect, the present invention provides a process for the preparation of a compound of formula 10

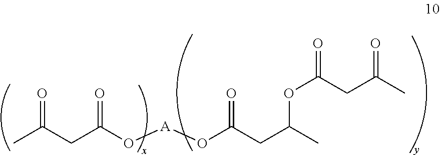

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) partially reacting an organic polyol of formula 2 with diketene 3 resulting in the formation of a compound according to formula 11;

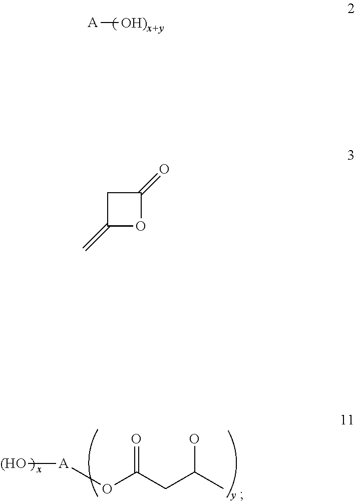

(ii) reacting the compound of formula 11 with hydrogen in the presence of a catalyst resulting in the formation of the compound according to formula 12

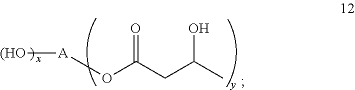

and
(iii) reacting the compound of formula 12 with diketene 3 resulting in the formation of the compound according to formula 10.

All embodiments and preferred embodiments disclosed above with respect to the compound of formula 10 likewise apply for the process of preparing a compound of formula 10.

The inventors surprisingly found that the process according to the present invention for the preparation of compounds according to formula 10 achieve significantly improved atom economy and cost efficiency per unit of acetoacetate if diketene 3 is employed directly in this reaction. Moreover, the inventive process for the preparation of compounds according to formula 10 provides an excellent process for providing polyalcohol esters comprising both BHB and AA units in one molecule.

In one embodiment, reaction step (i) is performed in the presence of an organic amine catalyst. In one embodiment, reaction step (iii) is performed in the presence of an organic amine catalyst.

Suitable organic amine catalysts include tertiary amines. Preferably, the organic amine catalyst is 1,4-diazabicyclo[2.2.2]octane (DABCO).

In step (ii) a compound of formula 11 is reacted with hydrogen in the presence of a catalyst to reduce an AA unit to a BHB unit. In one embodiment, reaction step (ii) is performed in the presence of a metal-based catalyst. Preferably, the metal-based catalyst is a Ni-based catalyst, a Pd-based catalyst, a Pt-based catalyst, a Ru-based catalyst, a Co-based catalyst, an Ir-based catalyst, or an Rh-based catalyst.

In one embodiment, reaction step (ii) is performed in presence of a chiral ligand capable of forming complexes with the metal-based catalyst. Preferred chiral ligand are selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino) butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C₃-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).

Preferably, reaction step (ii) is performed in the presence of a Ru-based catalyst. A preferred Ru-based catalyst is a Ruthenium oxide catalyst such as RuO₂. Further preferred Ru-based catalysts include Ru/C, RuAl₂O₃, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)-C3-TunePhos)(p-cymene)ruthenium(II)]chloride.

Depending on the type of the organic polyol, the process for the preparation of a compound of formula 10 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 10 is performed without a solvent. In another embodiment, the process for the preparation of a compound of formula 10 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, acetonitrile, toluene, chloroform, 1,4-dioxan,, or o/m/p-xylene. Preferably, the organic solvent is ethyl acetate.

In one embodiment, in the process for the preparation of a compound of formula 10, reaction step (i) is performed at temperature of 0-100° C. Preferably, reaction step (i) is performed at temperature of 15-70° C. Additionally, the reaction temperature of reaction step (i) may be maintained at 20-70° C. after complete addition of diketene 3.

In one embodiment, during reaction step (i) diketene 3 is slowly added over a period of 1-10 h, e.g. dropwise, to the reaction mixture, to avoid the formation of side products.

In one embodiment, in the process for the preparation of a compound of formula 10, reaction step (iii) is performed at temperature of 0-100° C. Preferably, reaction step (iii) is performed at temperature of 15-70° C. Additionally, the reaction temperature of reaction step (iii) may be maintained at 20-70° C. after complete addition of diketene 3.

In one embodiment, during reaction step (iii) diketene 3 is slowly added over a period of 1-10 h, e.g. dropwise, to the reaction mixture, to avoid the formation of side products.

In one embodiment reaction step (ii) is performed in a closed vessel under hydrogen pressure. Preferably, reaction step (ii) is performed at 5-30 bar hydrogen pressure and even more preferably at 10-20 bar hydrogen pressure.

In one embodiment, reaction step (ii) is performed at a temperature of 20-90° C. Preferably, reaction step (ii) is performed at a temperature of 50-70° C. and more preferably, reaction step (ii) is performed at a temperature of about 60° C.

In one embodiment, reaction step (ii) is stirred at 800-1200 rpm so as to ensure sufficient hydrogen diffusion into the reaction mixture.

An exemplary process for the preparation of compounds according to formula 10 is provided in the following reaction scheme:

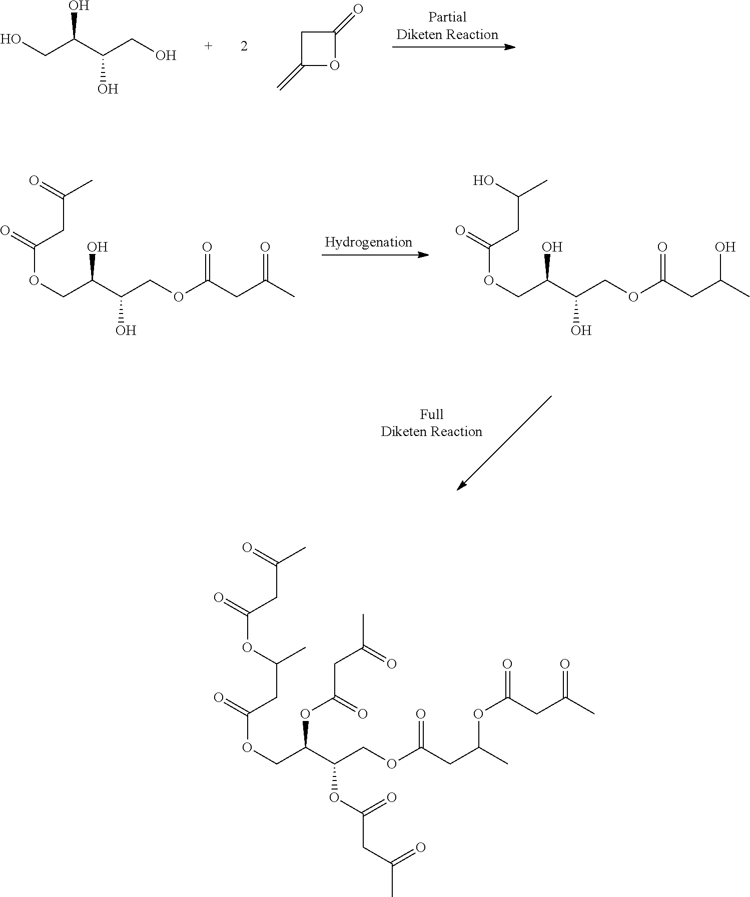

In another aspect, the present invention provides a compound of formula 13

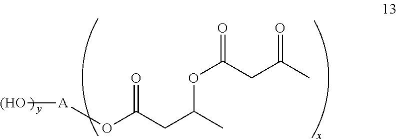

wherein
A is derived from an organic polyol with at least 3 hydroxyl groups,
x is at least 1,
y is at least 1, and
x+y is from 3 to the number of hydroxyl groups of the initial organic polyol A.

In one embodiment, the organic polyol has from 3 to 10 hydroxyl groups. Preferably, the organic polyol has from 3 to 8 hydroxyl groups, such as from 3 to 7 hydroxyl groups, from 3 to 6 hydroxyl groups, more preferably from 3 to 5 hydroxyl groups, or from 3 to 4 hydroxyl groups.

In one embodiment, x is at least 2, at least 3, or at least 4. In one embodiment, x is 1, 2, 3, 4, 5, or 6. Preferably, x is 1, 2, 3, or 4.

In one embodiment, y is at least 2, at least 3, or at least 4. In one embodiment, y is 1, 2, 3, 4, 5, or 6. Preferably, y is 1, 2, 3, or 4.

In one embodiment, x is 1 and y is 3. In one embodiment, x is 2 and y is 2. In one embodiment, x is 3 and y is 1.

In one embodiment, x+y is from 3 to 10, such as from 3 to 8, from 3 to 7, from 3 to 6, from 3 to 5 or from 3 to 4. Accordingly, x+y may be 3, 4, 5, 6, 7, 8, 9, or 10, preferably 3, 4, 5, or 6, more preferably, 3, 4, or 5.

In one embodiment, x+y is equal to the number of hydroxyl groups of the initial polyol A. In one embodiment, x+y is less than the number of hydroxyl groups of the initial polyol A.

In one embodiment, the organic polyol is a linear, branched, or cyclic organic compound with 2 to 18 carbon atoms having at least three hydroxyl groups.

In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

In another preferred embodiment, the linear or branched C₂₋₁₂ alkyl substituted with at least 3 hydroxyl groups is selected from the group consisting of trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

Preferably, the C₃₋₈ cycloalkyl substituted with at least 3 hydroxyl groups is selected from the group consisting of cyclopentanetriol, cyclohexanetriol, cyclopentanetetrol, cyclohexanetetrol.

In one embodiment, the organic polyol is selected from the group consisting of monosaccharides, sugar alcohols, and sugar acids.

In one embodiment, the monosaccharide is selected from tetroses, pentoses, hexoses, heptoses. Preferably, the monosaccharide is selected from aldotetroses, ketotetroses, aldopentoses, ketopentoses, aldohexosen, ketohexoses, aldoheptoses, ketoheptoses.

In one embodiment, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.

In one embodiment, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.

In one embodiment, the sugar acid is selected from aldonic acids, ulosonic acids, uronic acids, aldaric acids. Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.

In one embodiment, the organic polyol is selected from the group consisting of glycerol, sorbitol, xylitol, mannitol, erythritol, maltitol, glucose, glucitol, ribulose, pentaerythritol, trimethylolpropane.

In one embodiment, the compound of formula 13 is selected from the group consisting of

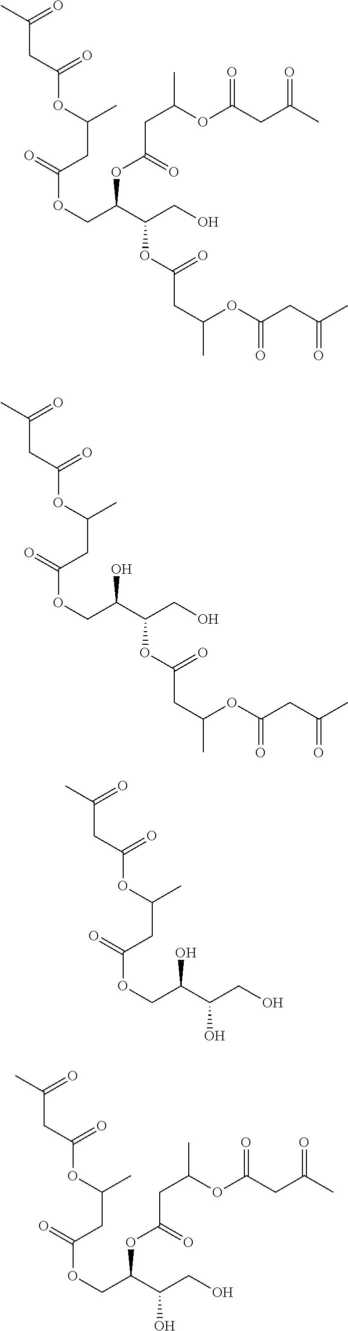

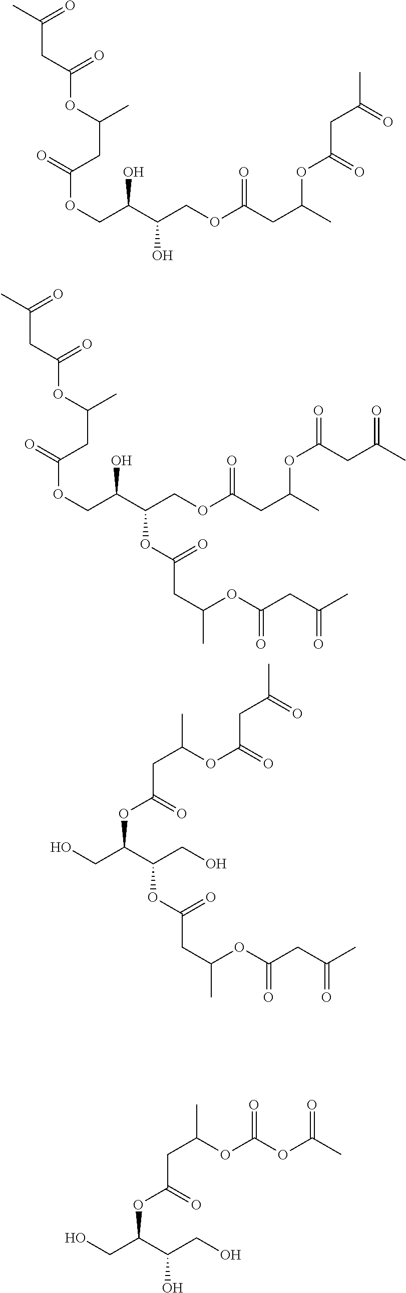

In one embodiment, in the compound according to formula 13, all β-hydroxyl butyric acid ester units are either D-configured or L-configured. In another embodiment, all β-hydroxyl butyric acid ester units are present in the compound according to formula 13 as a non-racemic mixture of D- and L-configurations.

In one embodiment, the compound according to formula 13 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 13, all β-hydroxyl butyric acid ester units are either R-configured or S-configured. In another embodiment, all β-hydroxyl butyric acid ester units are present in the compound according to formula 13 as a non-racemic mixture of R- and S-configurations.

In one embodiment, the compound according to formula 13 contains more R-configured β-hydroxyl butyric acid ester units than S-configured β-hydroxyl butyric acid ester units. Preferably all β-hydroxyl butyric acid ester units are in R-configuration.

In another aspect, the present invention provides a process for the preparation of a compound of formula 13

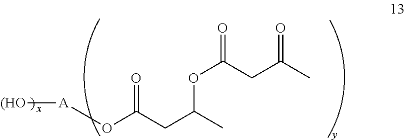

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) partially reacting an organic polyol of formula 2 with diketene 3 resulting in the formation of a compound according to formula 11;

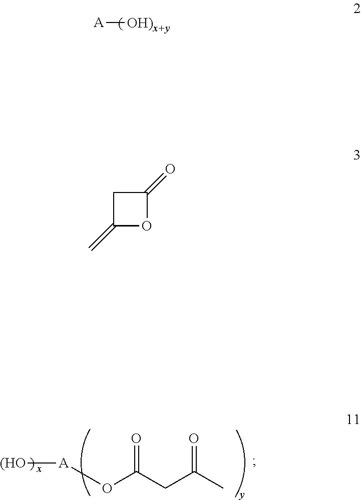

(ii) reacting the compound of formula 11 with hydrogen in the presence of a catalyst resulting in the formation of the compound according to formula 12

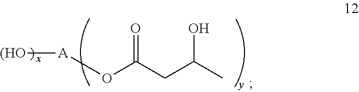

and
(iii) partially reacting the compound of formula 12 with diketene 3 resulting in the formation of the compound according to formula 13.

All embodiments and preferred embodiments disclosed above with respect to the compound of formula 13 likewise apply for the process of preparing a compound of formula 13.

In one embodiment, reaction step (i) is performed in the presence of an organic amine catalyst. In one embodiment, reaction step (iii) is performed in the presence of an organic amine catalyst.

Suitable organic amine catalysts include tertiary amines. Preferably, the organic amine catalyst is 1,4-diazabicyclo[2.2.2]octane (DABCO).

In step (ii) a compound of formula 11 is reacted with hydrogen in the presence of a catalyst to reduce an AA unit to a BHB unit. In one embodiment, reaction step (ii) is performed in the presence of a metal-based catalyst. Preferably, the metal-based catalyst is a Ni-based catalyst, a Pd-based catalyst, a Pt-based catalyst, a Ru-based catalyst, a Co-based catalyst, an Ir-based catalyst, or an Rh-based catalyst.

In one embodiment, reaction step (ii) is performed in presence of a chiral ligand capable of forming complexes with the metal-based catalyst. Preferred chiral ligand are selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino) butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).

Preferably, reaction step (ii) is performed in the presence of a Ru-based catalyst. A preferred Ru-based catalyst is a Ruthenium oxide catalyst such as RuO₂. Further preferred Ru-based catalysts include Ru/C, RuAl₂O₃, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)-C3-TunePhos)(p-cymene)ruthenium(II)]chloride.

Depending on the type of the organic polyol, the process for the preparation of a compound of formula 13 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 13 is performed without a solvent. In another embodiment, the process for the preparation of a compound of formula 13 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, acetonitrile, toluene, chloroform, 1,4-dioxan,, or o/m/p-xylene. Preferably, the organic solvent is ethyl acetate.

In one embodiment, in the process for the preparation of a compound of formula 13, reaction step (i) is performed at temperature of 0-100° C. Preferably, reaction step (i) is performed at temperature of 15-70° C. Additionally, the reaction temperature of reaction step (i) may be maintained at 20-70° C. after complete addition of diketene 3.

In one embodiment, during reaction step (i) diketene 3 is slowly added over a period of 1-10 h, e.g. dropwise, to the reaction mixture, to avoid the formation of side products.

In one embodiment, in the process for the preparation of a compound of formula 13, reaction step (iii) is performed at temperature of 0-100° C. Preferably, reaction step (iii) is performed at temperature of 15-70° C. Additionally, the reaction temperature of reaction step (iii) may be maintained at 20-70° C. after complete addition of diketene 3.

In one embodiment, during reaction step (iii) diketene 3 is slowly added over a period of 1-10 h, e.g. dropwise, to the reaction mixture, to avoid the formation of side products.

In one embodiment reaction step (ii) is performed in a closed vessel under hydrogen pressure. Preferably, reaction step (ii) is performed at 5-30 bar hydrogen pressure and even more preferably at 10-20 bar hydrogen pressure.

In one embodiment, reaction step (ii) is performed at a temperature of 30-90° C. Preferably, reaction step (ii) is performed at a temperature of 50-70° C. and more preferably, reaction step (ii) is performed at a temperature of about 60° C.

In one embodiment, reaction step (ii) is stirred at 800-1200 rpm so as to ensure sufficient hydrogen diffusion into the reaction mixture.

In another aspect, the present invention provides a compound of formula 14

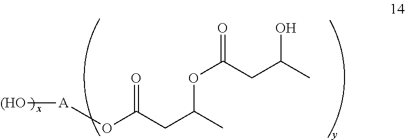

wherein
A is derived from an organic polyol with at least 3 hydroxyl groups,
x is at least 1,
y is at least 1, and
x+y is from 3 to the number of hydroxyl groups of the initial organic polyol A.

In one embodiment, the organic polyol has from 3 to 10 hydroxyl groups. Preferably, the organic polyol has from 3 to 8 hydroxyl groups, such as from 3 to 7 hydroxyl groups, from 3 to 6 hydroxyl groups, more preferably from 3 to 5 hydroxyl groups, or from 3 to 4 hydroxyl groups.

In one embodiment, x is at least 2, at least 3, or at least 4. In one embodiment, x is 1, 2, 3, 4, 5, or 6. Preferably, x is 1, 2, 3, or 4.

In one embodiment, y is at least 2, at least 3, or at least 4. In one embodiment, y is 1, 2, 3, 4, 5, or 6. Preferably, y is 1, 2, 3, or 4.

In one embodiment, x is 1 and y is 3. In one embodiment, x is 2 and y is 2. In one embodiment, x is 3 and y is 1.

In one embodiment, x+y is from 3 to 10, such as from 3 to 8, from 3 to 7, from 3 to 6, from 3 to 5 or from 3 to 4. Accordingly, x+y may be 3, 4, 5, 6, 7, 8, 9, or 10, preferably 3, 4, 5, or 6, more preferably 3, 4, or 5.

In one embodiment, x+y is equal to the number of hydroxyl groups of the initial polyol A. In one embodiment, x+y is less than the number of hydroxyl groups of the initial polyol A.

In one embodiment, the organic polyol is a linear, branched, or cyclic organic compound with 2 to 18 carbon atoms having at least three hydroxyl groups.

In one embodiment, the organic polyol is selected from a linear or branched C₂₋₁₂ alkyl substituted with at least 3 hydroxyl groups or a C₃₋₈ cycloalkyl substituted with at least 3 hydroxyl groups.

Preferably, the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

In another preferred embodiment, the linear or branched C₂₋₁₂ alkyl substituted with at least 3 hydroxyl groups is selected from the group consisting of trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

Preferably, the C₃₋₈ cycloalkyl substituted with at least 3 hydroxyl groups is selected from the group consisting of cyclopentanetriol, cyclohexanetriol, cyclopentanetetrol, cyclohexanetetrol.

In one embodiment, the organic polyol is selected from the group consisting of monosaccharides, sugar alcohols, and sugar acids.

In one embodiment, the monosaccharide is selected from tetroses, pentoses, hexoses, heptoses. Preferably, the monosaccharide is selected from aldotetroses, ketotetroses, aldopentoses, ketopentoses, aldohexosen, ketohexoses, aldoheptoses, ketoheptoses.

In one embodiment, the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.

In one embodiment, the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.

In one embodiment, the sugar acid is selected from aldonic acids, ulosonic acids, uronic acids, aldaric acids. Preferably, the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.

In one embodiment, the organic polyol is selected from the group consisting of glycerol, sorbitol, xylitol, mannitol, erythritol, maltitol, glucose, glucitol, ribulose, pentaerythritol, trimethylolpropane.

In one embodiment, the compound of formula 14 is selected from the group consisting of

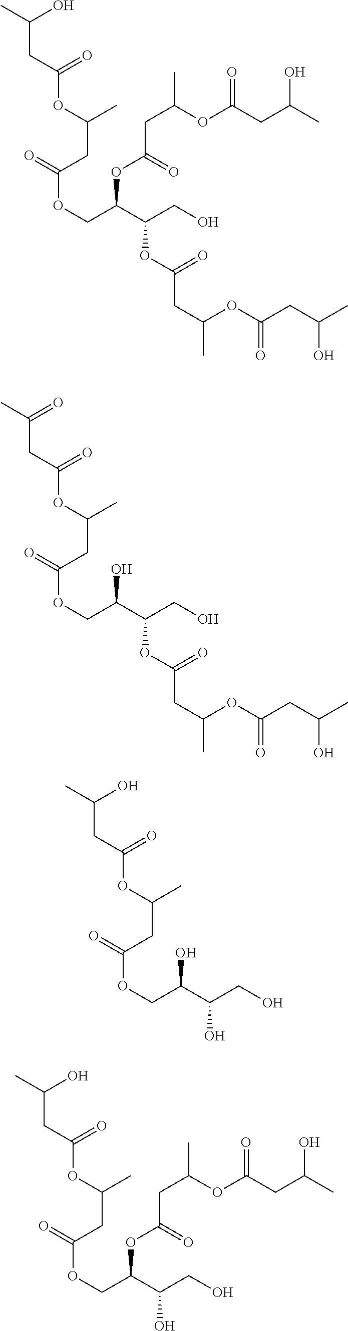

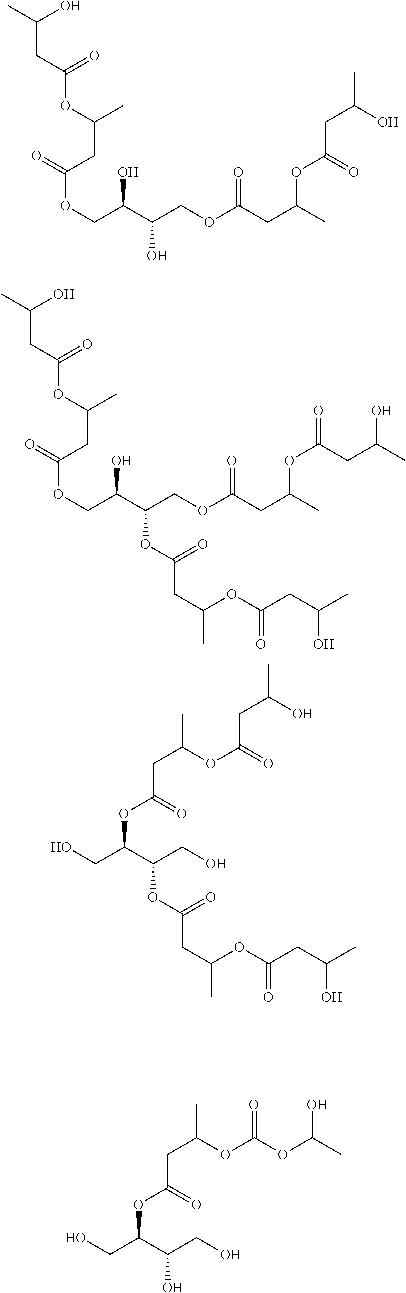

In one embodiment, in the compound according to formula 14, all β-hydroxyl butyric acid ester units are either D-configured or L-configured. In another embodiment, all β-hydroxyl butyric acid ester units are present in the compound according to formula 14 as a non-racemic mixture of D- and L-configurations.

In one embodiment, the compound according to formula 14 contains more D-configured β-hydroxyl butyric acid ester units than L-configured β-hydroxyl butyric acid ester units. Preferably all β-hydroxyl butyric acid ester units are in D-configuration.

In one embodiment, in the compound according to formula 14, all β-hydroxyl butyric acid ester units are either R-configured or S-configured. In another embodiment, all β-hydroxyl butyric acid ester units are present in the compound according to formula 14 as a non-racemic mixture of R- and S-configurations.

In one embodiment, the compound according to formula 14 contains more R-configured β-hydroxyl butyric acid ester units than S-configured β-hydroxyl butyric acid ester units. Preferably all β-hydroxyl butyric acid ester units are in R-configuration.

In another aspect, the present invention provides a process for the preparation of a compound of formula 14

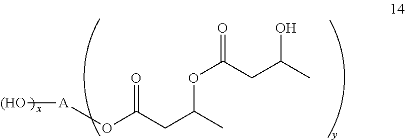

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) partially reacting an organic polyol of formula 2 with diketene 3 resulting in the formation of a compound according to formula 11;

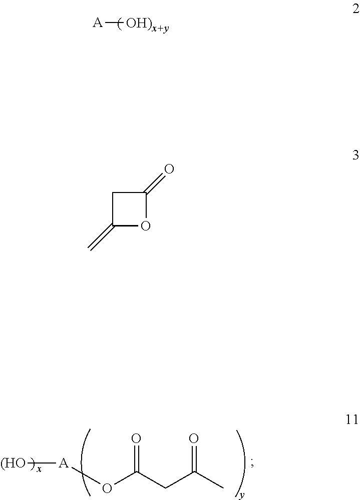

(ii) reacting the compound of formula 11 with hydrogen in the presence of a catalyst resulting in the formation of the compound according to formula 12

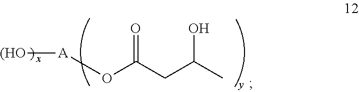

(iii) partially reacting the compound of formula 12 with diketene 3 resulting in the formation of the compound according to formula 13

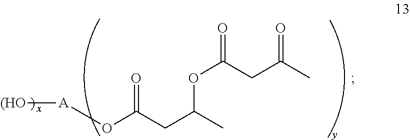

and
(iv) reacting the compound of formula 13 with hydrogen in the presence of a catalyst resulting in the formation of the compound according to formula 14.

All embodiments and preferred embodiments disclosed above with respect to the compound of formula 14 likewise apply for the process of preparing a compound of formula 14.

In one embodiment, reaction step (i) is performed in the presence of an organic amine catalyst. In one embodiment, reaction step (iii) is performed in the presence of an organic amine catalyst.

Suitable organic amine catalysts include tertiary amines. Preferably, the organic amine catalyst is 1,4-diazabicyclo[2.2.2]octane (DABCO).

In steps (ii) and (iv) a compound of formula 11 and a compound of formula 13, respectively, are reacted with hydrogen in the presence of a catalyst to reduce an AA unit to a BHB unit. In one embodiment, reaction step (ii) is performed in the presence of a metal-based catalyst. In one embodiment, reaction step (iv) is performed in the presence of a metal-based catalyst. In one embodiment, reaction steps (ii) and (iv) are performed in the presence of a metal-based catalyst. Preferably, the metal-based catalyst is a Ni-based catalyst, a Pd-based catalyst, a Pt-based catalyst, a Ru-based catalyst, a Co-based catalyst, an Ir-based catalyst, or an Rh-based catalyst.

In one embodiment, reaction step (ii) is performed in presence of a chiral ligand capable of forming complexes with the metal-based catalyst. In one embodiment, reaction step (iv) is performed in presence of a chiral ligand capable of forming complexes with the metal-based catalyst. In one embodiment, reaction steps (ii) and (iv) are performed in presence of a chiral ligand capable of forming complexes with the metal-based catalyst. Preferred chiral ligand are selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino) butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).

Preferably, reaction step (ii) is performed in the presence of a Ru-based catalyst. Preferably, reaction step (iv) is performed in the presence of a Ru-based catalyst. A preferred Ru-based catalyst is a Ruthenium oxide catalyst such as RuO₂. Further preferred Ru-based catalysts include Ru/C, RuAl₂O₃, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)-C3-TunePhos)(p-cymene)ruthenium(II)] chloride.

Depending on the type of the organic polyol, the process for the preparation of a compound of formula 14 may be performed in an organic solvent or without a solvent. Specifically, for liquid organic polyols or organic polyols having a low melting point (typically <120° C.), no organic solvent is necessary and the process can be performed without a solvent. Accordingly, in one embodiment, the process for the preparation of a compound of formula 14 is performed without a solvent. In another embodiment, the process for the preparation of a compound of formula 14 is performed in an organic solvent.

Suitable organic solvents include ethyl acetate, diethyl ether, MTBE, tetrahydrofurane, n-pentan, cyclopentan, n-Hexane, cyclohexane, n-heptan, DMF, DMSO, acetone, acetonitrile, toluene, chloroform, 1,4-dioxan,, or o/m/p-xylene. Preferably, the organic solvent is ethyl acetate.

In one embodiment, in the process for the preparation of a compound of formula 14, reaction step (i) is performed at temperature of 0-100° C. Preferably, reaction step (i) is performed at temperature of 15-70° C. Additionally, the reaction temperature of reaction step (i) may be maintained at 20-70° C. after complete addition of diketene 3.

In one embodiment, during reaction step (i) diketene 3 is slowly added over a period of 1-10 h, e.g. dropwise, to the reaction mixture, to avoid the formation of side products.

In one embodiment, in the process for the preparation of a compound of formula 14, reaction step (iii) is performed at temperature of 20-100° C. Preferably, reaction step (iii) is performed at temperature of 40-70° C. Additionally, the reaction temperature of reaction step (iii) may be maintained at 40-70° C. after complete addition of diketene 3.

In one embodiment, during reaction step (iii) diketene 3 is slowly added over a period of 1-6 h, e.g. dropwise, to the reaction mixture, to avoid the formation of side products.

In one embodiment reaction step (ii) is performed in a closed vessel under hydrogen pressure. In one embodiment reaction step (iv) is performed in a closed vessel under hydrogen pressure. In one embodiment reaction steps (ii) and (iv) are performed in a closed vessel under hydrogen pressure. Preferably, reaction step (ii) is performed at 5-30 bar hydrogen pressure and even more preferably at 10-20 bar hydrogen pressure. Preferably, reaction step (iv) is performed at 5-30 bar hydrogen pressure and even more preferably at 10-20 bar hydrogen pressure.

In one embodiment, reaction step (ii) is performed at a temperature of 30-90° C. Preferably, reaction step (ii) is performed at a temperature of 50-70° C. and more preferably, reaction step (ii) is performed at a temperature of about 60° C. In one embodiment, reaction step (iv) is performed at a temperature of 30-90° C. Preferably, reaction step (iv) is performed at a temperature of 50-70° C. and more preferably, reaction step (iv) is performed at a temperature of about 60° C.

In one embodiment, reaction step (ii) is stirred at 800-1200 rpm so as to ensure sufficient hydrogen diffusion into the reaction mixture. In one embodiment, reaction step (iv) is stirred at 800-1200 rpm so as to ensure sufficient hydrogen diffusion into the reaction mixture.

Preferred embodiments of the present invention are further defined in the following numbered items:
1. A compound of formula 1

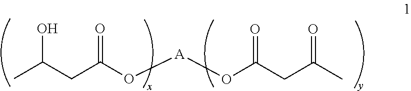

wherein
A is derived from an organic polyol with at least 3 hydroxyl groups,
x is at least 1,
y is at least 1, and
x+y is from 3 to the number of hydroxyl groups of the initial organic polyol A.
2. The compound according to item 1, wherein the organic polyol is selected from a linear or branched C₂₋₁₂ alkyl substituted with at least 3 hydroxyl groups, a C₃₋₈ cycloalkyl substituted with at least 3 hydroxyl groups.
3. The compound according to item 2, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.
4. The compound according to item 2 or 3, wherein the C₃₋₈ cycloalkyl substituted with at least 3 hydroxyl groups is selected from the group consisting of cyclopentanetriol, cyclohexanetriol, cyclopentanetetrol, cyclohexanetetrol.
5. The compound according to item 1, wherein the organic polyol is selected from the group consisting of monosaccharides, sugar alcohols, sugar acids.
6. The compound according to item 5, wherein the monosaccharide is selected from tetroses, pentoses, hexoses, heptoses, preferably wherein the monosaccharide is selected from aldotetroses, ketotetroses, aldopentoses, ketopentoses, aldohexosen, ketohexoses, aldoheptoses, ketoheptoses.
7. The compound according to item 5 or 6, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.
8. The compound according to any one of items 5 to 7, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.
9. The compound according to any one of items 5 to 8, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.
10. The compound according to any one of items 1 to 9, wherein the organic polyol is selected from the group consisting of glycerol, sorbitol, xylitol, mannitol, erythritol, maltitol, glucose, glucitol, ribulose, pentaerythritol, trimethylolpropane.
11. The compound according to any one of items 1 to 10, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.
12. The compound according to any one of items 1 to 11, wherein x+y is equal to the number of hydroxyl groups of the initial polyol A.
13. The compound according to any one of items 1 to 12, wherein the ratio of x to y is not 1:1 (x+y).
14. The compound according to item 13, wherein x>y, preferably wherein x:y is 2:1, 3:1, 4:1, 5:1, 6:1 or 7:1, more preferably wherein x:y is 2:1 or 3:1.
15. The compound according to item 13, wherein x<y, preferably wherein x:y is 1:2, 1:3, 1:4, 1:5, 1:6 or 1:7, more preferably wherein x:y is 1:2 or 1:3.
16. The compound according to any one of items 1 to 12, wherein the compound is selected from the group consisting of

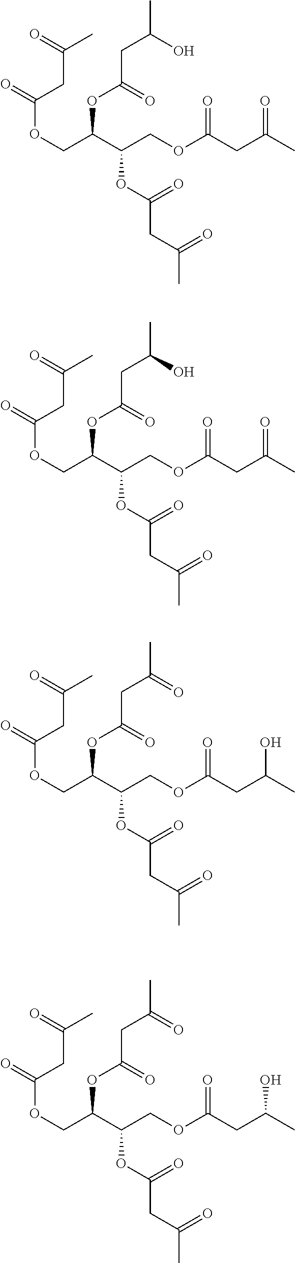 [SMILES: CC(=O)CC(=O)OC[C@H](OC(=O)CC(C)=O)[C@@H](COC(=O)CC(C)=O)OC(=O)CC(C)O ; CC(=O)CC(=O)OC[C@H](OC(=O)CC(C)=O)[C@@H](COC(=O)CC(C)=O)OC(=O)C[C@@H](C)O ; CC(=O)CC(=O)OC[C@@H](OC(=O)CC(C)=O)[C@H](COC(=O)CC(C)O)OC(=O)CC(C)=O ; CC(=O)CC(=O)OC[C@@H](OC(=O)CC(C)=O)[C@H](COC(=O)C[C@@H](C)O)OC(=O)CC(C)=O]

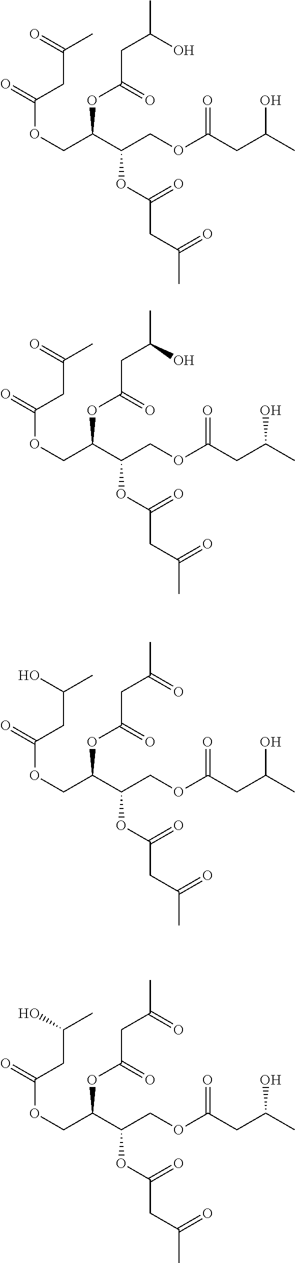

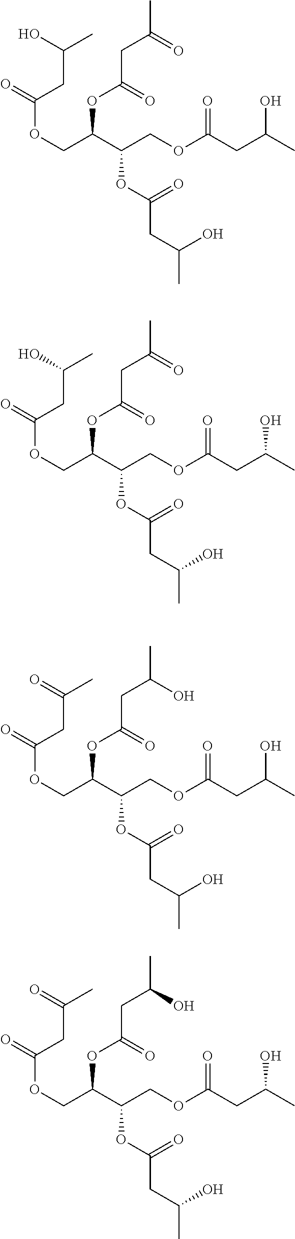

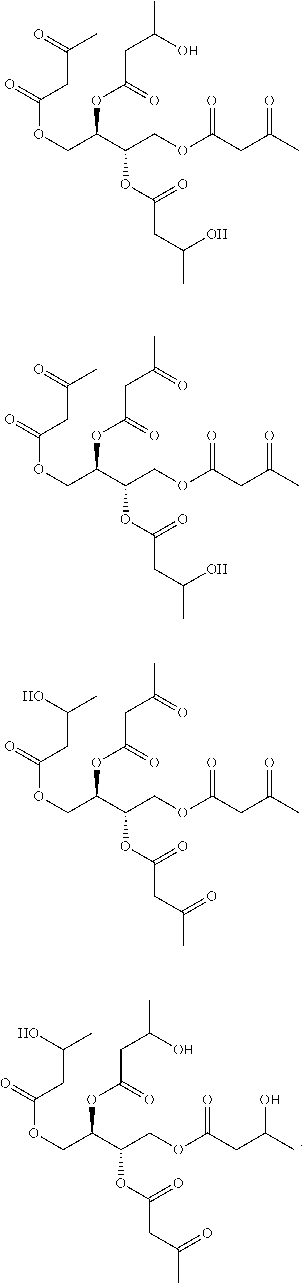 [SMILES: CC(=O)CC(=O)OC[C@H](OC(=O)CC(C)O)[C@@H](COC(=O)CC(C)=O)OC(=O)CC(C)O ; CC(=O)CC(=O)OC[C@H](OC(=O)CC(C)O)[C@@H](COC(=O)CC(C)=O)OC(=O)CC(C)=O ; CC(=O)CC(=O)OC[C@H](OC(=O)CC(C)=O)[C@@H](COC(=O)CC(C)O)OC(=O)CC(C)=O ; CC(=O)CC(=O)O[C@@H](COC(=O)CC(C)O)[C@@H](COC(=O)CC(C)O)OC(=O)CC(C)O]

17. The compound according to any one of items 1 to 13, wherein the compound is selected from the group consisting of

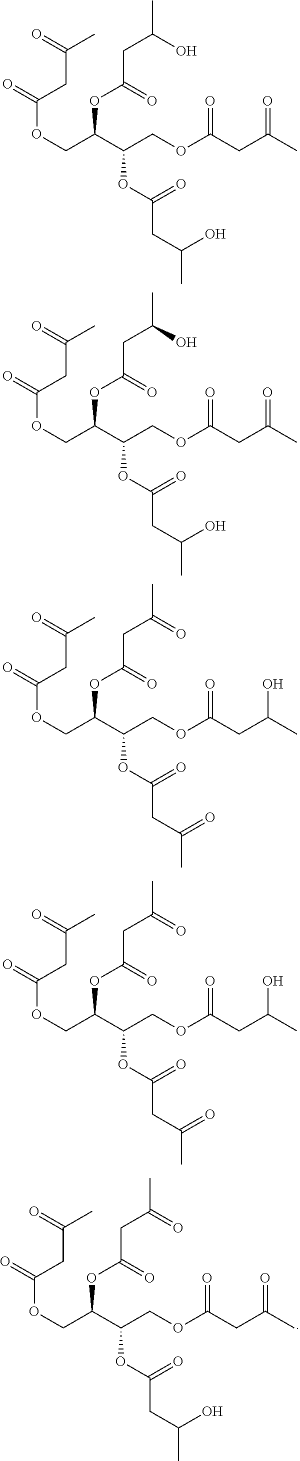

18. The compound according to any one of items 1 to 13, wherein the compound is selected from the group consisting of

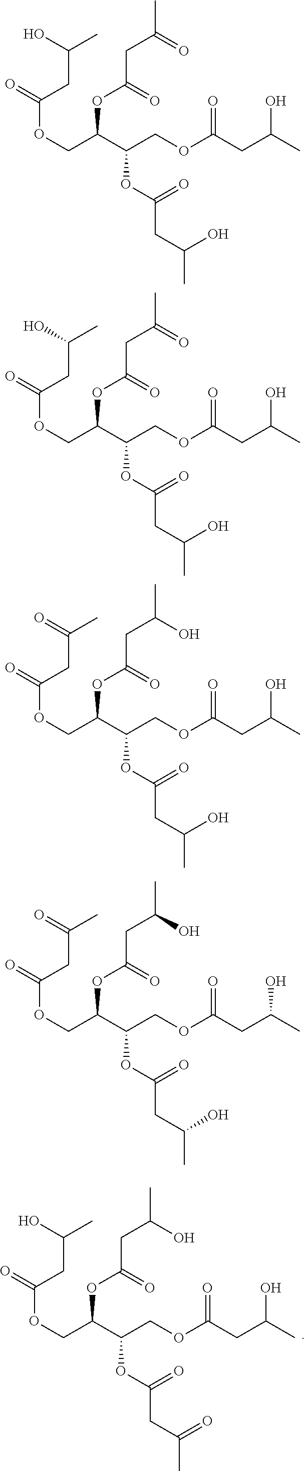

19. The compound according to any one of items 1 to 18, wherein all β-hydroxyl butyric acid ester units are either R-configured or S-configured, or all β-hydroxyl butyric acid ester units are present as a non-racemic mixture of R- and S-configurations.
20. The compound according to any one of items 1 to 19, wherein the compound contains more R-configured β-hydroxyl butyric acid ester units than S-configured β-hydroxyl butyric acid ester units, preferably wherein all β-hydroxyl butyric acid ester units are in R-configuration.
21. A process for the preparation of a compound of formula 1

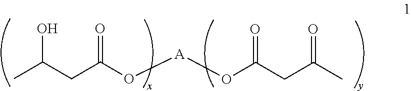

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) reacting an organic polyol of formula 2 with diketene 3 resulting in the formation of a compound according to formula 4;
and

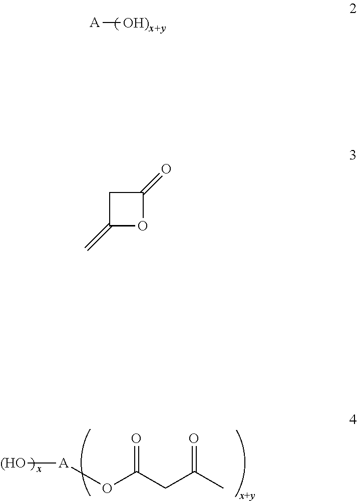

(ii) partially hydrogenating the compound of formula 4 by reacting the compound of formula 4 with hydrogen in the presence of a catalyst resulting in the formation of the compound according to formula 1.
22. A process for the preparation of a compound of formula 1

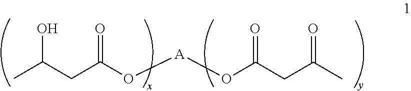

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) partially reacting an organic polyol of formula 2 with diketene 3 resulting in the formation of a compound according to formula 5;

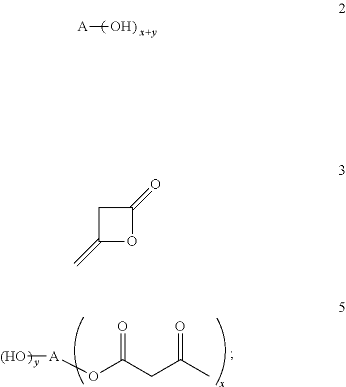

(ii) reacting the compound of formula 5 with hydrogen in the presence of a catalyst resulting in the formation of the compound according to formula 6

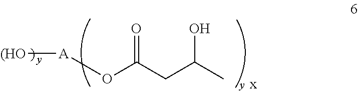

and
(iii) partially reacting the compound of formula 6 with diketene 3 resulting in the formation of the compound according to formula 1.
23. The process according to item 21 or 22, wherein the organic polyol is selected from a linear or branched C₂₋₁₂ alkyl substituted with at least 3 hydroxyl groups, a C₃₋₈ cycloalkyl substituted with at least 3 hydroxyl groups.
24. The process according to item 23, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.
25. The process according to item 23 or 24, wherein the C₃₋₈ cycloalkyl substituted with at least 3 hydroxyl groups is selected from the group consisting of cyclopentanetriol, cyclohexanetriol, cyclopentanetetrol, cyclohexanetetrol.
26. The process according to item 21 or 22, wherein the organic polyol is selected from the group consisting of monosaccharides, sugar alcohols, sugar acids.
27. The process according to item 26, wherein the monosaccharide is selected from tetroses, pentoses, hexoses, heptoses, preferably wherein the monosaccharide is selected from aldotetroses, ketotetroses, aldopentoses, ketopentoses, aldohexosen, ketohexoses, aldoheptoses, ketoheptoses.
28. The process according to item 26 or 27, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.
29. The process according to any one of items 26 to 28, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.
30. The process according to any one of items 26 to 29, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.
31. The process according to any one of items 21 to 30, wherein the organic polyol is selected from the group consisting of glycerol, sorbitol, xylitol, mannitol, erythritol, maltitol, glucose, glucitol, ribulose, pentaerythritol, trimethylolpropane.
32. The process according to any one of items 21 to 31, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.
33. The process according to any one of items 21 to 32, wherein x+y is equal to the number of hydroxyl groups of the initial polyol A.
34. The process according to any one of items 21 to 33, wherein reaction step (i) is performed in the presence of an organic amine catalyst.
35. The process according to any one of items 22 to 34, wherein reaction step (iii) is performed in the presence of an organic amine catalyst.
36. The process according to item 34 or 35, wherein the organic amine catalyst is a tertiary amine.
37. The process according to item 36, wherein the organic amine catalyst is DABCO.
38. The process according to any one of items 21 to 37, wherein reaction step (ii) is performed in the presence of a metal-based catalyst, preferably a Ni-based catalyst, a Pd-based catalyst, a Pt-based catalyst, a Ru-based catalyst, a Co-based catalyst, an Ir-based catalyst, or a Rh-based catalyst.
39. The process according to any one of items 21 to 38, wherein reaction step (ii) is performed in the presence of a Ru-based catalyst, preferably selected from a Ruthenium oxide catalyst, Ru/C, RuAl₂O₃, RuO₂, Ru(OAc)₂(BINAP), Ru(Cl)₂(BINAP), C3-[(S,S)-teth-MtsDpenRuCI], [(R)-BinapRuCl(p-cymene)]Cl, and [Chloro(R)-C3-TunePhos)(p-cymene)ruthenium(II)] chloride.
40. The process according to item 38 or 39, wherein reaction step (ii) is performed in the presence of a chiral ligand capable of forming complexes with the metal-based catalyst, preferably wherein the chiral ligand is selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino) butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).
41. The process according to any one of items 21 to 40, wherein all β-hydroxyl butyric acid ester units are either R-configured or S-configured, or all β-hydroxyl butyric acid ester units are present as a non-racemic mixture of R- and S-configurations.
42. The process according to any one of items 21 to 41, wherein the compound contains more R-configured β-hydroxyl butyric acid ester units than S-configured β-hydroxyl butyric acid ester units, preferably wherein all β-hydroxyl butyric acid ester units are in R-configuration.
43. The process according to any one of items 21 to 42, wherein the compound of formula 1 is selected from the group consisting of

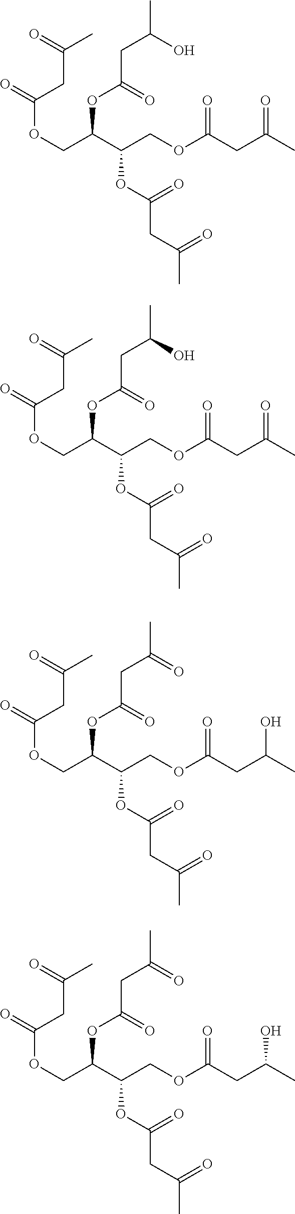

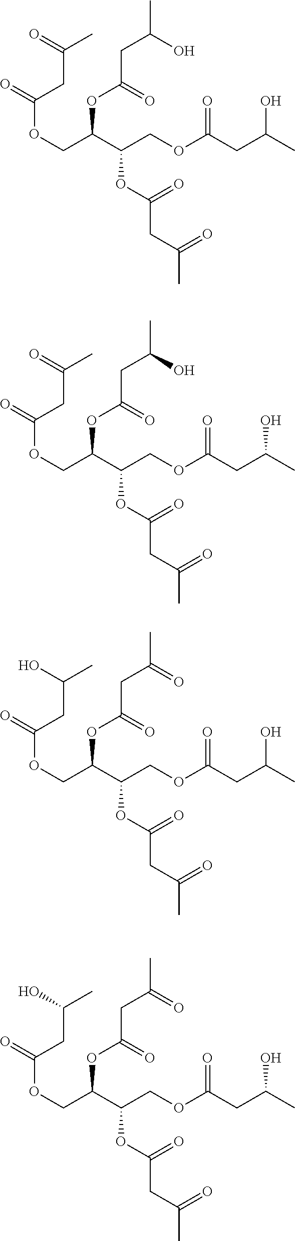

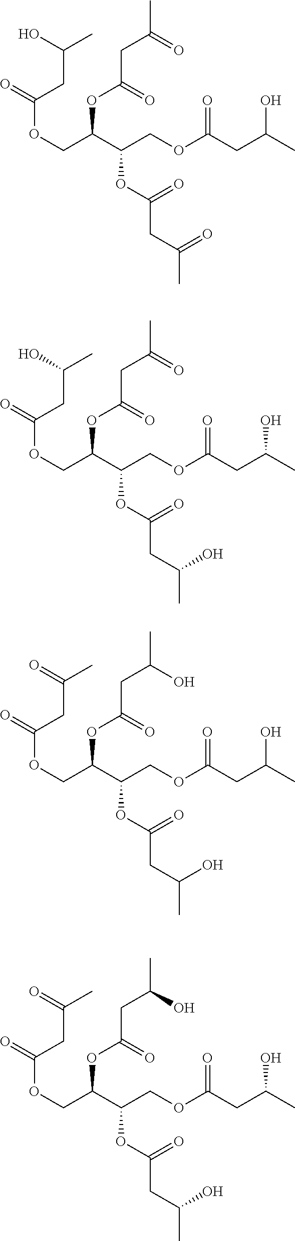

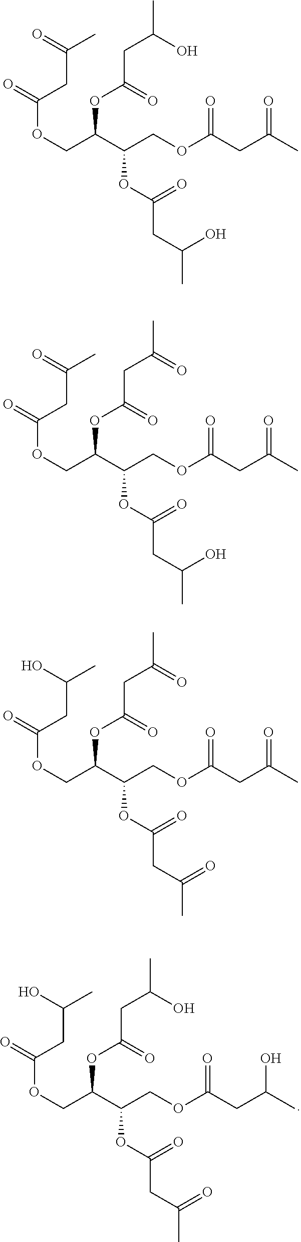

44. The process according to any one of items 21 to 43, wherein the ratio of x to y is not 1:1 (x+y).
45. The process according to item 44, wherein x>y, preferably wherein the ratio of x to y is between 7:1 and 2:1.
46. The process according to item 44, wherein x<y, preferably wherein the ratio of x to y is between 1:7 and 1:2.
47. The process according to any one of items 21 to 43, wherein the compound of formula 1 is selected from the group consisting of

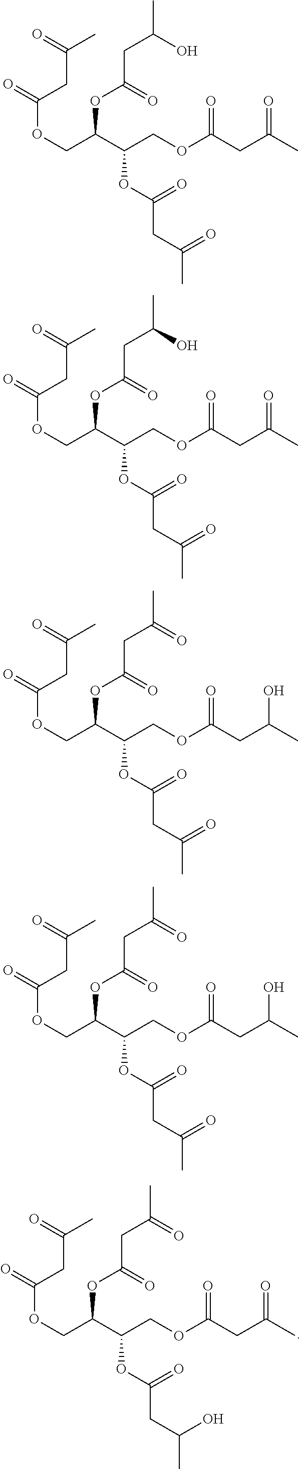

48. The process according to any one of items 21 to 43, wherein the compound of formula 1 is selected from the group consisting of

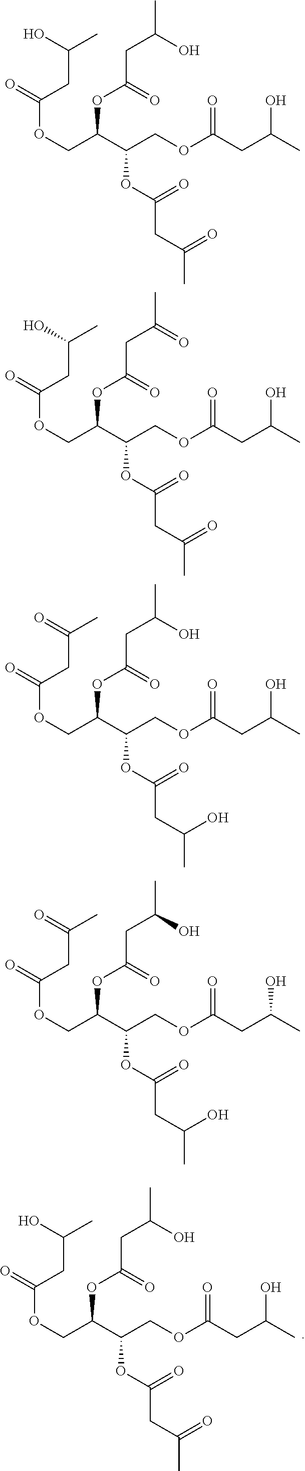

49. A compound of formula 7

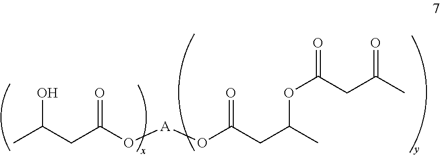

wherein
A is derived from an organic polyol with at least 3 hydroxyl groups,
x is at least 1,
y is at least 1, and
x+y is from 3 to the number of hydroxyl groups of the initial organic polyol A.
50. The compound according to item 49, wherein the organic polyol is selected from a linear or branched C₂₋₁₂ alkyl substituted with at least 3 hydroxyl groups, a C₃₋₈ cycloalkyl substituted with at least 3 hydroxyl groups.
51. The compound according to item 50, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.
52. The compound according to item 50 or 51, wherein the C₃₋₈ cycloalkyl substituted with at least 3 hydroxyl groups is selected from the group consisting of cyclopentanetriol, cyclohexanetriol, cyclopentanetetrol, cyclohexanetetrol.
53. The compound according to item 49, wherein the organic polyol is selected from the group consisting of monosaccharides, sugar alcohols, sugar acids.
54. The compound according to item 53, wherein the monosaccharide is selected from tetroses, pentoses, hexoses, heptoses, preferably wherein the monosaccharide is selected from aldotetroses, ketotetroses, aldopentoses, ketopentoses, aldohexosen, ketohexoses, aldoheptoses, ketoheptoses.
55. The compound according to item 53 or 54, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.
56. The compound according to any one of items 53 to 55, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.
57. The compound according to any one of items 53 to 56, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.
58. The compound according to any one of items 49 to 57, wherein the organic polyol is selected from the group consisting of glycerol, sorbitol, xylitol, mannitol, erythritol, maltitol, glucose, glucitol, ribulose, pentaerythritol, trimethylolpropane.
59. The compound according to any one of items 49 to 58, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.
60. The compound according to any one of items 49 to 59, wherein x+y is equal to the number of hydroxyl groups of the initial polyol A.
61. The compound according to any one of items 39 to 50, wherein the compound is selected from the group consisting of

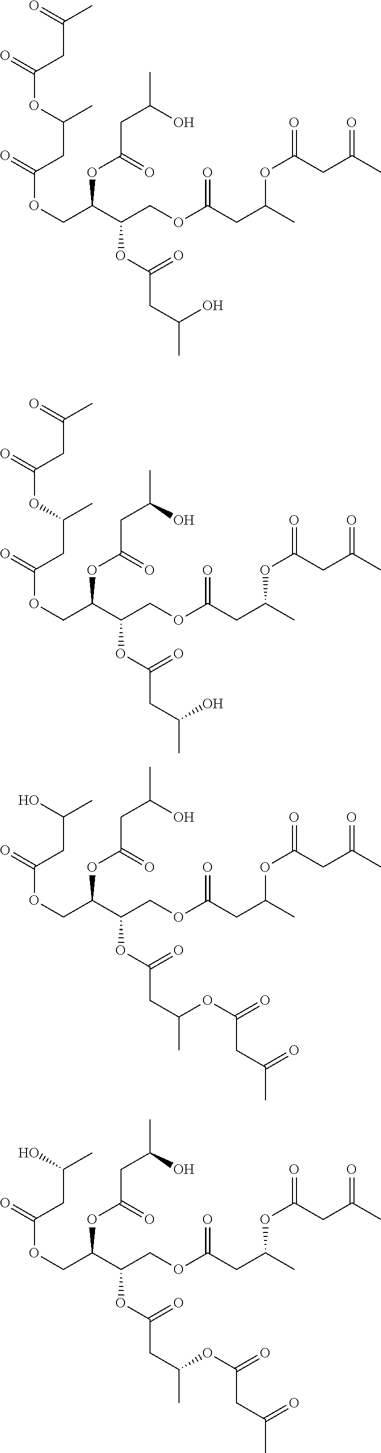

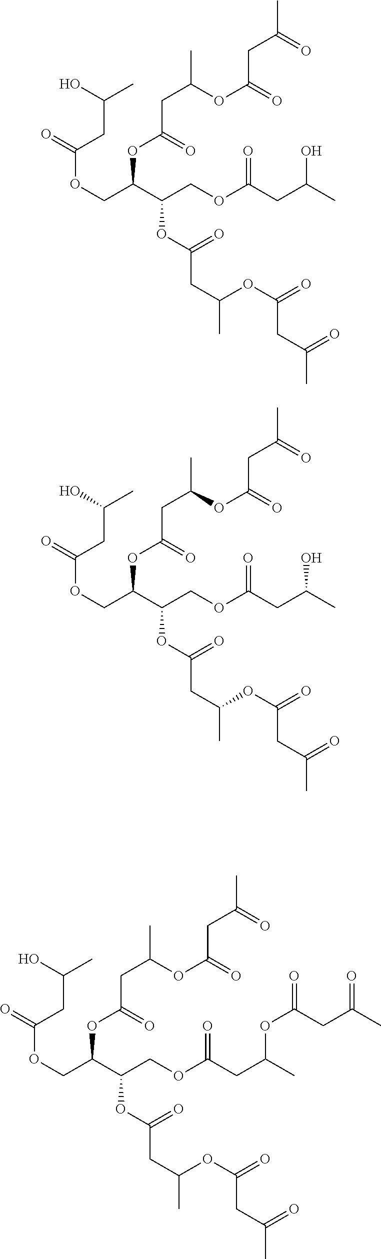

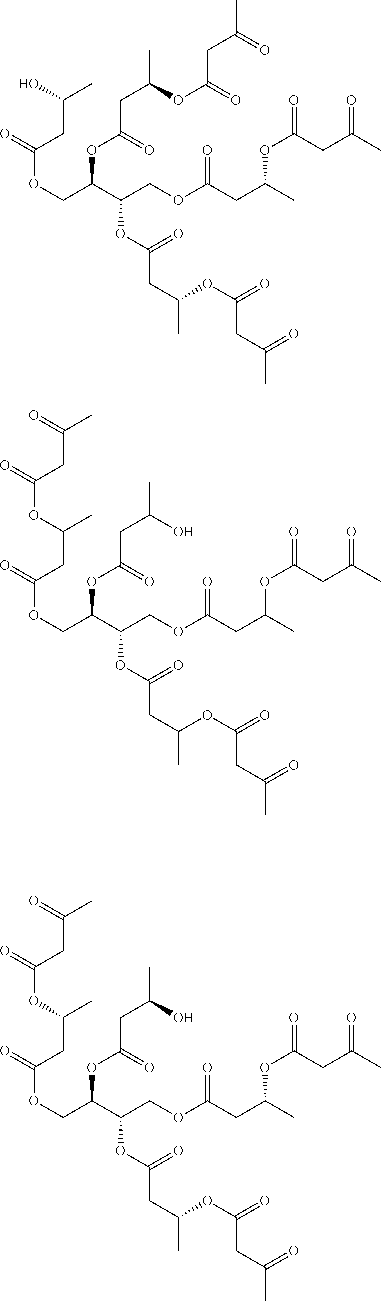 [SMILES: CC(=O)CC(=O)O[C@H](C)CC(=O)OC[C@H](OC(=O)C[C@@H](C)OC(=O)CC(C)=O)[C@@H](COC(=O)C[C@@H](C)O)OC(=O)C[C@@H](C)OC(=O)CC(C)=O ; CC(=O)CC(=O)OC(C)CC(=O)OC[C@H](OC(=O)CC(C)OC(=O)CC(C)=O)[C@@H](COC(=O)CC(C)OC(=O)CC(C)=O)OC(=O)CC(C)O ; CC(=O)CC(=O)O[C@H](C)CC(=O)OC[C@H](OC(=O)C[C@@H](C)OC(=O)CC(C)=O)[C@@H](COC(=O)C[C@@H](C)OC(=O)CC(C)=O)OC(=O)C[C@@H](C)O]

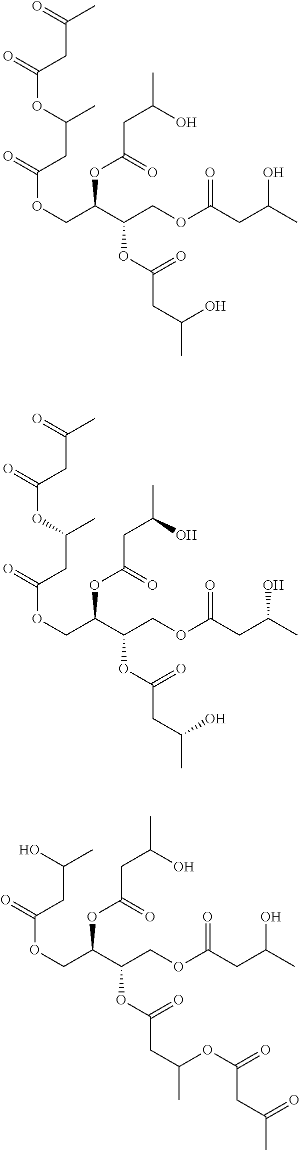 [SMILES: CC(=O)CC(=O)OC(C)CC(=O)OC[C@@H](OC(=O)CC(C)O)[C@H](COC(=O)CC(C)O)OC(=O)CC(C)O ; CC(=O)CC(=O)O[C@H](C)CC(=O)OC[C@@H](OC(=O)C[C@@H](C)O)[C@H](COC(=O)C[C@@H](C)O)OC(=O)C[C@@H](C)O ; CC(=O)CC(=O)OC(C)CC(=O)O[C@@H](COC(=O)CC(C)O)[C@@H](COC(=O)CC(C)O)OC(=O)CC(C)O]

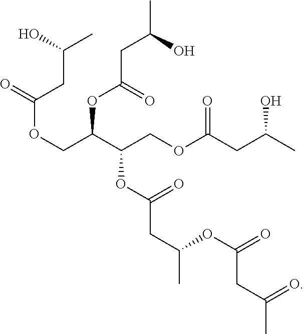 [SMILES: CC(=O)CC(=O)O[C@H](C)CC(=O)O[C@@H](COC(=O)C[C@@H](C)O)[C@@H](COC(=O)C[C@@H](C)O)OC(=O)C[C@@H](C)O]

62. The compound according to any one of items 49 to 61, wherein all β-hydroxyl butyric acid ester units are either R-configured or S-configured, or all β-hydroxyl butyric acid ester units are present as a non-racemic mixture of R- and S-configurations.
63. The compound according to any one of items 49 to 62, wherein the compound contains more R-configured β-hydroxyl butyric acid ester units than S-configured β-hydroxyl butyric acid ester units, preferably wherein all β-hydroxyl butyric acid ester units are in R-configuration.
64. A process for the preparation of a compound of formula 7

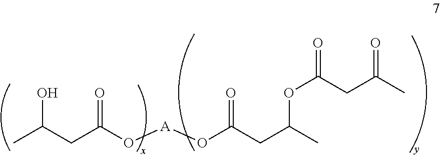

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) reacting an organic polyol of formula 2 with diketene 3 resulting in the formation of a compound according to formula 4;

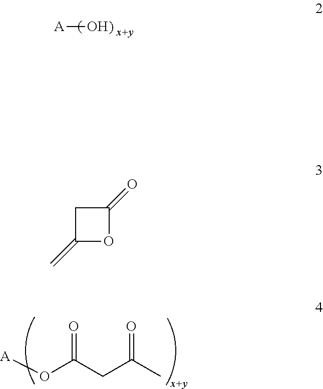

(ii) reacting the compound of formula 4 with hydrogen in the presence of a catalyst resulting in the formation of a compound according to formula 8;

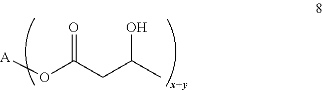

(iii) partially reacting the compound according to formula 8 with diketene 3 resulting in the formation of a compound according to formula 7.
65. The process according to item 64, wherein the organic polyol is selected from a linear or branched C₂₋₁₂ alkyl substituted with at least 3 hydroxyl groups, a C₃₋₈ cycloalkyl substituted with at least 3 hydroxyl groups.
66. The process according to item 65, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.
67. The process according to item 65 or 66, wherein the C₃₋₈ cycloalkyl substituted with at least 3 hydroxyl groups is selected from the group consisting of cyclopentanetriol, cyclohexanetriol, cyclopentanetetrol, cyclohexanetetrol.
68. The process according to item 64, wherein the organic polyol is selected from the group consisting of monosaccharides, sugar alcohols, sugar acids.
69. The process according to item 68, wherein the monosaccharide is selected from tetroses, pentoses, hexoses, heptoses, preferably wherein the monosaccharide is selected from aldotetroses, ketotetroses, aldopentoses, ketopentoses, aldohexosen, ketohexoses, aldoheptoses, ketoheptoses.
70. The process according to item 68 or 69, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.
71. The process according to any one of items 68 to 70, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.
72. The process according to any one of items 68 to 71, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.
73. The process according to any one of items 64 to 72, wherein the organic polyol is selected from the group consisting of glycerol, sorbitol, xylitol, mannitol, erythritol, maltitol, glucose, glucitol, ribulose, pentaerythritol, trimethylolpropane.
74. The process according to any one of items 64 to 73, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.
75. The process according to any one of items 64 to 74, wherein x+y is equal to the number of hydroxyl groups of the initial polyol A.
76. The process according to any one of items 64 to 75, wherein reaction step (i) is performed in the presence of an organic amine catalyst.
77. The process according to any one of items 64 to 76, wherein reaction step (iii) is performed in the presence of an organic amine catalyst.
78. The process according to item 76 or 77, wherein the organic amine catalyst is a tertiary amine.
79. The process according to item 78, wherein the organic amine catalyst is DABCO.
80. The process according to any one of items 64 to 79, wherein reaction step (ii) is performed in the presence of a metal-based catalyst, preferably a Ni-based catalyst, a Pd-based catalyst, a Pt-based catalyst, a Ru-based catalyst, a Co-based catalyst, an Ir-based catalyst, or a Rh-based catalyst.
81. The process according to any one of items 64 to 80, wherein reaction step (ii) is performed in the presence of a Ru-based catalyst, preferably selected from a Ruthenium oxide catalyst, Ru/C, RuAl₂O₃, RuO₂, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)-C3-TunePhos)(p-cymene)ruthenium(II)] chloride.
82. The process according to item 80 or 81, wherein reaction step (ii) is performed in the presence of a chiral ligand capable of forming complexes with the metal-based catalyst, preferably wherein the chiral ligand is selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino) butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).
83. The process according to any one of items 64 to 82, wherein all β-hydroxyl butyric acid ester units are either R-configured or S-configured, or all β-hydroxyl butyric acid ester units are present as a non-racemic mixture of R- and S-configurations.
84. The process according to any one of items 64 to 83, wherein the compound contains more R-configured β-hydroxyl butyric acid ester units than S-configured β-hydroxyl butyric acid ester units, preferably wherein all β-hydroxyl butyric acid ester units are in R-configuration.
85. The process according to any one of items 64 to 84, wherein the compound is selected from the group consisting of

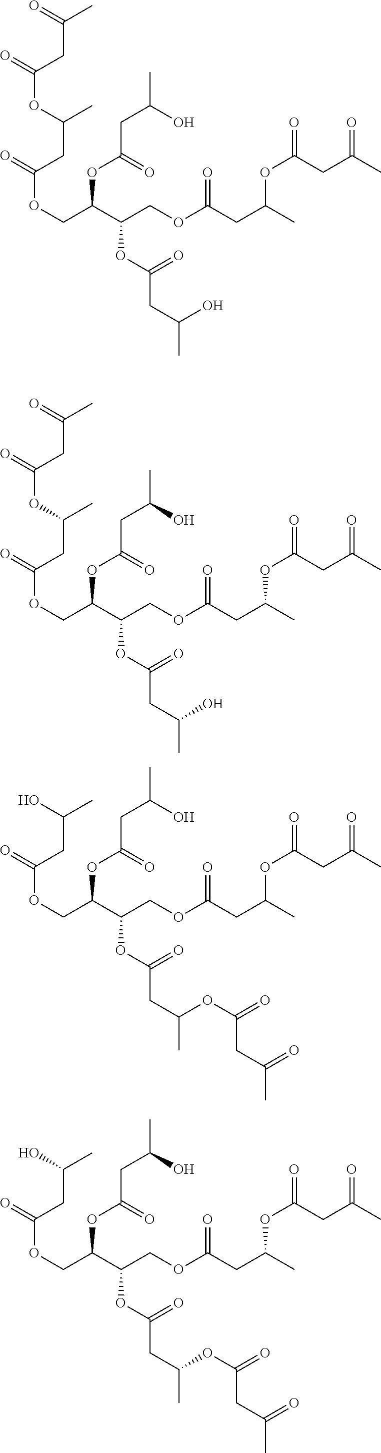

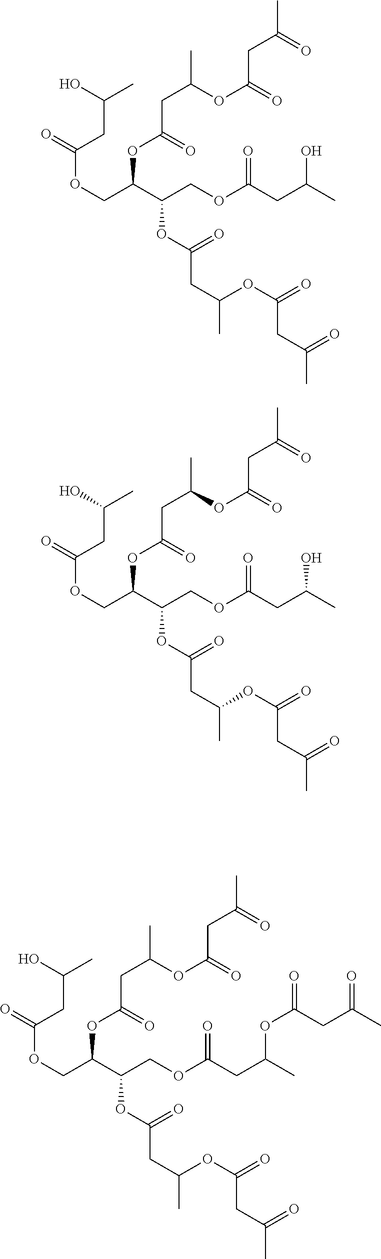

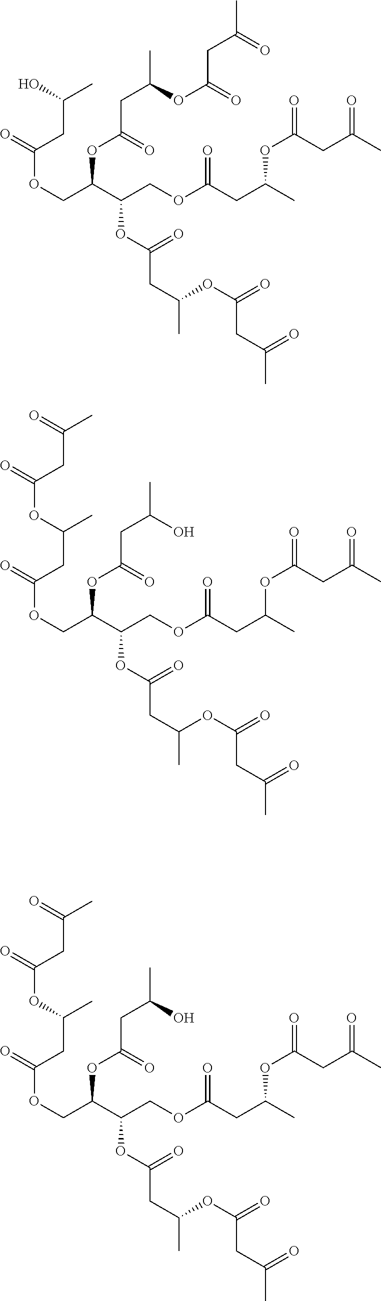 [SMILES: CC(=O)CC(=O)O[C@H](C)CC(=O)OC[C@H](OC(=O)C[C@@H](C)OC(=O)CC(C)=O)[C@@H](COC(=O)C[C@@H](C)O)OC(=O)C[C@@H](C)OC(=O)CC(C)=O ; CC(=O)CC(=O)OC(C)CC(=O)OC[C@H](OC(=O)CC(C)OC(=O)CC(C)=O)[C@@H](COC(=O)CC(C)OC(=O)CC(C)=O)OC(=O)CC(C)O ; CC(=O)CC(=O)O[C@H](C)CC(=O)OC[C@H](OC(=O)C[C@@H](C)OC(=O)CC(C)=O)[C@@H](COC(=O)C[C@@H](C)OC(=O)CC(C)=O)OC(=O)C[C@@H](C)O]

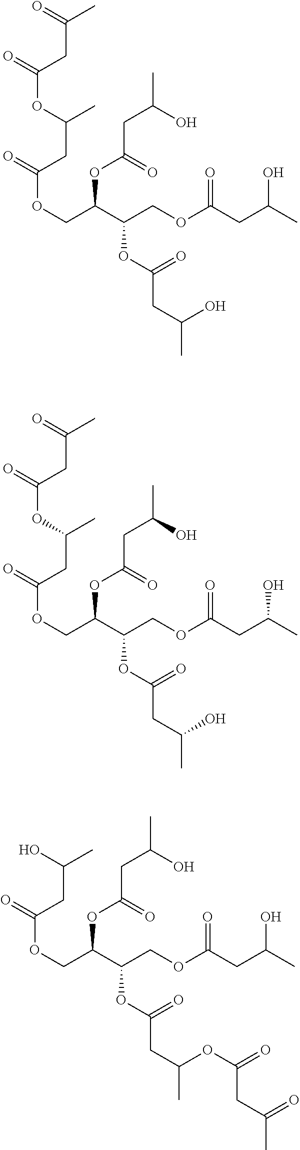 [SMILES: CC(=O)CC(=O)OC(C)CC(=O)OC[C@@H](OC(=O)CC(C)O)[C@H](COC(=O)CC(C)O)OC(=O)CC(C)O ; CC(=O)CC(=O)O[C@H](C)CC(=O)OC[C@@H](OC(=O)C[C@@H](C)O)[C@H](COC(=O)C[C@@H](C)O)OC(=O)C[C@@H](C)O ; CC(=O)CC(=O)OC(C)CC(=O)O[C@@H](COC(=O)CC(C)O)[C@@H](COC(=O)CC(C)O)OC(=O)CC(C)O]

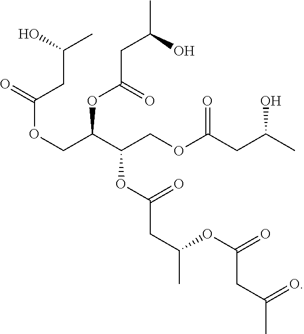 [SMILES: CC(=O)CC(=O)O[C@H](C)CC(=O)O[C@@H](COC(=O)C[C@@H](C)O)[C@@H](COC(=O)C[C@@H](C)O)OC(=O)C[C@@H](C)O]

86. A compound of formula 9

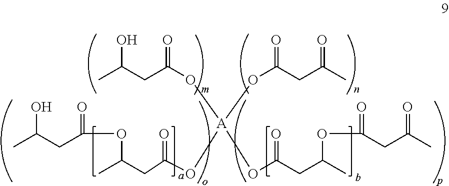

wherein
A is derived from an organic polyol with at least 3 hydroxyl groups,
m is 0, 1, 2, 3, 4, or 5,
n is 0, 1, 2, 3, 4, or 5,
o is 0, 1, 2, 3, 4, or 5,
p is 0, 1, 2, 3, 4, or 5,
at least two of m, n, o, and p are not 0,
m+n+o+p is from 2 to the number of hydroxyl groups of the initial organic polyol A,
a is 1-10, and
b is 1-10.
87. The compound according to item 86, wherein the organic polyol is selected from a linear or branched C₂₋₁₂ alkyl substituted with at least 3 hydroxyl groups, a C₃₋₈ cycloalkyl substituted with at least 3 hydroxyl groups.
88. The compound according to item 87, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.
89. The compound according to item 87 or 88, wherein the C₃₋₈ cycloalkyl substituted with at least 3 hydroxyl groups is selected from the group consisting of cyclopentanetriol, cyclohexanetriol, cyclopentanetetrol, cyclohexanetetrol.
90. The compound according to item 86, wherein the organic polyol is selected from the group consisting of monosaccharides, sugar alcohols, sugar acids.
91. The compound according to item 90, wherein the monosaccharide is selected from tetroses, pentoses, hexoses, heptoses, preferably wherein the monosaccharide is selected from aldotetroses, ketotetroses, aldopentoses, ketopentoses, aldohexosen, ketohexoses, aldoheptoses, ketoheptoses.
92. The compound according to item 90 or 91, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.
93. The compound according to any one of items 90 to 92, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.
94. The compound according to any one of items 90 to 93, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.
95. The compound according to any one of items 86 to 94, wherein the organic polyol is selected from the group consisting of glycerol, sorbitol, xylitol, mannitol, erythritol, maltitol, glucose, glucitol, ribulose, pentaerythritol, trimethylolpropane.
96. The compound according to any one of items 86 to 95, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.
97. The compound according to any one of items 86 to 96, wherein m is 0, 1, 2, or 3.
98. The compound according to any one of items 86 to 97, wherein n is 0, 1, 2, or 3.
99. The compound according to any one of items 86 to 98, wherein o is 0, 1, 2, or 3.
100. The compound according to any one of items 86 to 99, wherein p is 0, 1, 2, or 3.
101. The compound according to any one of items 86 to 100, wherein at least three of m, n, o, and p are not 0.
102. The compound according to any one of items 86 to 101, wherein m+n+o+p is equal to the number of hydroxyl groups of the initial polyol A.
103. The compound according to any one of items 86 to 101, wherein m+n+o+p is less than the number of hydroxyl groups of the initial polyol A.
104. The compound according to any one of items 86 to 103, wherein a is 1-9, such as 1-8, such as 1-7, such as 1-6, such as 1-5, such as 1-4, such as 1-3, or such as 1-2, preferably wherein a is 1 or 2, more preferably wherein a is 1.
105. The compound according to any one of items 86 to 104, wherein b is 1-9, such as 1-8, such as 1-7, such as 1-6, such as 1-5, such as 1-4, such as 1-3, or such as 1-2, preferably wherein b is 1 or 2, more preferably wherein b is 1.
106. The compound according to any one of items 86 to 105, wherein n+p≠a+b+m+o.
107. The compound according to any one of items 86 to 105, wherein the compound is selected from the group consisting of

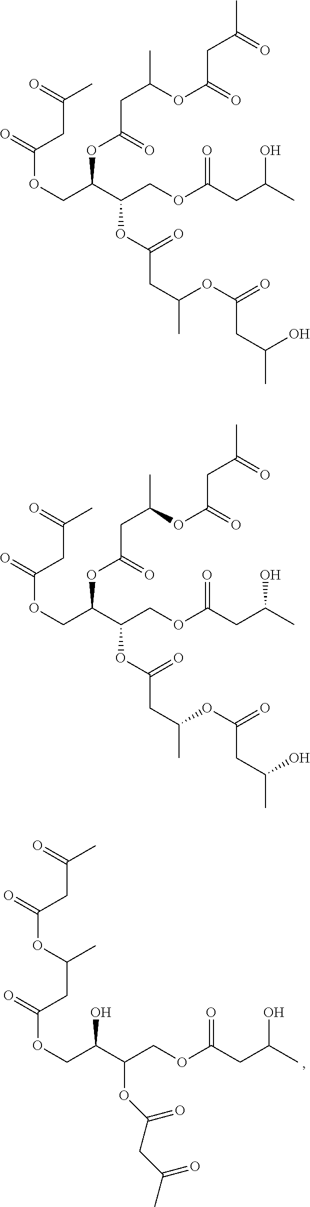 [SMILES: CC(=O)CC(=O)OC[C@@H](OC(=O)CC(C)OC(=O)CC(C)=O)[C@H](COC(=O)CC(C)O)OC(=O)CC(C)OC(=O)CC(C)O ; CC(=O)CC(=O)OC[C@@H](OC(=O)C[C@@H](C)OC(=O)CC(C)=O)[C@H](COC(=O)C[C@@H](C)O)OC(=O)C[C@@H](C)OC(=O)C[C@@H](C)O ; CC(=O)CC(=O)OC(C)CC(=O)OC[C@@H](O)C(COC(=O)CC(C)O)OC(=O)CC(C)=O]

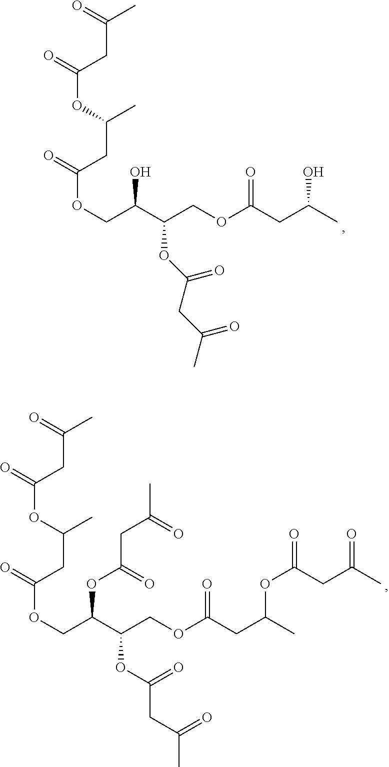 [SMILES: CC(=O)CC(=O)O[C@H](C)CC(=O)OC[C@@H](O)[C@H](COC(=O)C[C@@H](C)O)OC(=O)CC(C)=O ; CC(=O)CC(=O)OC(C)CC(=O)OC[C@H](OC(=O)CC(C)=O)[C@@H](COC(=O)CC(C)OC(=O)CC(C)=O)OC(=O)CC(C)=O]

108. The compound according to any one of items 106 or 107, wherein the compound is selected from the group consisting of

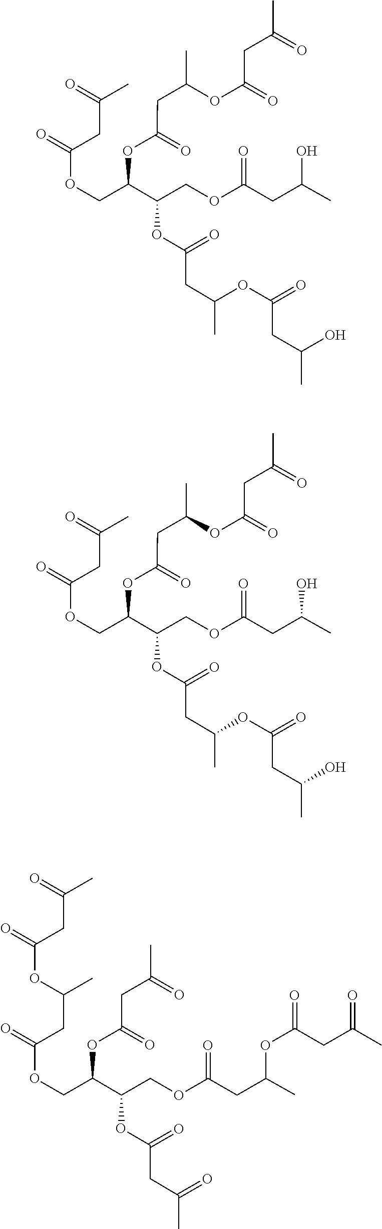 [SMILES: CC(=O)CC(=O)OC[C@@H](OC(=O)CC(C)OC(=O)CC(C)=O)[C@H](COC(=O)CC(C)O)OC(=O)CC(C)OC(=O)CC(C)O ; CC(=O)CC(=O)OC[C@@H](OC(=O)C[C@@H](C)OC(=O)CC(C)=O)[C@H](COC(=O)C[C@@H](C)O)OC(=O)C[C@@H](C)OC(=O)C[C@@H](C)O ; CC(=O)CC(=O)OC(C)CC(=O)OC[C@H](OC(=O)CC(C)=O)[C@@H](COC(=O)CC(C)OC(=O)CC(C)=O)OC(=O)CC(C)=O]

109. The compound according to any one of items 86 to 108, wherein all β-hydroxyl butyric acid ester units are either R-configured or S-configured, or all β-hydroxyl butyric acid ester units are present as a non-racemic mixture of R- and S-configurations.
110. The compound according to any one of items 86 to 109, wherein the compound contains more R-configured β-hydroxyl butyric acid ester units than S-configured β-hydroxyl butyric acid ester units, preferably wherein all β-hydroxyl butyric acid ester units are in R-configuration.
111. A compound of formula 10

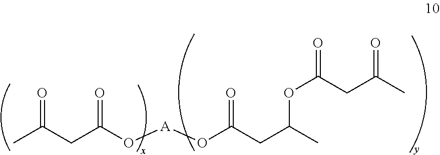

wherein
A is derived from an organic polyol with at least 3 hydroxyl groups,
x is at least 1,
y is at least 1, and
x+y is from 3 to the number of hydroxyl groups of the initial organic polyol A.
112. The compound according to item 111, wherein the organic polyol is selected from a linear or branched C₂₋₁₂ alkyl substituted with at least 3 hydroxyl groups, a C₃₋₈ cycloalkyl substituted with at least 3 hydroxyl groups.
113. The compound according to item 112, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.
114. The compound according to item 112 or 113, wherein the C₃₋₈ cycloalkyl substituted with at least 3 hydroxyl groups is selected from the group consisting of cyclopentanetriol, cyclohexanetriol, cyclopentanetetrol, cyclohexanetetrol.
115. The compound according to item 111, wherein the organic polyol is selected from the group consisting of monosaccharides, sugar alcohols, sugar acids.
116. The compound according to item 115, wherein the monosaccharide is selected from tetroses, pentoses, hexoses, heptoses, preferably wherein the monosaccharide is selected from aldotetroses, ketotetroses, aldopentoses, ketopentoses, aldohexosen, ketohexoses, aldoheptoses, ketoheptoses.
117. The compound according to item 115 or 116, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.
118. The compound according to any one of items 115 to 117, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.
119. The compound according to any one of items 115 to 118, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.
120. The compound according to any one of items 111 to 119, wherein the organic polyol is selected from the group consisting of glycerol, sorbitol, xylitol, mannitol, erythritol, maltitol, glucose, glucitol, ribulose, pentaerythritol, trimethylolpropane.
121. The compound according to any one of items 111 to 120, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.
122. The compound according to any one of items 111 to 121, wherein x+y is equal to the number of hydroxyl groups of the initial polyol A.
123. The compound according to any one of items 111 to 122, wherein the compound is selected from the group consisting of

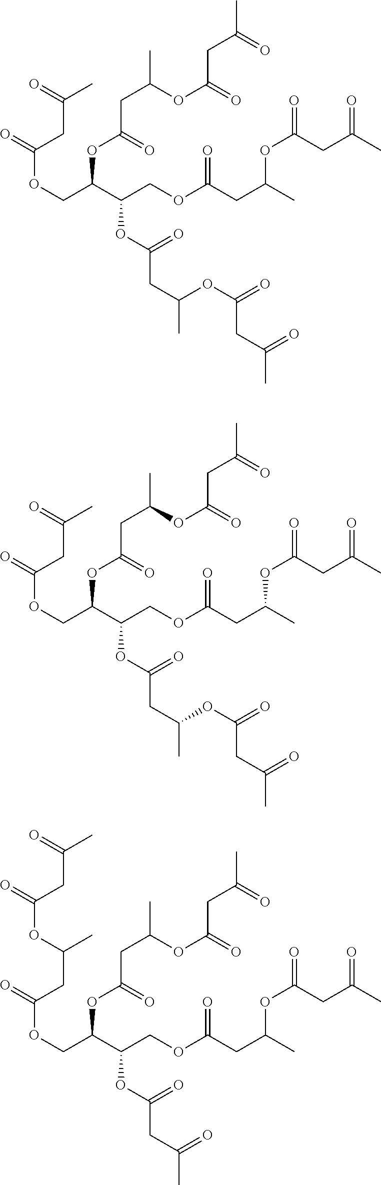 [SMILES: CC(=O)CC(=O)OC[C@@H](OC(=O)CC(C)OC(=O)CC(C)=O)[C@H](COC(=O)CC(C)OC(=O)CC(C)=O)OC(=O)CC(C)OC(=O)CC(C)=O ; CC(=O)CC(=O)OC[C@@H](OC(=O)C[C@@H](C)OC(=O)CC(C)=O)[C@H](COC(=O)C[C@@H](C)OC(=O)CC(C)=O)OC(=O)C[C@@H](C)OC(=O)CC(C)=O ; CC(=O)CC(=O)OC(C)CC(=O)OC[C@H](OC(=O)CC(C)=O)[C@@H](COC(=O)CC(C)OC(=O)CC(C)=O)OC(=O)CC(C)OC(=O)CC(C)=O]

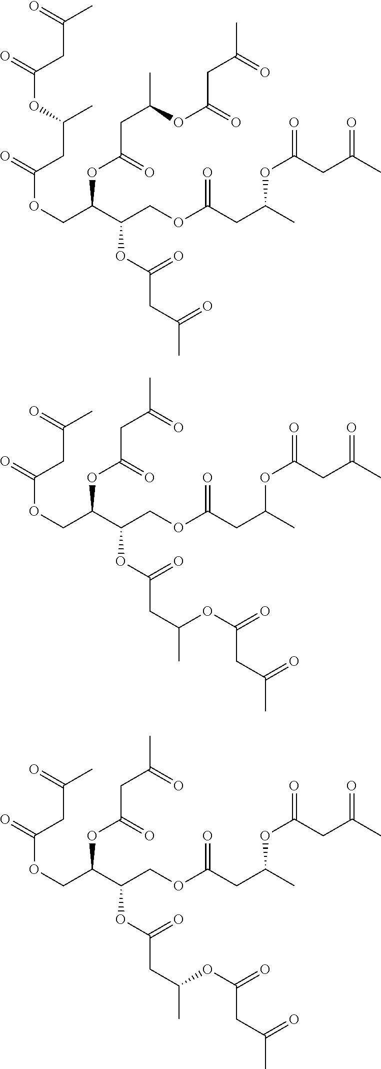

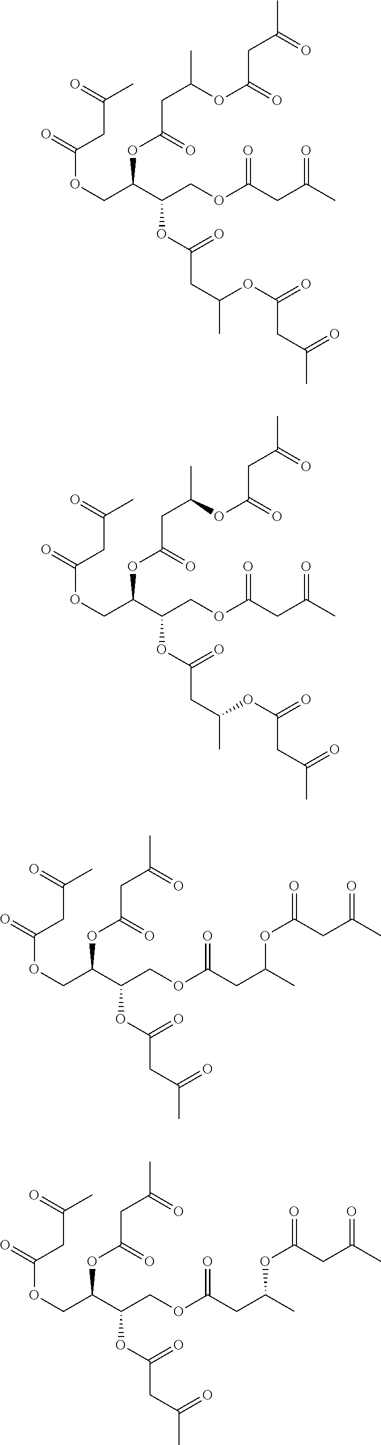

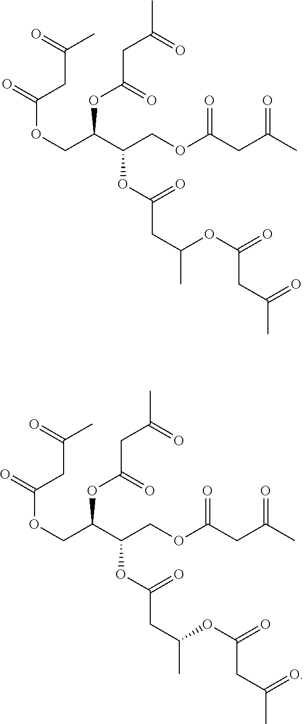 [SMILES: CC(=O)CC(=O)OC[C@H](OC(=O)CC(C)OC(=O)CC(C)=O)[C@@H](COC(=O)CC(C)=O)OC(=O)CC(C)=O ; CC(=O)CC(=O)OC[C@H](OC(=O)C[C@@H](C)OC(=O)CC(C)=O)[C@@H](COC(=O)CC(C)=O)OC(=O)CC(C)=O]

124. The compound according to any one of items 111 to 123, wherein all β-hydroxyl butyric acid ester units are either R-configured or S-configured, or all β-hydroxyl butyric acid ester units are present as a non-racemic mixture of R- and S-configurations.
125. The compound according to any one of items 111 to 124, wherein the compound contains more R-configured β-hydroxyl butyric acid ester units than S-configured β-hydroxyl butyric acid ester units, preferably wherein all β-hydroxyl butyric acid ester units are in R-configuration.
126. A process for the preparation of a compound of formula 10

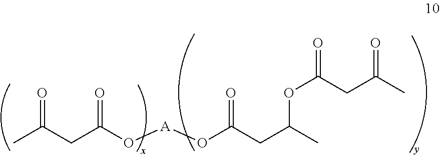

wherein
A is derived from an organic polyol with at least 3 hydroxyl groups,
x is at least 1,
y is at least 1, and
x+y is from 3 to the number of hydroxyl groups of the initial organic polyol A;
wherein the process comprises:
   (i) partially reacting an organic polyol of formula 2 with diketene 3 resulting in the formation of a compound according to formula 11;

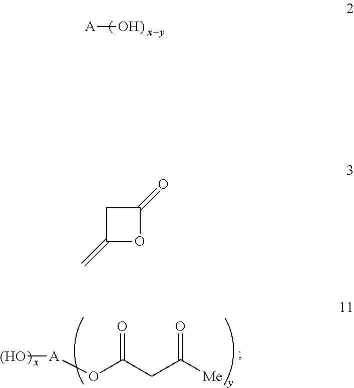

(ii) reacting the compound of formula 11 with hydrogen in the presence of a catalyst resulting in the formation of the compound according to formula 12

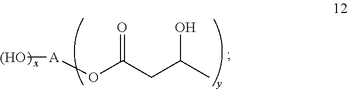

and
(iii) reacting the compound of formula 12 with diketene 3 resulting in the formation of the compound according to formula 10.
127. The process according to item 126, wherein the organic polyol is selected from a linear or branched C₂₋₁₂ alkyl substituted with at least 3 hydroxyl groups, a C₃₋₈ cycloalkyl substituted with at least 3 hydroxyl groups.
128. The process according to item 127, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.
129. The process according to item 127 or 128, wherein the C₃₋₈ cycloalkyl substituted with at least 3 hydroxyl groups is selected from the group consisting of cyclopentanetriol, cyclohexanetriol, cyclopentanetetrol, cyclohexanetetrol.
130. The process according to item 126, wherein the organic polyol is selected from the group consisting of monosaccharides, sugar alcohols, sugar acids.
131. The process according to item 130, wherein the monosaccharide is selected from tetroses, pentoses, hexoses, heptoses, preferably wherein the monosaccharide is selected from aldotetroses, ketotetroses, aldopentoses, ketopentoses, aldohexosen, ketohexoses, aldoheptoses, ketoheptoses.
132. The process according to item 130 or 131, wherein the monosaccharide is selected from the group consisting of erythrose, threose, erythrulose, ribose, arabinose, xylose, lyxose, desoxyribose, ketopentose, ribulose, xylulose, allose, altrose, glucose, mannose, gulose, idose, galactose, talose, n-acetyl-d-glucosamin, glucosamin, N-acetyl-D-galactosamin, fucose, rhamnose, chinovose, fructose, 2-desoxy-D-glucose, fluordesoxyglucose, 6-desoxyfructose, 1,6-dichlorfructose, 3,6-anhydrogalactose, 1-O-methylgalactose, 1-O-methyl-D-glucose, 1-O-methyl-D-fructose, 3-O-methyl-D-fructose, 6-O-methyl-D-galactose, sedoheptulose, mannoheptulose, L-glycero-D-manno-heptose.
133. The process according to any one of items 130 to 132, wherein the sugar alcohol is selected from the group consisting of erythritol, threitol, arabitol, xylitol, ribitol, mannitol, sorbitol, galactitol, fucitol, iditol, inositol, volemitol, isomalt, maltitol, lactitol.
134. The process according to any one of items 130 to 133, wherein the sugar acid is selected from the group consisting of xylonic acid, gluconic acid, ascorbic acid, neuraminic acid, ketodeoxyoctonic acid, glucuronic acid, galacturonic acid, iduronic acid, mucic acid, saccharic acid.
135. The process according to any one of items 126 to 134, wherein the organic polyol is selected from the group consisting of glycerol, sorbitol, xylitol, mannitol, erythritol, maltitol, glucose, glucitol, ribulose, pentaerythritol, trimethylolpropane.
136. The process according to any one of items 126 to 135, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.
137. The process according to any one of items 126 to 136, wherein x+y is equal to the number of hydroxyl groups of the initial polyol A.
138. The process according to any one of items 126 to 137, wherein reaction step (i) is performed in the presence of an organic amine catalyst.
139. The process according to any one of items 126 to 138, wherein reaction step (iii) is performed in the presence of an organic amine catalyst.
140. The process according to item 138 or 139, wherein the organic amine catalyst is a tertiary amine.
141. The process according to item 140, wherein the organic amine catalyst is DABCO.
142. The process according to any one of items 126 to 141, wherein reaction step (ii) is performed in the presence of a metal-based catalyst, preferably a Ni-based catalyst, a Pd-based catalyst, a Pt-based catalyst, a Ru-based catalyst, a Co-based catalyst, an Ir-based catalyst, or a Rh-based catalyst.
143. The process according to any one of items 126 to 142, wherein reaction step (ii) is performed in the presence of a Ru-based catalyst, preferably selected from a Ruthenium oxide catalyst, Ru/C, RuAl₂O₃, RuO₂, Ru(OAc)₂(BINAP), Ru(Cl)₂(BINAP), C3-[(S,S)-teth-MtsDpenRuCl], [(R)-BinapRuCl(p-cymene)]Cl, and [Chloro(R)-C3-TunePhos)(p-cymene)ruthenium(II)] chloride.
144. The process according to item 142 or 143, wherein reaction step (ii) is performed in the presence of a chiral ligand capable of forming complexes with the metal-based catalyst, preferably wherein the chiral ligand is selected from the group consisting of 2,2′-bis(diphenylphosphino)-1,1′-binaphthyl (BINAP), 1,1′-Bi-2-naphthol (BINOL), 2,3-O-isopropylidene-2,3-dihydroxy-1,4-bis(diphenylphosphino) butane (DIOP), 2,2′,5,5′-tetramethyl-4,4′-bis-(diphenylphoshino)-3,3′-bithiophene (tetraMe-BITIOP), Bis(diphenylphosphino)-7,8-dihydro-6H-dibenzo[f,h][1,5]dioxonin (C3-TunePhos), 4,4′-Bis(bis(3,5-dimethylphenyl)phosphino)-2,2′,6,6′-tetramethoxy-3,3′-bipyridine (Xyl-p-PHOS), (6,6′-Dimethoxybiphenyl-2,2′-diyl)-bis-(diphenylphosphin) (MeO-BIPHEP), and 1,2-Bis[(2-methoxyphenyl)phenylphosphino]ethane (DIPAMP).
145. The process according to any one of items 126 to 144, wherein all β-hydroxyl butyric acid ester units are either R-configured or S-configured, or all β-hydroxyl butyric acid ester units are present as a non-racemic mixture of R- and S-configurations.
146. The process according to any one of items 126 to 145, wherein the compound contains more R-configured β-hydroxyl butyric acid ester units than S-configured β-hydroxyl butyric acid ester units, preferably wherein all β-hydroxyl butyric acid ester units are in R-configuration.
147. The process according to any one of items 126 to 146, wherein the compound is selected from the group consisting of

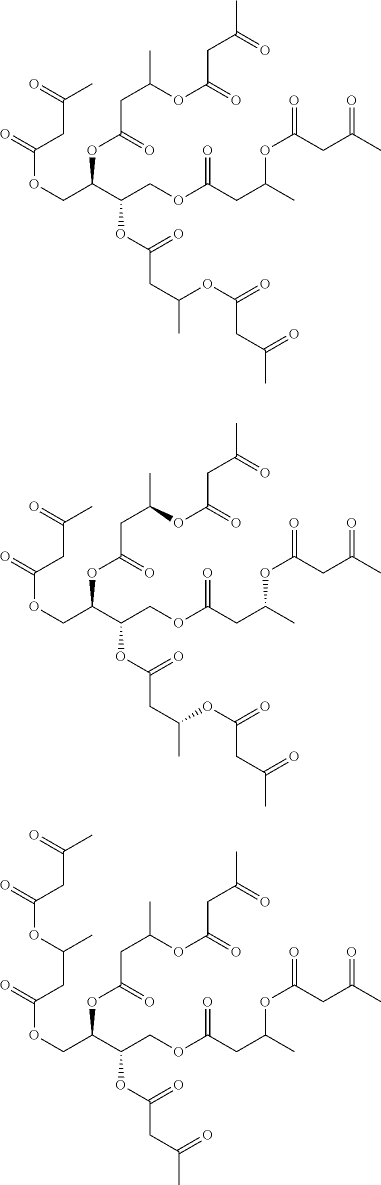 [SMILES: CC(=O)CC(=O)OC[C@@H](OC(=O)CC(C)OC(=O)CC(C)=O)[C@H](COC(=O)CC(C)OC(=O)CC(C)=O)OC(=O)CC(C)OC(=O)CC(C)=O ; CC(=O)CC(=O)OC[C@@H](OC(=O)C[C@@H](C)OC(=O)CC(C)=O)[C@H](COC(=O)C[C@@H](C)OC(=O)CC(C)=O)OC(=O)C[C@@H](C)OC(=O)CC(C)=O ; CC(=O)CC(=O)OC(C)CC(=O)OC[C@H](OC(=O)CC(C)=O)[C@@H](COC(=O)CC(C)OC(=O)CC(C)=O)OC(=O)CC(C)OC(=O)CC(C)=O]

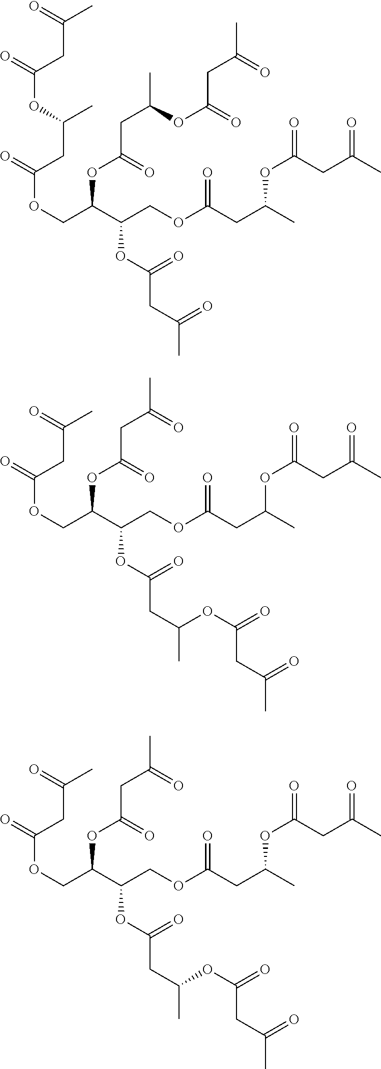

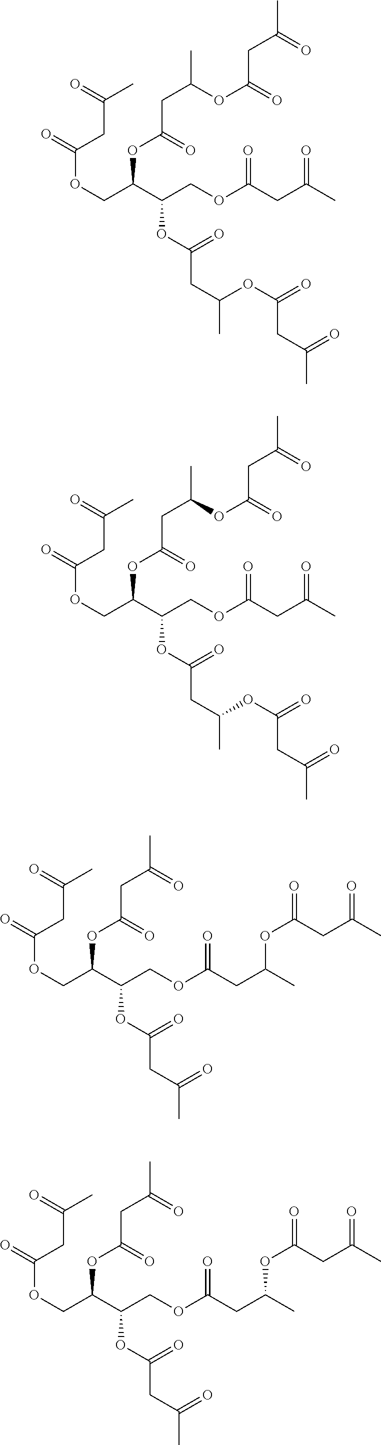

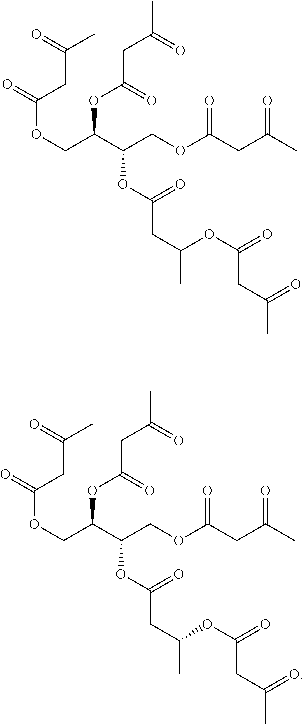 [SMILES: CC(=O)CC(=O)OC[C@H](OC(=O)CC(C)OC(=O)CC(C)=O)[C@@H](COC(=O)CC(C)=O)OC(=O)CC(C)=O ; CC(=O)CC(=O)OC[C@H](OC(=O)C[C@@H](C)OC(=O)CC(C)=O)[C@@H](COC(=O)CC(C)=O)OC(=O)CC(C)=O]

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention. All embodiments and preferred embodiments described herein in connection with one particular aspect of the invention (e.g. the inventive preservative composition) shall likewise apply to all other aspects of the present inventions such as end-use formulations, uses or methods according to the present invention.

The present invention will be further illustrated by the following examples.

### EXAMPLES

### Example 1

Meso-Erythritol (6 g, 0.05 mol, 1 eq.) was introduced into a stirred tank reactor and ethyl acetate (10.8 g, 2.5 eq.) was added. DABCO (7.2 mg, 0.0001 mol, 0.0013 eq.) was added to the suspension. Subsequently, diketene (4.1 g, 0.05 mol, 1 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of meso-erythritol monoacetoacetate (7.2 g, 72%) as a white-yellow solid. 1H NMR (400 MHz, DMSO-d₆) δ ppm 2.18 (m, 3H), 3.39 (s, 5H), 4.35 (m, 2H), 4.48 (s, 2H).

### Example 2

Meso-Erythritol (6 g, 0.05 mol, 1 eq.) was introduced into a stirred tank reactor and ethyl acetate (10.8 g, 2.5 eq.) was added. DABCO (7.2 mg, 0.0001 mol, 0.0013 eq.) was added to the suspension. Subsequently, diketene (8.3 g, 0.1 mol, 2 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of meso-erythritol diacetoacetate (13.4 g, 94%) as an orange solid. 1H NMR (400 MHz, DMSO-d₆) δ ppm 2.18 (m, 6H), 3.38 (m, 6H), 3.54 (m, 2H), 4.24 (m, 1H), 4.43 (m, 1H), 4.48 (s, 1H), 5.52 (m, 1H).

### Example 3

Meso-Erythritol (6 g, 0.05 mol, 1 eq.) was introduced into a stirred tank reactor and ethyl acetate (10.8 g, 2.5 eq.) was added. DABCO (7.2 mg, 0.0001 mol, 0.0013 eq.) was added to the suspension. Subsequently, diketene (12.4 g, 0.15 mol, 3 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of meso-erythritol triacetoacetate (18.1 g, 99%) as a yellow suspension. 1H NMR (400 MHz, DMSO-d₆) δ ppm 2.18 (m, 9H), 3.37 (m, 7H), 3.61 (m, 3H), 3.65 (m, 3H), 4.23 (m, 1H), 4.35 (m, 2H), 5.25 (m, 1H).

### Example 4

Xylitol (13.0 g, 0.09 mol, 1 eq.) was introduced into a stirred tank reactor and ethyl acetate (18.8 g, 2.5 eq.) was added. DABCO (7.2 mg, 0.0001 mol, 0.0013 eq.) was added to the suspension. Subsequently, diketene (7.2 g, 0.09 mol, 1 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of xylitol monoacetoacetate (20.3 g, 95%) as a yellow solid. 1H NMR (400 MHz, DMSO-d₆) δ ppm 2.18 (m, 3H), 3.37 (m, 4H), 3.44 (m, 3H), 3.54 (m, 4H), 4.44 (m, 8H).

### Example 5

Xylitol (13.0 g, 0.09 mol, 1 eq.) was introduced into a stirred tank reactor and ethyl acetate (18.8 g, 2.5 eq.) was added. DABCO (7.2 mg, 0.0001 mol, 0.0013 eq.) was added to the suspension. Subsequently, diketene (14.4 g, 0.17 mol, 2 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of xylitol diacetoacetate (26.4 g, 92%) as an orange solid. 1H NMR (400 MHz, DMSO-d₆) δ ppm 2.19 (m, 6H), 3.37 (m, 6H), 3.44 (m, 9H), 3.54 (m, 6H), 4.44 (m, 9H).

### Example 6

Xylitol (13.0 g, 0.09 mol, 1 eq.) was introduced into a stirred tank reactor and ethyl acetate (18.8 g, 2.5 eq.) was added. DABCO (7.2 mg, 0.0001 mol, 0.0013 eq.) was added to the suspension. Subsequently, diketene (21.5 g, 0.26 mol, 3 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of xylitol triacetoacetate (31.7 g, 87%) as an orange solid. 1H NMR (400 MHz, DMSO-d₆) δ ppm 2.19 (m, 9H), 3.37 (m, 5H), 3.45 (m, 9H), 3.53 (m, 6H), 4.45 (m, 8H).

### Example 7

Xylitol (13.0 g, 0.09 mol, 1 eq.) was introduced into a stirred tank reactor and ethyl acetate (18.8 g, 2.5 eq.) was added. DABCO (7.2 mg, 0.0001 mol, 0.0013 eq.) was added to the suspension. Subsequently, diketene (28.7 g, 0.34 mol, 4 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of xylitol tetraacetoacetate (39.6 g, 90%) as an orange suspension. 1H NMR (400 MHz, DMSO-d₆) δ ppm 2.19 (m, 9H), 3.36 (m, 4H), 3.45 (m, 5H), 3.53 (m, 4H), 6.62 (m, 4H), 3.69 (m, 2H), 3.75 (s, 1H), 4.45 (m, 5H).

### Example 8

(2R,3S)-butane-1,2,3,4-tetrayl tetrakis(3-oxobutanoate) (50 g, 110 mmol, 1 eq.) was placed in an autoclave and ethyl acetate (192 g, 20 eq.) was added. Then catalyst (RuO₂, 132 mg, 1.1 mmol, 0.01 eq.) was added and the atmosphere was exchanged by pressurizing the reactor three times with nitrogen, followed by pressurizing three times with hydrogen. The hydrogen pressure was adjusted to 20 bar and the mixture was heated to 60° C. with 600 rpm stirring until the desired hydrogen uptake was observed. After 1 eq. and 2 eq. of hydrogen uptake 50 ml of the reaction mixture were taken. After 3 eq. of hydrogen uptake the hydrogenation was stopped. Subsequently the samples were cooled to room temperature and the hydrogen atmosphere was exchanged with nitrogen. The reaction mixture was mixed with activated charcoal and filtered over celite. The solvent was evaporated from the filtrate and the product was analyzed. A total of three different mixtures could be isolated.

A mixture of isomers of the one times hydrogenated (2R,3S)-butane-1,2,3,4-tetrayl tetrakis(3-oxobutanoate) was isolated as a yellow oil (9.33 g). 1H NMR (400 MHz, DMSO-d₆) δ ppm 0.97-1.13 (m, 3H), 2.18 (s, 9H), 2.56 (m, 2H), 3.60 (m, 6H), 4.24 (s, 2H), 4.36 (m, 2H), 5.25 (m, 2H).

A mixture of isomers of the two times hydrogenated (2R,3S)-butane-1,2,3,4-tetrayl tetrakis(3-oxobutanoate) was isolated as a yellow oil (6.43 g). 1H NMR (400 MHz, DMSO-d₆) δ ppm 1.09 (m, 6H), 2.18 (m, 4H), 3.37 (m, 4H), 3.62 (m, 3H), 3.99 (m, 2H), 4.16 (m, 1H), 4.33 (m, 1H), 4.75 (m, 2H).

A mixture of isomers of the three times hydrogenated (2R,3S)-butane-1,2,3,4-tetrayl tetrakis(3-oxobutanoate) was isolated as a yellow oil (35.2 g). 1H NMR (400 MHz, DMSO-d₆) δ ppm 1.09 (m, 9H), 2.16 (m, 2H), 2.36 (m, 6H), 3.60 (m, 1H), 4.00 (m, 4H), 4.16 (m, 1H), 4.32 (m, 1H), 4.75 (m, 2H).

### Example 9

A mixture of isomers of meso-erythritol monoacetoacetate (6.9 g, 0.03 mol, 1 eq., Example 1) was placed in an autoclave with ethyl acetate (141 g, 41 eq.). RuO₂ (0.08 g, 0.6 mmol, 0.02 eq.) was added and the atmosphere was exchanged by pressurizing the reactor three times with nitrogen, followed by pressurizing three times with hydrogen. The hydrogen pressure was adjusted to 20 bar and the mixture was heated to 60° C. with 1000 rpm stirring the possible hydrogen uptake was observed (6d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of meso-erythritol mono(3-hydroxybutanoate) was isolated as a yellow oil (3.42 g, 49%). 1H NMR (400 MHz, DMSO-d₆) δ ppm 1.09 (m, 3H), 2.36 (m, 2H), 3.36 (m, 3H), 3.94 (m, 3H), 4.30 (m, 3H).

### Example 10

A mixture of isomers of meso-erythritol diacetoacetate (11.9 g, 0.04 mol, 1 eq., Example 2) was placed in an autoclave with ethyl acetate (141 g, 41 eq.). Ru/C (5 wt %, 4 g, 2.0 mmol, 0.05 eq.) was added and the atmosphere was exchanged by pressurizing the reactor three times with nitrogen, followed by pressurizing three times with hydrogen. The hydrogen pressure was adjusted to 10 bar and the mixture was heated to 40° C. with 1000 rpm stirring the possible hydrogen uptake was observed (1d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of meso-erythritol di(3-hydroxybutanoate) was isolated as a yellow oil (7.51 g, 64%). 1H NMR (400 MHz, DMSO-d₆) δ ppm 1.10 (m, 6H), 2.36 (m, 4H), 4.01 (m, 3H), 4.17 (m, 1H), 4.34 (m, 1H), 4.74 (m, 2H).

### Example 11

A mixture of isomers of meso-erythritol triacetoacetate (16.5 g, 0.04 mol, 1 eq., Example 3) was placed in an autoclave with ethyl acetate (140 g, 36 eq.). Ru/C (5 wt %, 5.5 g, 2.7 mmol, 0.06 eq.) was added and the atmosphere was exchanged by pressurizing the reactor three times with nitrogen, followed by pressurizing three times with hydrogen. The hydrogen pressure was adjusted to 10 bar and the mixture was heated to 40° C. with 1000 rpm stirring the possible hydrogen uptake was observed (1d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of meso-erythritol tri (3-hydroxybutanoate) was isolated as a yellow oil (16.6 g, 99%). 1H NMR (400 MHz, DMSO-d₆) δ ppm 1.09 (m, 9H), 2.37 (m, 6H), 3.99 (m, 3H) 4.15 (m, 1H), 4.31 (m, 1H), 4.74 (m, 3H).

### Example 12

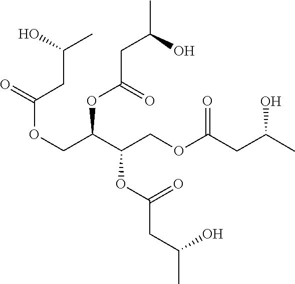 [SMILES: C[C@@H](O)CC(=O)OC[C@H](OC(=O)C[C@@H](C)O)[C@@H](COC(=O)C[C@@H](C)O)OC(=O)C[C@@H](C)O]

(2R,3S)-butane-1,2,3,4-tetrayl tetrakis(3-oxobutanoate) (25 g, 54.5 mmol, 1 eq.) was placed in an autoclave and methanol (61.2 g, 35 eq.) was added. Then catalyst ([RuCl₂((R)-BINAP)]₂NEt₃ (218 mg, 0.1 mmol, 0.002 eq.) and H₂SO₄ (1M, 97 mg, 0.002 eq.) was added and the atmosphere was exchanged by pressurizing the reactor three times with nitrogen, followed by pressurizing three times with hydrogen. The hydrogen pressure was adjusted to 20 bar and the mixture was heated to 60° C. with 600 rpm stirring until no further hydrogen uptake was observed (5 d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The final product (2R,3S)-butane-1,2,3,4-tetrayl (3R,3′R,3″R,3″R)-tetrakis(3-hydroxybutanoate) was isolated as an orange oil (17.2 g, 67.8%). 1H NMR (400 MHz, DMSO-d₆) δ ppm 1.28 (m, 12H), 2.50 (m, 8H), 4.39 (m, 8H), 5.5 (m, 2H).

### Example 13

(2R,3S)-butane-1,2,3,4-tetrayl (3R,3′R,3″R,3′R)-tetrakis(3-hydroxybutanoate) (1.0 g, 2 mmol, 1 eq., Example 12) was introduced into a stirred tank reactor and ethyl acetate (2.0 g, 10 eq.) was added. DABCO (0.3 mg, 0.03 mmol, 0.001 eq.) was added to the suspension. Subsequently, diketene (0.2 g, 2 mmol, 1 eq.) was slowly added while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of the monoesterified (2R,3S)-butane-1,2,3,4-tetrayl (3R,3′R,3″R,3′R)-tetrakis(3-hydroxybutanoate) (0.98 g, 85%) as a viscous brown oil. 1H NMR (400 MHz, DMSO-d₆) δ ppm 1.09 (m, 9H), 1.24 (m, 3H), 2.16 (m, 3H), 2.35 (m, 6H), 2.65 (m, 2H), 3.35 (m, 2H), 3.98 (m, 4H), 4.16 (m, 2H), 4.33 (m, 2H), 5.14 (m, 1H), 5.21 (s, 2H).

### Example 14

(2R,3S)-butane-1,2,3,4-tetrayl (3R,3′R,3″R,3′″R)-tetrakis(3-hydroxybutanoate) (1.0 g, 2 mmol, 1 eq., Example 12) was introduced into a stirred tank reactor and ethyl acetate (2.0 g, 10 eq.) was added. DABCO (0.3 mg, 0.03 mmol, 0.001 eq.) was added to the suspension. Subsequently, diketene (0.4 g, 2 mmol, 2 eq.) was slowly added while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of the diesterified (2R,3S)-butane-1,2,3,4-tetrayl (3R,3′R,3″R,3′R)-tetrakis(3-hydroxybutanoate) (1.0 g, 81%) as a viscous brown oil. 1H NMR (400 MHz, DMSO-d₆) δ ppm 1.09 (m, 12H), 2.14 (m, 3H), 2.36 (m, 8H), 2.65 (m, 2H), 3.35 (m, 2H), 4.00 (m, 5H), 4.17 (m, 2H), 4.32 (m, 2H), 4.75 (m, 4H), 5.21 (s, 1H).

### Example 15

(2R,3S)-butane-1,2,3,4-tetrayl (3R,3′R,3″R,3′R)-tetrakis(3-hydroxybutanoate) (1.0 g, 2 mmol, 1 eq., Example 12) was introduced into a stirred tank reactor and ethyl acetate (2.0 g, 10 eq.) was added. DABCO (0.3 mg, 0.03 mmol, 0.001 eq.) was added to the suspension. Subsequently, diketene (0.6 g, 6 mmol, 3 eq.) was slowly added while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of the triesterified (2R,3S)-butane-1,2,3,4-tetrayl (3R,3′R,3″R,3′R)-tetrakis(3-hydroxybutanoate) (1.3 g, 93%) as a viscous brown oil. 1H NMR (400 MHz, DMSO-d₆) δ ppm 1.18 (m, 12H), 2.16 (m, 11H), 2.67 (m, 6H), 2.65 (m, 2H), 3.53 (m, 6H), 3.99 (m, 1H), 4.16 (m, 2H), 4.32 (m, 2H), 4.36 (m, 2H), 5.20 (m, 5H).

### Example 16

(2R,3S)-butane-1,2,3,4-tetrayl tetrakis(3-oxobutanoate) (50 g, 109 mmol, 1 eq.) was placed in an autoclave and methanol (110 ml, 25 eq.) was added. Then catalyst ([RuCl₂((R)-BINAP)]₂NEt₃, 397 mg, 0.2 mmol, 0.002 eq.) and H₂SO₄ (1M, 106 mg, 0.002 eq.) was added and the atmosphere was exchanged by pressurizing the reactor three times with nitrogen, followed by pressurizing three times with hydrogen. The hydrogen pressure was adjusted to 20 bar and the mixture was heated to 60° C. with 600 rpm stirring until 50% of the hydrogen uptake was observed (11d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. A mixture of (R)-isomers of the two times hydrogenated (2R,3S)-butane-1,2,3,4-tetrayl tetrakis(3-oxobutanoate) was isolated as an orange oil (42 g, 83%). 1H NMR (400 MHz, DMSO-d₆) δ ppm 1.19 (m, 6H), 2.23 (m, 5H), 2.43 (m, 4H), 3.5 (m, 4H), 4.17 (m, 4H), 5.09 (m, 1H), 5.3 (m, 1H).

### Example 17

A mixture of isomers of xylitol monoacetoacetate (14.5 g, 0.06 mol, 1 eq., Example 4) was placed in an autoclave with ethyl acetate (110 g, 20 eq.). Ru/C (5 wt %, 4 g, 1.8 mmol, 0.03 eq.) was added and the atmosphere was exchanged by pressurizing the reactor three times with nitrogen, followed by pressurizing three times with hydrogen. The hydrogen pressure was adjusted to 10 bar and the mixture was heated to 40° C. with 1000 rpm stirring the possible hydrogen uptake was observed (1d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of xylitol mono(3-hydroxybutanoate) was isolated as a yellow oil (5.6 g, 39%). 1H NMR (400 MHz, DMSO-d₆) δ ppm 1.09 (m, 3H), 2.36 (m, 2H), 4.17 (m, 3H) 4.76 (m, 1H).

### Example 18

A mixture of isomers of xylitol diacetoacetate (16.6 g, 0.06 mol, 1 eq., Example 5) was placed in an autoclave with ethyl acetate (95 g, 18 eq.). Ru/C (5 wt %, 3.1 g, 1.5 mmol, 0.03 eq.) was added and the atmosphere was exchanged by pressurizing the reactor three times with nitrogen, followed by pressurizing three times with hydrogen. The hydrogen pressure was adjusted to 10 bar and the mixture was heated to 40° C. with 1000 rpm stirring the possible hydrogen uptake was observed (2d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of xylitol di(3-hydroxybutanoate) was isolated as a yellow oil (8.9 g, 39%). 1H NMR (400 MHz, DMSO-d₆) δ ppm 1.10 (m, 6H), 2.35 (m, 4H), 3.97 (m, 2H) 4.17 (m, 1H), 4.40 (m, 1H), 4.76 (m, 2H).

### Example 19

A mixture of isomers of xylitol triacetoacetate (17.1 g, 0.06 mol, 1 eq., Example 6) was placed in an autoclave with ethyl acetate (100 g, 19 eq.). Ru/C (5 wt %, 2.6 g, 1.3 mmol, 0.02 eq.) was added and the atmosphere was exchanged by pressurizing the reactor three times with nitrogen, followed by pressurizing three times with hydrogen. The hydrogen pressure was adjusted to 10 bar and the mixture was heated to 40° C. with 1000 rpm stirring the possible hydrogen uptake was observed (2d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of xylitol tri (3-hydroxybutanoate) was isolated as a yellow oil (11.1 g, 45%). 1H NMR (400 MHz, DMSO-d₆) δ ppm 1.09 (m, 9H), 2.35 (m, 6H), 3.97 (m, 3H) 4.18 (m, 1H), 4.39 (m, 1H), 4.75 (m, 3H).

### Example 20

A mixture of isomers of xylitol tetraacetoacetate (29.2 g, 0.06 mol, 1 eq., Example 7) was placed in an autoclave with ethyl acetate (105 g, 20 eq.). Ru/C (5 wt %, 2.92 g, 1.4 mmol, 0.02 eq.) was added and the atmosphere was exchanged by pressurizing the reactor three times with nitrogen, followed by pressurizing three times with hydrogen. The hydrogen pressure was adjusted to 10 bar and the mixture was heated to 40° C. with 1000 rpm stirring the possible hydrogen uptake was observed (4d). Subsequently the mixture was cooled to room temperature and the hydrogen atmosphere was exchanged with nitrogen. The reaction mixture was mixed with activated charcoal and filtered over celite. The solvent was evaporated from the filtrate and the product was analyzed. The obtained mixture of isomers of xylitol tetrakis(3-hydroxybutanoate) was isolated as a yellow oil (21.3 g, 73%). 1H NMR (400 MHz, DMSO-d₆) δ ppm 1.10 (m, 12H), 2.35 (m, 8H), 3.98 (m, 4H) 4.17 (m, 2H), 4.38 (m, 1H), 4.76 (m, 4H).

### Example 21

(2R,3S)-butane-1,2,3,4-tetrayl tetrakis(3-hydroxybutanoate) (24.0 g, 0.05 mol, 1 eq.) was introduced into a stirred tank reactor and ethyl acetate (18.1 g, 4 eq.) was added. DABCO (0.8 mg, 0.07 mmol, 0.001 eq.) was added to the suspension. Subsequently, diketene (4.4 g, 0.05 mol, 1 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of the monoesterified (2R,3S)-butane-1,2,3,4-tetrayl tetrakis(3-hydroxybutanoate) (30.0 g, 106%) as a yellow oil. 1H NMR (400 MHz, DMSO-d₆) δ ppm 1.08 (m, 9H), 1.24 (m, 3H), 2.16 (m, 3H), 2.35 (m, 5H), 2.65 (m, 2H), 3.99 (m, 4H), 4.15 (m, 2H), 4.30 (m, 2H), 4.74 (m, 3H), 5.13 (m, 1H), 5.21 (s, 2H).

### Example 22

(2R,3S)-butane-1,2,3,4-tetrayl tetrakis(3-hydroxybutanoate) (24.0 g, 0.05 mol, 1 eq.) was introduced into a stirred tank reactor and ethyl acetate (18.1 g, 4 eq.) was added. DABCO (0.8 mg, 0.07 mmol, 0.001 eq.) was added to the suspension. Subsequently, diketene (8.7 g, 0.10 mol, 2 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of the diesterified (2R,3S)-butane-1,2,3,4-tetrayl tetrakis(3-hydroxybutanoate) (34.2 g, 103%) as a yellow oil. 1H NMR (400 MHz, DMSO-d₆) δ ppm 1.09 (m, 6H), 1.25 (m, 6H), 2.17 (m, 6H), 2.35 (m, 4H), 2.65 (m, 4H), 3.54 (m, 4H), 3.98 (m, 2H), 4.15 (s, 2H), 4.31 (m, 2H), 4.74 (m, 2H), 5.12 (m, 2H), 5.22 (s, 2H).

### Example 23

(2R,3S)-butane-1,2,3,4-tetrayl tetrakis(3-hydroxybutanoate) (24.0 g, 0.05 mol, 1 eq.) was introduced into a stirred tank reactor and ethyl acetate (18.1 g, 4 eq.) was added. DABCO (0.8 mg, 0.07 mmol, 0.001 eq.) was added to the suspension. Subsequently, diketene (13.0 g, 0.15 mol, 3 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of the triesterified (2R,3S)-butane-1,2,3,4-tetrayl tetrakis(3-hydroxybutanoate) (36.8 g, 100%) as a yellow oil. 1H NMR (400 MHz, DMSO-d₆) δ ppm 1.09 (m, 3H), 1.24 (m, 9H), 2.16 (m, 9H), 2.36 (m, 2H), 2.64 (m, 6H), 3.54 (m, 4H), 3.98 (m, 1H), 4.16 (s, 2H), 4.33 (m, 2H), 4.75 (m, 1H), 5.13 (m, 3H), 5.22 (s, 2H).

### Example 24

The mixture of isomers of meso-erythritol mono(3-hydroxybutanoate) obtained in Example 9 (2.0 g, 0.01 mol, 1 eq.) was introduced into a stirred tank reactor and ethyl acetate (2.5 ml, 2.5 eq.) was added. DABCO (1.4 mg, 0.1 mmol, 0.0013 eq.) was added to the suspension. Subsequently, diketene (0.8 g, 0.01 mol, 1 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of the monoesterified Example 9 (2.8 g, 96%) as a yellow oil. 1H NMR (400 MHz, DMSO-d₆) δ ppm 1.24 (m, 3H), 2.16 (s, 3H), 2.65 (m, 2H), 3.59 (m, 5H), 4.00-4.40 (m, 5H), 5.24 (m, 1H).

### Example 25

The mixture of isomers of meso-erythritol di(3-hydroxybutanoate) obtained in Example 10 (3.0 g, 0.01 mol, 1 eq.) was introduced into a stirred tank reactor and ethyl acetate (2.5 ml, 2.5 eq.) was added. DABCO (1.5 mg, 0.1 mmol, 0.0013 eq.) was added to the suspension. Subsequently, diketene (1.7 g, 0.02 mol, 2 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of the diesterified Example 10 (4.3 g, 93%) as a yellow oil. 1H NMR (400 MHz, DMSO-d₆) δ ppm 1.23 (m, 6H), 2.15 (s, 6H), 2.65 (m, 3H), 3.54 (m, 3H), 3.64 (m, 1H), 4.00 (m, 1H), 4.17 (m, 1H), 4.37 (m, 1H), 5.18 (m, 2H).

### Example 26

The mixture of isomers of meso-erythritol tri (3-hydroxybutanoate) obtained in Example 11 (3.0 g, 7.9 mmol, 1 eq.) was introduced into a stirred tank reactor and ethyl acetate (2 ml, 2.5 eq.) was added. DABCO (1.5 mg, 0.1 mmol, 0.0013 eq.) was added to the suspension. Subsequently, diketene (2.0 g, 0.02 mol, 3 eq.) was slowly dosed to the reaction mixture over 8 h while cooling the reactor jacket to maintain an internal temperature of 40° C. The dosing rate was adjusted in order to maintain an internal temperature of 40° C. After complete addition the mixture was maintained at an internal temperature of 40° C. overnight. The solvent was removed under reduced pressure to obtain a mixture of isomers of the triesterified Example 11 (4.7 g, 95%) as a yellow oil. 1H NMR (400 MHz, DMSO-d₆) δ ppm 1.24 (m, 9H), 2.16 (m, 9H), 2.66 (m, 6H), 3.54 (s, 5H), 3.99 (m, 1H), 4.16 (m, 2H), 4.34 (m, 2H), 5.14 (m, 3H).

### Example 27

(2R,3R,4S)-pentane-1,2,3,4,5-pentayl pentakis (3-hydroxybutanoate) (10 g, 50 mmol, 1 eq.) was introduced into a stirred tank reactor and ethyl acetate (10.6 g, 3 eq.) was added. DABCO (7.1 mg, 0.1 mmol, 0.0013 eq.) was added to the suspension. Subsequently, diketene (4 g, 50 mmol, 1 eq.) was slowly dosed to the reaction mixture at 50° C. The dosing rate was adjusted in order to maintain an internal temperature of 48-52° C. After complete addition the mixture was maintained at an internal temperature of 50° C. for an additional 12 h and the solvent was evaporated. Finally the reaction mixture was cooled to room temperature and analyzed. A mixture of the monoesterified (2R,3R,4S)-pentane-1,2,3,4,5-pentayl pentakis (3-hydroxybutanoate) was isolated as a yellow-orange oil (13.5 g, 46%). 1H NMR (400 MHz, DMSO-d₆) δ ppm 1.23 (m, 12H), 2.18 (m, 18H) 2.65 (m, 6H), 3.56 (m, 5H), 3.68 (m, 6H), 4.10 (m, 1H), 4.21 (m, 3H), 5.13 (m, 3H), 5.27 (m, 2H), 5.42 (m, 1H).

### Example 28

(2R,3R,4S)-pentane-1,2,3,4,5-pentayl pentakis (3-hydroxybutanoate) (10 g, 50 mmol, 1 eq.) was introduced into a stirred tank reactor and ethyl acetate (10.6 g, 3 eq.) was added. DABCO (7.1 mg, 0.1 mmol, 0.0013 eq.) was added to the suspension. Subsequently, diketene (8.1 g, 100 mmol, 2 eq.) was slowly dosed to the reaction mixture at 50° C. The dosing rate was adjusted in order to maintain an internal temperature of 48-52° C. After complete addition the mixture was maintained at an internal temperature of 50° C. for an additional 12 h and the solvent was evaporated. Finally the reaction mixture was cooled to room temperature and analyzed. A mixture of the diesterified (2R,3R,4S)-pentane-1,2,3,4,5-pentayl pentakis (3-hydroxybutanoate) was isolated as a yellow-orange oil (16.4 g, 44%). 1H NMR (400 MHz, DMSO-d₆) δ ppm 1.22 (m, 15H), 2.16 (m, 23H), 2.67 (m, 8H), 3.53 (m, 6H), 3.68 (m, 7H), 4.11 (m, 1H), 4.21 (m, 3H), 5.15 (m, 4H), 5.29 (m, 2H), 5.43 (m, 2H).

### Example 29

(2R,3R,4S)-pentane-1,2,3,4,5-pentayl pentakis (3-hydroxybutanoate) (10 g, 50 mmol, 1 eq.) was introduced into a stirred tank reactor and ethyl acetate (10.6 g, 3 eq.) was added. DABCO (7.1 mg, 0.1 mmol, 0.0013 eq.) was added to the suspension. Subsequently, diketene (12.1 g, 150 mmol, 3 eq.) was slowly dosed to the reaction mixture at 50° C. The dosing rate was adjusted in order to maintain an internal temperature of 48-52° C. After complete addition the mixture was maintained at an internal temperature of 50° C. for an additional 12 h and the solvent was evaporated. Finally the reaction mixture was cooled to room temperature and analyzed. A mixture of the triesterified (2R,3R,4S)-pentane-1,2,3,4,5-pentayl pentakis (3-hydroxybutanoate) was isolated as a yellow-orange oil (22.1 g, 53%). 1H NMR (400 MHz, DMSO-d₆) δ ppm 1.21 (m, 15H), 2.17 (m, 14H), 2.65 (m, 6H), 3.54 (m, 4H), 3.68 (m, 4H), 4.10 (m, 1H), 4.22 (m, 2H), 5.14 (m, 3H), 5.29 (m, 1H), 5.44 (m, 1H).

### Example 30

(2R,3R,4S)-pentane-1,2,3,4,5-pentayl pentakis (3-hydroxybutanoate) (10 g, 50 mmol, 1 eq.) was introduced into a stirred tank reactor and ethyl acetate (10.6 g, 3 eq.) was added. DABCO (7.1 mg, 0.1 mmol, 0.0013 eq.) was added to the suspension. Subsequently, diketene (16.2 g, 200 mmol, 4 eq.) was slowly dosed to the reaction mixture at 50° C. The dosing rate was adjusted in order to maintain an internal temperature of 48-52° C. After complete addition the mixture was maintained at an internal temperature of 50° C. for an additional 12 h and the solvent was evaporated. Finally the reaction mixture was cooled to room temperature and analyzed. A mixture of the tetraesterified (2R,3R,4S)-pentane-1,2,3,4,5-pentayl pentakis (3-hydroxybutanoate) was isolated as an orange solid suspended in an orange oil (23.5 g, 51%). 1H NMR (400 MHz, DMSO-d₆) δ ppm 1.22 (m, 15H), 2.17 (m, 19H), 2.66 (m, 8H), 3.54 (m, 6H), 3.69 (m, 5H), 4.11 (m, 1H), 4.22 (m, 3H), 5.13 (m, 4H), 5.29 (m, 2H), 5.42 (m, 2H).

1. A compound of formula 1 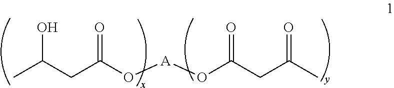
wherein A is derived from an organic polyol with at least 3 hydroxyl groups, x is at least 1, y is at least 1, and x+y is from 3 to the number of hydroxyl groups of the initial organic polyol A,
wherein the ratio of x to y is not 1:1 (x+y), and
wherein the organic polyol is selected from a linear or branched C2-12 alkyl substituted with at least 3 hydroxyl groups or a C3-8 cycloalkyl substituted with at least 3 hydroxyl groups.

2. A process for the preparation of the compound of formula 1 of claim 1, 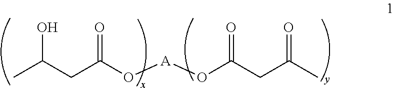
wherein A is derived from an organic polyol with at least 3 hydroxyl groups, x is at least 1, y is at least 1, and x+y is from 3 to the number of hydroxyl groups of the initial organic polyol A,
wherein the ratio of x to y is not 1:1 (x≠y), and
wherein the organic polyol is selected from a linear or branched C2-12 alkyl substituted with at least 3 hydroxyl groups or a C3-8 cycloalkyl substituted with at least 3 hydroxyl groups;
wherein the process comprises:
(i) reacting an organic polyol of formula 2 with diketene 3 resulting in the formation of a compound according to formula 4; 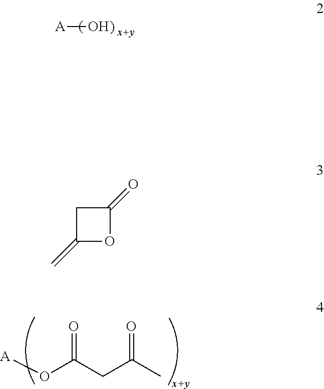
and
(ii) partially hydrogenating the compound of formula 4 by reacting the compound of formula 4 with hydrogen in the presence of a catalyst resulting in the formation of the compound according to formula 1.

3. A process for the preparation of the compound of formula 1 of claim 1, 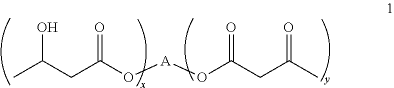
wherein A is derived from an organic polyol with at least 3 hydroxyl groups, x is at least 1, y is at least 1, and x+y is from 3 to the number of hydroxyl groups of the initial organic polyol A,
wherein the ratio of x to y is not 1:1 (x≠y), and
wherein the organic polyol is selected from a linear or branched C2-12 alkyl substituted with at least 3 hydroxyl groups or a C3-8 cycloalkyl substituted with at least 3 hydroxyl groups;
wherein the process comprises:
(i) partially reacting an organic polyol of formula 2 with diketene 3 resulting in the formation of a compound according to formula 5; 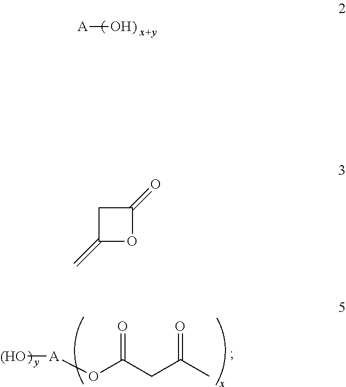
(ii) reacting the compound of formula 5 with hydrogen in the presence of a catalyst resulting in the formation of the compound according to formula 6 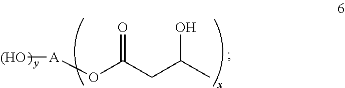
and
(iii) partially reacting the compound of formula 6 with diketene 3 resulting in the formation of the compound according to formula 1.

4. A compound of formula 7 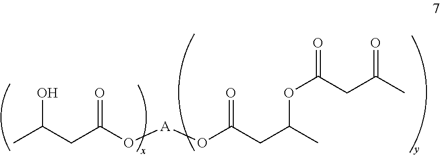
wherein A is derived from an organic polyol with at least 3 hydroxyl groups, x is at least 1, y is at least 1, and x+y is from 3 to the number of hydroxyl groups of the initial organic polyol A, and
wherein the organic polyol is selected from a linear or branched C2-12 alkyl substituted with at least 3 hydroxyl groups or a C3-8 cycloalkyl substituted with at least 3 hydroxyl groups.

5. A process for the preparation of the compound of formula 7 of claim 4, 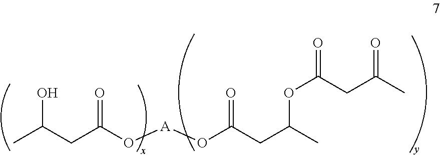
wherein A is derived from an organic polyol with at least 3 hydroxyl groups, x is at least 1, y is at least 1, and x+y is from 3 to the number of hydroxyl groups of the initial organic polyol A, and
wherein the organic polyol is selected from a linear or branched C2-12 alkyl substituted with at least 3 hydroxyl groups or a C3-8 cycloalkyl substituted with at least 3 hydroxyl groups;
wherein the process comprises:
(i) reacting an organic polyol of formula 2 with diketene 3 resulting in the formation of a compound according to formula 4; 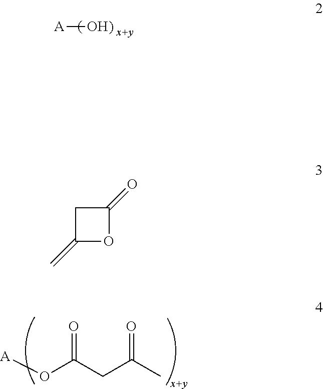
(ii) reacting the compound of formula 4 with hydrogen in the presence of a catalyst resulting in the formation of a compound according to formula 8; 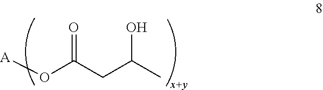
(iii) partially reacting the compound according to formula 8 with diketene 3 resulting in the formation of a compound according to formula 7.

6. A compound of formula 10 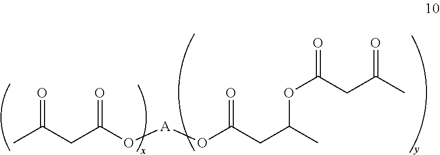
wherein A is derived from an organic polyol with at least 3 hydroxyl groups, x is at least 1, y is at least 1, and x+y is from 3 to the number of hydroxyl groups of the initial organic polyol A, and
wherein the organic polyol is selected from a linear or branched C2-12 alkyl substituted with at least 3 hydroxyl groups or a C3-8 cycloalkyl substituted with at least 3 hydroxyl groups.

7. A process for the preparation of the compound of formula 10 of claim 6, 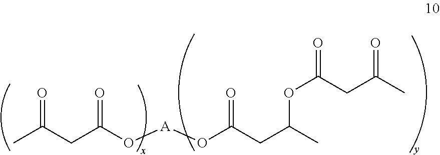
wherein A is derived from an organic polyol with at least 3 hydroxyl groups, x is at least 1, y is at least 1, and x+y is from 3 to the number of hydroxyl groups of the initial organic polyol A, and
wherein the organic polyol is selected from a linear or branched C2-12 alkyl substituted with at least 3 hydroxyl groups or a C3-8 cycloalkyl substituted with at least 3 hydroxyl groups;
wherein the process comprises:
(i) partially reacting an organic polyol of formula 2 with diketene 3 resulting in the formation of a compound according to formula 11; 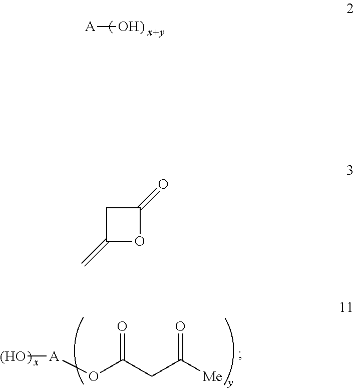
(ii) reacting the compound of formula 11 with hydrogen in the presence of a catalyst resulting in the formation of the compound according to formula 12 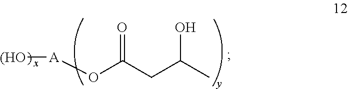
and
(iii) reacting the compound of formula 12 with diketene 3 resulting in the formation of the compound according to formula 10.

8. The compound according to claim 1, wherein x+y is equal to the number of hydroxyl groups of the initial polyol A.

9. A compound of formula 9 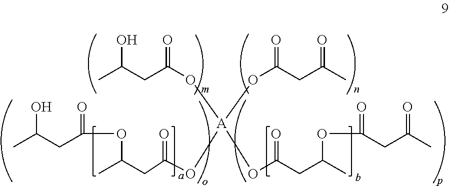
wherein
A is derived from an organic polyol with at least 3 hydroxyl groups,
m is 0, 1, 2, 3, 4, or 5,
n is 0, 1, 2, 3, 4, or 5,
o is 0, 1, 2, 3, 4, or 5,
p is 0, 1, 2, 3, 4, or 5,
at least two of m, n, o, and p are not 0,
m+n+o+p is from 2 to the number of hydroxyl groups of the initial organic polyol A,
a is 1-10,
b is 1-10,
$$n + p \neq a + b + m + o ,$$
and
wherein the organic polyol is selected from a linear or branched C2-12 alkyl substituted with at least 3 hydroxyl groups or a C3-8 cycloalkyl substituted with at least 3 hydroxyl groups.

10. (canceled)

11. The compound according to claim 1, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

12. The compound according to claim 10, wherein the C₃₋₈ cycloalkyl substituted with at least 3 hydroxyl groups is selected from the group consisting of cyclopentanetriol, cyclohexanetriol, cyclopentanetetrol, cyclohexanetetrol.

13. (canceled)

14. (canceled)

15. (canceled)

16. (canceled)

17. The compound according to claim 1, wherein the organic polyol is selected from the group consisting of glycerol, sorbitol, xylitol, mannitol, erythritol, maltitol, glucose, glucitol, ribulose, pentaerythritol, trimethylolpropane.

18. The compound according to claim 1, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.

19. The compound according to claim 9, wherein at least one of:
m is 0, 1, 2, or 3;
n is 0, 1, 2, or 3;
o is 0, 1, 2, or 3; or
p is 0, 1, 2, or 3.

20. The compound according to claim 9, wherein at least three of m, n, o, and p are not 0.

21. The compound according to claim 9, wherein: m+n+o+p is equal to the number of hydroxyl groups of the initial polyol A; or m+n+o+p is less than the number of hydroxyl groups of the initial polyol A.

22. The compound according to claim 9, wherein at least one of: a is 1; or b is 1.

23. The compound according to claim 1, wherein all b-hydroxyl butyric acid ester units are in R-configuration.

24. The compound according to claim 4, wherein x+y is equal to the number of hydroxyl groups of the initial polyol A.

25. The compound according to claim 6, wherein x+y is equal to the number of hydroxyl groups of the initial polyol A.

26. The compound according to claim 4, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

27. The compound according to claim 26, wherein the C₃₋₈ cycloalkyl substituted with at least 3 hydroxyl groups is selected from the group consisting of cyclopentanetriol, cyclohexanetriol, cyclopentanetetrol, cyclohexanetetrol.

28. The compound according to claim 4, wherein the organic polyol is selected from the group consisting of glycerol, sorbitol, xylitol, mannitol, erythritol, maltitol, glucose, glucitol, ribulose, pentaerythritol, trimethylolpropane.

29. The compound according to claim 4, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.

30. The compound according to claim 4, wherein all b-hydroxyl butyric acid ester units are in R-configuration.

31. The compound according to claim 6, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

32. The compound according to claim 31, wherein the C₃₋₈ cycloalkyl substituted with at least 3 hydroxyl groups is selected from the group consisting of cyclopentanetriol, cyclohexanetriol, cyclopentanetetrol, cyclohexanetetrol.

33. The compound according to claim 6, wherein the organic polyol is selected from the group consisting of glycerol, sorbitol, xylitol, mannitol, erythritol, maltitol, glucose, glucitol, ribulose, pentaerythritol, trimethylolpropane.

34. The compound according to claim 6, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.

35. The compound according to claim 6, wherein all b-hydroxyl butyric acid ester units are in R-configuration.

36. The compound according to claim 9, wherein the linear or branched C₂₋₁₂ alkyl substituted with at least 3 hydroxyl groups is selected from the group consisting of glycerol, trimethylolpropane, butanetriol, 2-methyl-propanetriol, pentanetriol, 3-methyl-pentanetriol, hexanetriol, pentaerythritol, butanetetrol, pentanetetrol, hexanetetrol, hexanepentol.

37. The compound according to claim 36, wherein the C₃₋₈ cycloalkyl substituted with at least 3 hydroxyl groups is selected from the group consisting of cyclopentanetriol, cyclohexanetriol, cyclopentanetetrol, cyclohexanetetrol.

38. The compound according to claim 9, wherein the organic polyol is selected from the group consisting of glycerol, sorbitol, xylitol, mannitol, erythritol, maltitol, glucose, glucitol, ribulose, pentaerythritol, trimethylolpropane.

39. The compound according to claim 9, wherein the organic polyol has from 3 to 10 hydroxyl groups, preferably from 3 to 8 hydroxyl groups such as from 3 to 7 hydroxyl groups, from 3 to 6 hydroxyl groups, more preferably from 3 to 5 hydroxyl groups, or from 3 to 4 hydroxyl groups.

40. The compound according to claim 9, wherein all b-hydroxyl butyric acid ester units are in R-configuration.
